(19) United States
(12) Patent Application Publication
(10) Pub. No.: US 2002/0182600 A1
(43) Pub. Date: Dec. 5, 2002

(54) Method for assaying biological and other constituents using synthetic nucleounits in lateral flow, liquid, and dry chemistry techniques

(76) Inventor: Jack V. Smith, Arden, NC (US)

Correspondence Address: JACK V. SMITH, P.O. BOX 156, Arden, NC, 28704, US

(21) Appl. No.: 09/829,563

(22) Filed: Apr. 11, 2001

(51) Int. Cl. C12Q 1/68

(52) U.S. Cl. 435/6

(57) ABSTRACT

The present invention is a method for the use of particles made up of nucleotides or fragments of base groups of DNA and RNA molecules herein referred to as synthetic nucleounits which can be used as recognition molecules with specificity and sensitivity significantly greater than that of antibodies which are used in clinical diagnostics, biotechnology, and research.

### BACKGROUND OF THE INVENTION

1. Field of the Invention

The present invention is a method for the use of particles made up of nucleotides or fragments of base groups of DNA and RNA molecules herein referred to as synthetic nucleounits which can be used as recognition molecules with specificity and sensitivity significantly greater than that of antibodies which are used in clinical diagnostics, biotechnology, research and other uses.

Antibodies are a class of molecules that provide molecular recognition in a wide range of applications and have been known in the art and predates the 1950's. By definition antibodies are any substance which is formed in the body and liberated in the blood and is antagonistic to the presence of antigens.

The present arts novel method for the manufacture and development of synthetic nucleounits can replace the usage of all antibodies and enzymes used in molecular recognition devices such as clinical diagnostic assays, drugs of abuse assays, research and development, diagnosis and treatment of disease to name a few. These devices will provide a more sensitive, precise, stable and cost effective means for rapid analysis in all areas of clinical diagnostics and biotechnology. The present art could save the biotechnology industry billions of dollars in labor, manufacturing, and other cost savings not to mention the added benefit of a more stable and sensitive reagent and diagnostic systems. The nucleounit technology can be used to detect specific analytes of interest. That is to say for example that a particular synthetic nucleounits designed to be used for the detection of glucose in urine or use to detect HIV in blood, proteins, viruses, drugs of abuse and drug metabolites, therapeutic drugs, clinical chemistry targets, toxins or poisons, immunochemistry and radioimmunochemistry targets to name a few. Billions of dollars are spent every year on the manufacture and development of antibodies using animals raised on farms. For example, when animals (sheep, rabbits, cows, etc.) are subjected to the antigen of interest by a biotech firm the animal will develop antibodies to the antigen. Then the animal will be bled on a routine basis and its blood will then be separated and the antibodies removed. Animal rights groups should be in support of the present art because it will do away with the need and use of animals and animal farms for the production and manufacture of antibodies. The technology can also do away with the need to use radioactive isotopes that are currently used in radioimmunosassays. Subsequently producing a lot less hazardous waste in our landfills.

2. Description of the Related Art

The present art provides a method for the replacement of antibodies for clinical diagnostics, biotechnology, drugs of abuse testing, and treatment millions of individuals using antibodies.

A thorough search of patents and research revealed no relative art (i.e., prior art) showing any correlation to this technology. The search has included a search of the USPTO (United States Patent Office) data base with no patents issued for the use of particles made up of nucleotides or fragments of base groups of DNA and RNA molecules herein referred to as synthetic nucleounits which can be used as recognition molecules with specificity and sensitivity significantly greater than that of antibodies which are used in clinical diagnostics, biotechnology, research and other uses. However, the following art will be mentioned to further illustrate the novelty of the present art and the obvious advancement to the current art. The following patents, without exception do not mention the use of urine as the test matrix for detecting specific analytes of interest using nucleounits.

It is known in the art that the urine matrix is very complex and consists of many urinary constituents which create strong buffering and interference problems (e.g. cannibal-like enzymes such as protease) that have to be overcome to provide a method that can be used for the general population with precision and accuracy. Simply because a technique can accommodate a liquid sample does not imply that it can be successfully used with any liquid test matrix. Such successful adaptation of test techniques to accurately deal with specific sample matrices aren't often “obvious” to any scientist. The same can be said of certain nucleounits, research as shown us that nucleases may affect the ability of the nucleounits to measure analytes of interest in biological fluids. However, the present art will demonstrate in detail the formulations and techniques developed that will overcome these type of interferences.

U.S. Pat. No. 4,575,486, claims the detection of red and white blood cells coated with Tamm-Horsfall Protein (THP) for the purposes of trying to determine the origination of red and white blood cells. This patent claims to be able to do this by the use of an antibody to form antibody-antigen complexes that are detected by visual, fluorescent or radioactive techniques in a liquid reagent. This patent is fatally flawed and is of little if any clinical value because it is well known in the art that normal urine is supposed to be free of RBC's, WBC's and other cellular constituents and only a minute amount of THP coated RBC'S and WBC's ever enter the urinary tract. On the other hand, the vast majority of the RBC's and WBC's found in urine are due to infections of the bladder or urethra; these cells are not THP-coated thereby making the detection of THP-coated red and white blood cells improbable if not impossible by this method. This patent does not disclose a method for the determination of RBC's and WBC's in urine or any other matrix using nucleounits. This patent also fails to teach the use of ultra-violet or visible (i.e. UV-Vis) colorimetric, spectrophotometric, and reflectance techniques for the determination of RBC's and WBC's in urine utilizing dry chemistry dipsticks or lateral flow devices (LFD) for manual determination or aqueous reagent formulas compatible with an automated chemistry analyzer in conjunction with nucleounits sensitive to the presence of RBC's and WBC's.

Another patent, U.S. Pat. No. 3,961,039, is a stain for urinary sediments utilized on a glass slide. It does not teach a method of determining the presence of any analyte of interest using nucleounits. It does not teach dipstick or LFD assay techniques. It also does not describe a liquid reagent that is compatible with automated chemistry analyzers. In fact, attempting to adapt its formulations to an automated analyzer would cause significant damage to the optics and test cuvettes. This patent fails to disclose the use of fluorescent, radioactive, UV-Vis, colorimetric, spectrophotometric, and reflectance techniques for the determination of analyte that is used in a clinical diagnostic assay, treatment, or research in conjunction with a nucleounit. This disclosure also fails to mention any modes of determination of any analyte of interest using nucleounits by any measurable means including enzymatic, antibody-antigen, calorimetric, or other means.

Another patent, U.S. Pat. No. 4,446,232, is an enzyme immunoassay technique using detection zones for the determination of the presence of antibodies and does not teach or suggest the method of determining the presence of any analyte of interest using nucleounits. It also fails to teach assay techniques using a dry chemistry dipstick or LFD, or a liquid reagent compatible with automated chemistry analyzers. This patent also fails to mention any use of ultra-violet, calorimetric, spectrophotometric and reflectance techniques for the determination of any analyte of interest using nucleounits in urine or any other matrix (i.e., blood, etc.). This disclosure also fails to mention any modes of determination of urinary analytes by any measurable detectable means such as enzymatic, antibody-antigen, colorimetric, or other chemical means.

Another U.S. Pat. No. 4,786,589, is an immunoassay using formazan-prelabled reactants and does not teach or suggest the method of determining the presence of urine or other biological fluid analytes by the use of nucleounits in conjunction with dry chemistry dipstick (DCD) or LFD, or a liquid reagent compatible with automated analyzers. This art requires the sample to be premixed with a labeled primary protein having a specific affinity for the analyte of interest. This patent does describe dipsticks. The pretreatment step, however, creates significant problems. This step makes application to an automated chemistry analyzer fruitless, because of the additional labor it requires. This step also makes it unacceptable for point-of-care and home use with DCD's and LFD's, because of the potential for errors. Again, this method fails to teach a method for the determination of analytes of interest using nucleounits. This patent also fails to teach the use of UV-Vis calorimetric, spectrophotometric, and reflectance techniques for any determinations in urine or any other matrix using microunits. It also doesn't describe the use of DCD's or LFD's for manual determination, or aqueous reagents for use on automated chemistry analyzers. This disclosure also fails to teach a method for determination of urinary or other biological matrices analytes via enzymatic, antibody-antigen, calorimetric, or other chemical means in conjunction with the use of nucleounits.

Another patent, U.S. Pat. No. 3,603,957, teaches the use of assay test strips but page again fails to disclose a method for the determination of urinary or other biological matrices analytes. This patent also fails to teach the use of UV-Vis, calorimetric, spectrophotometric and reflectance techniques for the determination of urinary or other biological matrices analytes using nucleounits. It also doesn't teach the use of DCD's or LFD's or aqueous formula for use on an automated instrument. This disclosure also fails to teach a method for determination of urinary or other biological matrices analytes via enzymatic, antibody-antigen, colorimetric, or other means in conjunction with the use of nucleounits.

In the literature and prior art, techniques such as ELISA and other methods have been used to detect certain markers in blood, however, these methods have no relevant bearing on the present device. ELISA is a technique that coats a micro-titer well plate with antibody for the particular analyte of interest. This immobilized method has no similarity or relevance to the DCD, LFD, or aqueous reagent using nucleounits for automated instruments. It would be impossible to grind up a micro-titer well plate and somehow liquefy it for use on an automated instrument for the quantitative determination of urinary analytes of interest using nucleounits. It is also improbable to apply this logic to a dipstick or LFD assay technique. The afore mentioned techniques, along with two-site immunochemiluminometric techniques, have no bearing on the present device for obvious reasons. For instance, the ELISA is an immobilized method, and the reaction mixture cannot, therefore, be moved from one area to another (like a carrier-free aqueous reagent which is transferred from a reagent container to a reaction cuvette of an automated chemistry analyzer).

The two-site immunochemiluminometric techniques can assay for target proteins by pretreating each sample with a specific binding protein; the bound and free fractions must then be separated by another antibody-antigen reaction, and then linked to magnetic particles and measured by some means. This process offers numerous obstacles. First, this technique is very time consuming, and not applicable to current chemistry automation or manual one-step DCD's and LFD's using nucleounits. Obviously, the present device represents a significant advancement over these older techniques including ELISA, microscopic analysis, electrophoresis, two-site immunochemiluminometrics, immunofluorescent staining, zone detection, slide staining, and multiple detection layers.

Again, compare ELISA versus an aqueous reagent applied to an automated chemistry analyzer for quantitative analysis. The former takes up to two hours or more. The latter technology using the nucleounits specific for a particular target can yields results in a few seconds to minutes along with quality controls to validate the accuracy of the data.

It is well known in the art, that these two methodologies are very time consuming and labor intensive, and require hours to complete analysis on a single sample; they also require complex, and expensive instrumentation. As a whole ELISA and HPLC are not effective assays for high volume testing, small clinics or doctors' offices, or home testing because of costs, sophisticated equipment and associated skill required, and analysis time. In particular, HPLC, is very complex and requires many assay steps including sample clean up, derivatization, purification, and which result in low variable yields. ELISA requires even more steps prior analysis, including preparation of a micro titer plate, predilution, and numerous serial dilution's, PBS (phosphate buffered saline) pre-incubation, incubation with a secondary antibody, addition of a color reagent, interruption of the color reaction, and finally the absorbance is determined. Obviously, these multi-step assays are very tedious and time consuming, and require significant analytical skills.

As the foregoing illustrates there is a need in the art for rapid analysis using the nucleounits targeted to a specific analyte of interest in urine and other matrices. In general assays should be simple and inexpensive to perform in order to make them widely available. Assay techniques that fill this description include dry chemistry dipsticks (DCD), lateral flow devices (LFD), and aqueous liquid reagents compatible with automated chemistry analyzers (ACA). The present device provides an inexpensive, readily available, rapid analysis for specific analytes of interest. This technology will save millions in cost of reagents, stability, accuracy, and sensitivity that this new and novel technology will provide. The waste of animals and horrors of animal testing and their usage for the development of antibodies can end. When animals are used or cell lines for the development of antibodies toxins and non-specific molecules are produced which cause interference in diagnostics assay. The interfering molecules that are produced from the animal and cell lines will interfere with the sensitivity and high affinity goals of an analyte specific target to be used in diagnostics assay or for other uses. The nucleounits do not depend on animal, cell, or in vivo conditions.

The synthetic nucleounit technology of the present art therefore, can prevent the injury and loss of life due to this disease, can be used to design novel and specific assay for the detection of analytes of interest without the use of animals or animal cell lines. The present art use of DCD or LFD techniques provides a method for the general consumer (patient) to save money and still receive the health care needed by providing a test result for dollars at home or in the clinic versus the current art which costs hundreds of dollars. Ultimately this could save the consumer, nation and world economy millions of dollars. The clinical treatment and diagnostics of the detection of diseases such as AIDS, CMV, and Hepatitis are very expensive and time consuming as is well known in the art. Early detection of these diseases, and optimization of treatment is imperative to save dollars and lives.

### SUMMARY OF THE INVENTION

The present invention is a method for the use of particles made up of nucleotides or fragments of base groups of DNA and RNA molecules herein referred to as synthetic nucleounits (nucleounits) which can be used as recognition molecules with specificity and sensitivity significantly greater than that of antibodies which are used in clinical diagnostics, biotechnology, research and other uses.

The present arts novel method for the manufacture and development of synthetic nucleounits can replace the usage of all antibodies and enzymes used in molecular recognition devices such as clinical diagnostic assays, drugs of abuse assays, research and development, diagnosis and treatment of disease to name a few. These devices will provide a more sensitive, precise, stable and cost effective means for rapid analysis in all areas of clinical diagnostics and biotechnology. The present art could save the biotechnology industry billions of dollars in labor, manufacturing, and other cost savings not to mention the added benefit of a more stable and sensitive reagent and diagnostic systems. The nucleounit technology can be used to detect specific analytes of interest. That is to say for example that a particular synthetic nucleounits designed to be used for the detection of glucose in urine or use to detect virulent disease causing viruses such as HIV, proteins, viruses, drugs of abuse, drug metabolites, and therapeutic drugs such as the following, but not limited to the following; cocaine, opiates, gamma-hydroxybutyric acid, cannabinoids, benzodiazepines, acetaminophen, amikacin, aminocaproic acid, amitriptyline, amobarbital, amphetamine, bromide, caffeine, carbamazepine, carbenicillin, chloral hydrate, chloramphenicol, chlordiazepoxide, chlorpromazine, cimetidine, clonazepam, clonidine, clorazepate, cocaine, cocaine metabolites, ethanol, methanol, or other forms of alcohol, codeine, cyclosporine, desipramine, dexamethsone, diazepam, digoxin, diphenylhydantoin, disopyramide, doxepin, ephedrine, ethchlorvynol, ethosuximide, fenoprofen, flecainide, flurazepam, gentamicin, glutethimide, hydromorphone, ibuprofen, imipramine, isoniazid, kanamycin, lidocaine, lithium, lorazepam, lysergic acid, meperidine, meprobamate, methadone, methamphetamine, methaqualone, methotrexate, methsuximide, methyldopa, methyprylon, morphine, n-acetylprocainamide, netilmicin, nortriptyline, oxazepam, oxycodone, paraldehyde, paraquat, pentazocine, pentobarbital, phenacetin, phencyclidine, Phenobarbital, phensuximide, phenylbutazone, phenylpropanolamine, phenytoin, primidone, procainamide, propoxyphene, propranolol, protripyline, quinidine, salicylates, secobarbital, theophylline, thiocynate, thiopental, thioridazine, tobramycin, tolbutamide, valproic acid, vancomycin, or warfarin, . . . etc., or clinical chemistry targets such as the following, but not limited to the following; cholesterol, triglycerides, glucose, adrenocortocotropic hormone, alanine, alanine aminotransferase, albumin, aldolase, aldosterone, amylase, amyloid-associated protein, androstenedione, angiotnesis, antidiuretic hormone, antithrombin, antitrypsin, apolipoprotein, ascorbic acid, bile acids, bilirubin, c-peptide, calcitonin, calcium, cancer antigen 125, carboxyhemoglobin, carotene, catecholamines, cholic acid, cholyglycine, chromium, chymotrypsin, complement components, coproporhyrin, corticobinding globulin, corticosterone, cortisol, c-peptide, c-reactive protein, creatine, creatinine, creatine kinase, cyclic AMP, cystine, cysteine, dehydroepiandrosterone, dehydroepiandrosterone sulfate, deoxycholic acid, 11-deoxycortocosterone, 11-deoxycortisol, dihydrotestosterone, estradiol, estriol, estrogen, estrone, fecal fat, fatty acids, ferritin, fetoprotein, fibrinogen, folate, follicle stimulating hormone, thyroxine, triiodothyronine, fructose, fructosamine, galactose, gastric acid, gastrin, glucagons, glucose-6-phosphate, glutamine, glutamyltransferase (GGT), glutathione, hemoglobin, glycerol, glycine, glycolic acid, gold, growth hormone, haptoglobin, high-density lipoproteins, hemopexin, hemocystein, homocysteine, homogentisic acid, homovanillic acid, hydrogen sulfide, 17-hydroxycortocosteriods, 5-hydroxyindoleacetic acid, 17-hydroxyprogesterone, hydroxyproline, immunoglobins, insulin, iron, isocitrate dehydrogenase, isoleucine, 17-ketogenic steroids, ketone bodies, lactate, lactate dehydrogenase, lactose, LDL-cholesterol, lecithin, leucine, leukocyte, lipase, lipoproteins, lutropin, lysozyme, macroamylase, magnesium, melanin, metanephrine, methionine, metyrapone, microsomal antibodies, antibodies, molybdenum, mucoploysaccharide, myelin basic protein, myoglobin, methemoglobin, niacin, nickel, nitrite, nitrogen, nonprotein nitrogen, normetanephrine, blood, orosomucoid, oxalate, oxytocin, pancreatice polypeptide, pantothenic acid, parathyroid hormone, pentachloropehnol, pentoses, pepsinogen, phenols, phenolsulfonphthalein, phenylalanine, acid phosphatase, alkaline phosphatase, phosphofructokinase, phospholipids, placental lactogen, plasminogen, porphobilinogen, prealbumin, pregnanediol, pregnanetriol, pregnenolone, progesterone, porinsulin, properdin, prostaglandins, prostate-specific antigen, portoporphyrin, pseudocholinesterase, pyruvic acid, renin, reverse triiodothyromine, rheumatoid factor, riboflavin, secretin, selenium, serotonin, somatomedin c, sucrose, testosterone, tetrahydrocortisol, tetrahydrodeoxycortisol, thallium, thyroglobin, thyroid antibodies, thyroxine binding globulin, thyroxine, transcortin, transferring, transketolase, transthyretin, thyrotropin-releasing hormone, triglycerides, triiodothryonine, tyrosine, urea, urea nitrogen, uric acid, uricase, urobilinogen, uroporphyrin, valine, vanillymandelic acid, vasoactive intestinal polypetide, human chorionic gonadotropin, mass creatinine kinase, vitamins, xylose, or zinc, toxins or poisons such as the following, but not limited to the following; cyanide, formaldehyde, ethylene glycol, lead, mercury, xylene, and immunochemistry targets and infectious disease targets such as the following but not limited to the following: human immunodeficiency virus (HIV), cytomegalovirus (CMV) IgG, cytomegalovirus (CMV) IgM, herpes simplex virus (types 1 and 2) IgG, rubella IgG, rubella, IgM, toxoplasma IgG, toxoplasma IgM, amebiasis, Epstein-barr early antigen, Epstein-barr EBNA IgG, Epstein-barr VCA IgG, Epstein-barr VCA IgM, helicobacter pylori-IgG, legionella IgG/IgM/IgA, mycoplasma IgG, mycoplasma IgM, varicella zoster virus (VZV), or autoimmune diseases such as the following but not limited to the following; antinuclear antibodies (ANA), antineutrophil cytoplasmic antibodies (ANCA), anti-cardiolipin, anti-dsDNA, anti-Jo-1, anti-Scl-70, anti-Sm (Smith antigen), anti-Sm/RNP, anti-SS-A/Ro, anti-SS-B/La, extractable nuclear antigen (ENA), myeloperoxidase IgG, proteinase-3 IgG, and Rheumatoid Factor. The measurable test means of the present device produces a visual, spectrophotometric, turbidimetric, fluorescence, or reflectance result by the combination of the nucleounits with its target resulting in the production of a measurable response.

Rapid, one-step home and physician's office testing currently takes the form of DCD's and LFD's. These devices consist of absorbent carriers, usually paper, which has been impregnated with all of the chemicals needed for the detection reaction. After dipping the DCD into a body fluid, or adding a drop of fluid to the test pad, a color reaction takes place. Because of the importance of achieving rapid results dipsticks have been developed to detect various disease markers in body fluids. Another rapid test device, the LFD, is very similar to a dipstick in principle. This device combines the DCD with some aspects of thin layer chromatography (TLC) principles. After dipping one end of the LFD into a sample, the urine migrates up the paper (or absorbent material) to the reactive sites containing reagents (reactive ingredients). The urine constituents react with the assay reagents during the migration process and yield visible results. Automated liquid chemistry analysis utilizes aqueous reagent mixtures used in conjunction with automated chemistry analyzers. This assay system utilizes microliter amounts of reagents and samples and produces accurate results on hundreds or thousands of samples per hour with minimal labor (e.g. 1 technologist per instrument). For the detection of analytes of interest as discussed above in urine, the sensitivity of the test is of decisive importance and, furthermore it is also desirable. The dipstick test or LFD using the present arts synthetic nucleounit technology has a qualitative to quantitative sensitivity range of or less dependent upon the target of choice. On the other hand, the automated liquid test has a sensitivity range of much greater than that of the dipstick or LFD technologies. The sensitivity of the synthetic nucleounits to their counterparts the antibodies for a specific target or analyte of interest is much greater. The nucleounits can discriminate molecules or their targets of interest that are closely related to cognate targets at the atomic level.

Examination of patents and published research reveal no relative art (i.e., prior art) even slightly resembling this technology or the use of this technology as described in the present specification. No chemical test means has been described prior to this disclosure which can perform the tasks this new art can.

Briefly stated, the present invention relates to test devices for measurement of analytes of interest using nucleounits in fluids or others such as biological fluids to include but not limited to urine, blood, saliva, or other fluids that come from the human body or other animals, and the procedures for making said test means. This invention is in the fields of clinical diagnostics and treatment. More specifically, this invention provides dry chemistry dipsticks (DCDs or on-site test modules), thin layer chromatographic dry chemistry technology (LFDs), and aqueous, liquid chemistry reagents that quantitate analytes of interest. For instance the analyte of interest could be HIV and the present art is designed to determine if the test subject has been exposed to the HIV virus that causes AIDS on biological samples (e.g. urine, serum, and blood). This new art can utilize aqueous, biological specimens including urine, saliva, sweat extracts, blood, and serum. In addition, this invention provides a unique method for analytes of interest or for HIV antibodies, drugs of abuse, clinical chemistry targets, or others for measurement utilizing rapid test devices including the DCD, and LFD thereby enabling in-home testing through over-the-counter (OTC) sales. This is an enormous advancement in the art. These advances and improvements of the present device over the prior art provides the health care testing industry with powerful new clinical and diagnostic tools.

This invention eliminates the need for the costly antibody HPLC, and/or ELISA plate testing and the concomitant long term testing requirement to adequately evaluate treatment efficacy (i.e. 6 to 12 months) currently necessary. This invention also improves the sensitivity, specificity, accuracy, and economics of analysis by applying it principles to DCDs, LFDs, DLFH and aqueous, liquid chemistry reagents. Note, the previous art taken as a whole (e.g., the use of antibodies), does not enable the use of an effective agent to target analytes of interest without the use of animals. As previously explained in detail the use of the present arts nucleounits in assay methods such as dry chemistry dipstick (DCD) format, lateral flow (LFD) format, liquid methodologies provide unforeseen advances in the art of clinical diagnostics such as improvements in sensitivity, specificity, accuracy and stability of the procedures. Antibodies inherent incompatibility with buffers, temperature and other factors of the prior art methods using antibodies also present problems with safety, stability, the use of blood from live animals, the use of animals, and susceptibility to interference from cross reactants to name a few of the inherent problems with the prior art.

The present art yields unexpected results that are achieved by the use of the nucleounit versus antibodies. The results are new (sensitivity with out the use of animals), unexpected and superior in that the results from use of the nucleounit versus antibody provides greater sensitivity and accuracy to the specific target of interest than antibodies.

This new art described herein fills two key needs in two diverse arenas. The first area of need involves physicians' offices and their ability to diagnose disease and illness through the use of dry chemistry assays on biological specimens such as urine, blood or blood products using nucleounits versus antibodies. This dipstick/LFD assay is ideal for this application, because it requires no sophisticated equipment, or training, is much less expensive, and provides immediate results. These test devices can also be utilized by laypersons at home, or in countries in which sophisticated lab work is not possible. The second arena of advantage lies in large, high volume, reference labs. These facilities typically serve as regional test centers, and perform large numbers of tests each day. Such labs would use automated chemistry analyzers in conjunction with aqueous, liquid reagents to reduce technician time, lower cost of testing, and test large numbers of samples in very short time periods. The most important aspect is the present art's ability to assay for analytes of interest without the use of antibodies from animal sources.

The present invention relates to a method that can be used by two different techniques that do not use antibodies but instead use nucleounits. One technique employs dry chemistry technology for DCD'S and LFD'S as outlined above. A second technique employs an aqueous reagent compatible with automated chemistry analyzers currently available to medical labs. As indicated above both of these techniques can be used to measure for analytes of interest allowing the determination of illness or disease. The advantages of the dry chemistry technique include ease of use, semi-quantitative or quantitative results, low cost, and technical improvements (e.g. increased sensitivity, specificity, and accuracy, and reduced interference); no one has SUCCESSFULLY ADAPTED any of the prior art for analyte of interest testing using nucleounits as it applies to dry chemistry applications. This technology is manufactured by impregnating onto absorbent paper the chemical constituents, which have been dissolved in a liquid format, evaporating the liquid, and mounting this “test paper” on a sturdy plastic handle.

The advantages of the liquid reagent technique are true quantitation, reduced cost per test, technical improvements (e.g. increased sensitivity, specificity, and accuracy, and reduced interferences), expanded range of detection (lower and upper limits), and compatibility to chemistry analyzers thereby permitting assay of hundreds of specimens per hour with minimal labor (e.g. one technologist can operate one or two analyzers which are doing twelve or more different assays on up to one thousand samples/hour).

This new art is composed of an indicator(s) (i.e. calorimetric, enzymatic, fluorescent, turbidimetric, radioimmunologic, ion-exchange, or ionic), bounds or conjugated to a nucleounit and buffers. Interference-removing compounds may also be included but are not required for the assay to work effectively. The present art's assay for analytes of interest in urine may be further enhanced by the use of a creatinine, cystatin C, or specific gravity assay performed on the same urine sample. This enhancement permits the use of a random or spot urine instead of collection of a 24-hour sample. The creatinine, cystatin C, or specific gravity value is used to “normalize” or correct the test result for diurnal variations. For example, if the urine were dilute the analyte of interest value would be low and should be adjusted upward to a higher value. And if the urine were concentrated the analyte of interest value would be high and should be lowered. The objective of this procedure is to determine how much of the marker protein is excreted per day. It is known in the art that creatinine and cystatin C as well as other markers are steady state components of human urine and can be used as a reliable source to determine urine concentration. Specific gravity, and osmolality can also provide the same information. Research has revealed no relative prior art to this invention thereby eliminating the obviousness of this novel invention. The current art bears no relation to that which is described herein.

As an example of a target for an “analyte of interest” lets use GHB (gamma-hydroxybutyrate) as the analyte of interest in the following example. Of course the analyte of interest could be changed from GHB, to cocaine metabolite, heparin, creatinine kinase, etc. The first method for measurement of GHB utilizes a nucleounit with specific affinity to GHB conjugated to glucose-6-phosphate dehydrogenase an indicator/substrate sensitive to dehydrogenase activity. The assay is dependent upon the concentration of the GHB and its corresponding effect on the bound dehydrogenase. The use of GHB as the analyte of interest target and glucose-6-phosphate are merely illustrative for this unique invention and other possibilities are possible as has been explained.

This method can include color producing indicator compounds that yield ultra-violet or visible color and can be bound to an GHB nucleounit (nucleounits are nucleotides in chains of 10 or more nucleotides in a specific sequence that bind specific ligands, like antibodies, nucleounits have high affinities and specificities for targets such as GHB or other analytes of interest) that are specific for GHB. These color-producing compounds can be bound to the nucleounit via covalent or ionic bonding to form a GHB nucleounit-indicator complex. Examples of indicators that yield a detectable calorimetric response, and may be used for this purpose include horseradish peroxidase (HRP), tetramethylbenzidine (TMB) para-nitroaniline, glucose-6-phoshpate dehydrogenase (G6PDH), alkaline phosphatase (AP), fluorescein (FITC), tetramethyl rhodamine isothiocynate (TRITC), Biotin, phycoerytherin (PHYCO), and naphthylamine (see examples for additional compounds). When this GHB nucleounit-indicator complex contacts GHB the complex's bond is fractured thereby yielding the colored indicator or the chromogen portion of the complex is activated and will react with other substrate in solution with the complex. In some cases additional reactants may be required to combine with the released compound to produce a colored product. For example, the GHB nucleounit/p-nitroanilide compound would yield the GHB-GHB nucleounit complex and p-nitroaniline; this latter compound will yield a yellow color. In the case of naphthylamine the additional reactants would include sodium nitrite and N-1-naphthylethylenediamine. This secondary reaction would produce a blue color. The nucleounit-indicator complexes are obtained via standard organic synthesis. These assays can be competitive binding assays. The nucleounit-indicator is mixed with sample. This solution will attack any GHB in the sample. Of course the sample could be any liquid or biological fluid. In the case of GHB it has become a popular drug of abuse on college campuses, bars, and dance clubs and is called the “date rape” drug. The abuse of GHB has enormous sociological and economic impact on our society. A typical “date rape” scenario is as follows: The victim(s), usually women, are in a bar, they drink a beverage that has been laced with GHB by a rapist, the victim then becomes catatonic and is usually agreeable with anyone and everyone around them. They can become unconscious and then of course are susceptible to the rapist desires. Later, the victim(s) wakes up completely disoriented, naked and robbed. This type of horrific tragedy is occurring on a daily basis. The victims of “date rape” are not only exposed to the physical assault of rape and robbery, but to the contraction of diseases such as AIDS and STD's. The damage caused and the consequences of such occurrences are immeasurable. Accordingly, a need exists for providing an easy and convenient manner by which to make a determination of the presence of GHB in urine, in a beverage, or other biological fluids or liquids. A further need exists for a convenient manner by which such determinations may be made by using rapid analysis manual techniques (such as a dipstick or lateral flow devices) and automated techniques that will advance the art significantly. And, the most important need is for a device that would detect GHB using just a single assay that does not require an extraction process or lactone conversion. This would be a marked advancement in the art and would result in the savings of millions of dollars to the drug testing laboratories required to perform GC (gas chromatography) or GCMS (gas chromatography mass spectrometry) testing for GHB. With that said the rate at which color is generated over a fixed amount of time is determined, and compared to that produced by standards with known amounts of the GHB, to quantify the concentration of analyte in the unknowns. This quantification can be observed visually, or measured via spectrophotometer.

This method could requires a nucleounit bound to GHB. This assay technique is based on competitive binding between free GHB in the sample and bound GHB conjugated to an enzyme such as glucose-6-phosphate dehydrogenase (G6PDH) contained in part one of the reagent (R-1). The GHB-G6PDH in the R-1 is first mixed with sample. In part two of the reagent system (R-2) the solution contains the substrate, glucose-6-phosphate (G6P), and a co-enzyme, Nicotinamide Adenine Dinucleotide (NAD) and nucleounit specific for GHB. After incubation of the sample with the R-1, part two of the reagent, R-2, is added. If no analyte (GHB) is present in the sample, the nucleotide specific for GHB in the R-2 will bind up all of the GHB-G6PDH in the reagent R-1 and little or no reaction will take place and little or no measurable change in absorbance will take place at 340 nm (nanometers). If GHB is present in the sample, the nucleounit specific for GHB in the R-2 will bind to it leaving the unbound GHB-G6PDH in the R-1 free to interact with the reactants present in the R-2 reagent (e.g., substrate, glucose-6-phosphate (G6P), and a co-enzyme, Nicotinamide Adenine Dinucleotide) and an increasing and significantly measurable reaction will go at a known pace as compared to calibrators and standards containing know concentrations of GHB. This reaction would be measured at 340 nm for the conversion and subsequent reduction of NAD to NADH. Obviously, enzyme reaction kinetics (or enzyme binding to the substrate) reacts proportionately to the amount of GHB present or not in the sample, therefore, its concentration in the sample can be measured in terms of enzyme kinetics; the amount of color generated is proportional to the amount of GHB in the sample. This reagent system of the instant invention (liquid reagent) is intended for use on any automatic chemistry analyzers with open channel capability including Olympus AU 5000 series, Hitachi 700 series, and many others as well as DCDs or LFDs.

An example of the analysis procedure utilizing the reagent system of the instant invention described herein is as follows: the two components of the reagent composition (R-1 and R-2) are placed in the reagent compartment of the analyzer; samples, calibrators, and controls are aliquoted into sample cups which are then placed on the analyzer. An aliquot of 10 uL of each specimen is then pipetted into a single, discrete cuvette followed by the addition of 125 uL of the first reagent, R-1, and mixed; After a specified incubation time of five minutes, 125 uL of the second reagent, R-2, is added to the cuvettes, and mixed. A first spectrophotometer reading is then taken followed by a second after a specified incubation period (i.e. one minute for this example) at the specified wavelength (between 340 and 800 nm). The spectrophotometer readings are then recorded. In this instance the assay is read at 340 nm. The absorbance of samples, and controls are stored and then compared to a standard curve derived from the calibrators' absorbance; this comparison yields quantitative values for the unknowns and controls, which are printed on a report. This method will function for liquid, automated analysis, only. An indicator that yields a visible (measurable) color change is required for dry chemistry dipstick analysis. For example, inclusion of a tetrazolium indicator (e.g. nitro-blue tetrazolium) and an electron carrier (e.g.1-methoxy-5-methylphenazium) will yield a color change in the visible spectrum. This color reaction could be utilized for DCDs and LFDs as well as in the aqueous, liquid reagent system. Another alternative for production of a visual color change would require substitution of G6PDH (conjugated to anti-HIV), its substrate, G6P, and NAD with Galactosidase and 5-bromo-6-chloro-3-indoxyl-beta-D-galactopyranoside will produce a magenta color that increases with increasing concentration of the target marker (e.g. Ntp). Another variation of the above methodology would utilize a fluorescent marker in place of the NAD, and could be measured using fluorescent spectroscopy.

Yet another variation of the present arts assay technique for analysis of analytes of interest in biological fluids utilizing particle-enhanced aggregation (PEA). An example of this technique includes an R-1 which contains nucleounit to the analyte of interest, a nucleounit targeted for cholesterol (n-cholesterol) for example. The R-2 contains microparticles conjugated to cholesterol (p-cholesterol). The reagent's R-1 is mixed with sample. If cholesterol is free in the sample, it binds to the nucleounit (n-cholesterol). The R-2 is then added. The n-cholesterol will not bind to the p-cholesterol because it has been bound to all of the free cholesterol in the sample. The n-cholesterol will not bind to the p-cholesterol, therefore little or no (there could be some free n-cholesterol in solution dependent upon the concentration of the cholesterol in the sample and the concentration of the n-cholesterol in the reagent R-1) formation of particle aggregates will take place between the n-cholesterol and the p-cholesterol. Conversely, the absence of cholesterol increases the absorbance in proportion to the concentration of cholesterol or lack in the sample. Any unbound n-cholesterol is then free to react with the p-cholesterol. This reaction promotes formation of particle aggregates between the p-cholesterol and n-cholesterol. As the aggregation reaction proceeds in the absence of free cholesterol in the sample, the absorbance monitored increases spectrophotometrically. This assay can be monitored spectrophotometrically from 340 to 800 nm. Alternatively, the nucleounits can be chemically bound to the polystyrene microparticles. These cholesterol targeted nucleounit-microparticles will bind to any cholesterol present in the sample, and in this process form aggregates. Therefore, the absorbance of the reaction mixture increases in proportion with the concentration of cholesterol. This absorbance change can be read between 340 and 800 nm. This same unique technology can be used for DCDs and LFDs.

The dry chemistry, on-site assay devices (DCDs) utilizing particle enhanced aggregation for analysis of HIV in biological fluids contain microparticles of uniform size, chemically coupled with antibody to one or more of the markers noted above (e.g. n-cholesterol). In the case of a static dipstick device, the microparticles are also conjugated with an indicator. If the analyte of interest is present, the nucleounit-microparticles bind to it, and simultaneously displace the indicator; this results in the formation of color on the test pad. If no analyte of interest is present, no color forms. Obviously, the amount of color formed is proportional to the amount of analyte of interest present.

When this assay model is adapted to liquid format, the color indicator is not required (but could be used). The microparticles which react with a nucleounit targeted at a specific analyte of interest to form analyte of interest nucleounit-microparticle complexes which will spontaneously combine to form aggregates in the presence of the analyte of interest. The formation of said aggregates will cause an increase in absorbance. Therefore, absorbance (read between 340 and 800 mn) is directly proportional to the analyte of interest concentration which can then be compared to concentration of standards and calibrators that are the same as the particular analyte of interest.

Another type of on-site test methodology utilizing PEA technology combines thin layer chromatography with dry chemistry dipstick technology (i.e. LFDs). In this case, the microparticles are chemically coupled to an nucleounit against a specific analyte (e.g. bile acid) and are colored, but are not conjugated to an indicator. Sample mixes with the microparticles at the base or starting line of the LFD. If bile acids are present, it binds to the nucleounit-microparticle (n-microparticle). This nucleounit-microparticle-bile acid complex (i.e. n-microparticle-ba) then continues wicking up the strip past a result line (1st window) to the validation line (2nd window) which is composed of nucleounit to the nucleounit conjugated to the microparticle (e.g. n-n-microparticle) which is bound to the test strip via a protein. The n-microparticle-ba complex then reacts (binds) to the n-n-microparticle; the end result being a visible colored line formed by the colored microparticles in the second, or validation window. If bile acid is not present in the sample, the n-microparticles wick up the test strip until they reach the result (first) window in which nucleounit targeted to bile acid has been bound to the paper. The n-microparticles then binds to the immobilized nucleounit targeted to bile acid forming a colored line as a result of the colored particles. Please note, however, that nucleounit-microparticles need to exceed the quantity of nucleounit targeted to bile acid bound to the strip in the result window. This excess of n-microparticles, therefore, continue to migrate up the test strip to the validation window where they bind to the n-n-microparticle forming a visible colored line and confirms the test is complete. Note, therefore, that a colored line will form in the validation window in the case of a positive or negative result. On the other hand, no line will form in the result window in the case of a positive result. This technique can be further simplified by eliminating the nucleounit to n-n-microparticle in the validation window. Excess colored microparticles will still congregate at the top of the device thereby forming a visible line, and indicating completion of the test.

Final method(s) for analysis of analytes of interest in fluids. Biological fluids for example could be used. In this example urine is used and an enzyme-labeled nucleounit specific to one or more of the analytes of interest markers. Techniques for conjugation of enzymes to other substances is well known in the art. Many different enzymes or co-enzymes can be utilized for this purpose; for example, galactosidase can be conjugated to a nucleounit specific to thyroxine (T₄). This complex forms the active portion of R-1, and is mixed first with the urine sample. If thyroxine is present in the sample, it binds to the nucleounit, thereby causing inactivation of the enzyme. The R-2 reagent containing a substrate-indicator selected to complement the enzyme (e.g. 5-bromo-6-chloro-3-indoxyl-beta-D-galactopyranoside) is then added to the reaction mixture. The inactivated enzyme will not react with the substrate-indicator and no color will develop. The decrease in color development is directly proportional the concentration of thyroxine present. If no thyroxine is present, the enzyme will remain activated and will interact with the substrate-indicator in the R-2 to produce color (e.g. magenta) which can be measured spectrophotometrically, visibly, or by other means. Again, the amount color produced is proportional to the concentration of thyroxine present in the sample. Please note this method can be adapted to dry chemistry dipstick technology. First, the solid paper matrix is immersed in the R-2 reagent, and finally in the R-1 reagent. Alternatively, two distinct paper matrices, or test pads could be used (one for each immersion solution), and a “sandwich” made by stacking the two pads, one on top of the other. The paper pad containing the R-1, however, must be on top of the test pad containing R-2. This process can be utilized for any of the above test methods as long as the indicator yields a color response in the visible color spectrum.

In addition to the final method mentioned above for analysis of analytes of interest in fluids. Biological fluids for example could be used. Serum will be used in this example with an enzyme-labeled nucleounit to one or more of the analytes of interest markers. Again, many different enzymes or co-enzymes can be utilized for this purpose. For example the reagent R-1 would have the following components: glucose-6-phosphate dehydrogenase conjugated to a to homocysteine (forming a labeled homocysteine) and a nucleounit specific to homocysteine. This solution forms the active portion of R-1, and is mixed first with sample. If free homocysteine is present in the sample, it will compete with labeled homocysteine. The more free homocysteine in the sample the more labeled homocysteine will not be bound by the nucleounit specific for homocysteine. The reduction in binding between the labeled homocysteine and the nucleounit is directly proportional to the concentration of free homocysteine in the serum sample and can be measured by a detectable means. The R-2 reagent would of course contain the substrate glucose-6-phosphate and NAD. As the free homocysteine in the sample competes with the labeled homocysteine in solution the more glucose-6-phosphate dehydrogenase will interact with glucose-6-phosphate. As this reaction takes place NAD is reduced to NADH which can be measured spectrophotometrically. Additionally, the reagent R-2 could contain NBT (nitro blue tetrazolium) which will change color when NAD is reduced to NADH and become detectable visibly or by other means.

Please note this method can be adapted to dry chemistry dipstick technology. First, the solid paper matrix is immersed in the R-2 reagent, and finally in the R-1 reagent. Alternatively, two distinct paper matrices, or test pads could be used (one for each immersion solution), and a “sandwich” made by stacking the two pads, one on top of the other. The paper pad containing the R-1, however, must be on top of the test pad containing R-2. This process can be utilized for any of the above test methods as long as the indicator yields a color response in the visible color spectrum.

The present invention encompasses a method that can utilize several different techniques. The first technique employs a liquid reagent compatible with most chemistry analyzers currently used for clinical chemistry testing to quantitate the amount of analytes of interest present in each sample. In addition, this liquid reagent can also be used in classical wet chemistry and spectroscopy techniques. The second technique employs the dry chemistry dipstick (DCD) method. A third technique employs a combination of DCD and thin layer chromatography called a lateral flow device (LFD). Utilization of the liquid reagent with the automated chemistry analyzer facilitates high volume testing (i.e. thousands per hour) and permits testing for analytes of interest while simultaneously running routine chemistries on the same sample using the same analyzer. In the case of testing on a spot urine sample, the additional tests would include creatinine, cystatin C and/or other “normalizing” factors such as osmolality, or specific gravity. The current analyzers can also perform the math required to yield a normalized analyte of interest value (e.g. GHB quantitation/creatinine concentration (ratio) or GHB quantitation/cystatin C concentration (ratio)). The resulting report generated includes GHB and GHB ratio results and all the routine chemistry as requested by the physician. This unified report allows the physician to evaluate test results and report findings rapidly and efficiently. It may also facilitate further testing, and/or prevent costly additional tests.

The automated analysis procedure encompasses the following automated method(s) for the measurement of analytes of interest on an unknown sample of urine (or other biological sample including serum, whole blood, cerebral spinal fluid, gastric fluid, sweat extracts hair homogenates, and saliva). A method(s) to determine analytes of interest by measuring the concentration of analytes of interest in a test specimen, said test method comprising the steps of placing the reagent composition(s), R-1 and R-2, in the reagent compartment of the chemistry autoanalyzer, aliquoting samples, calibrators, and controls into sample cups and placing them on the chemistry autoanalyzer, transferring an aliquot of each sample, calibrator, and control into single, discrete cuvettes mounted within the chemistry autoanalyzer, aliquoting a specified volume of the first reagent composition, R-1, into each cuvette and mixing, incubating the reaction mixture for a specified time interval, aliquoting a specified volume of the second reagent composition, R-2 (if required), into each cuvette and mixing, incubating the reaction mixture for a specified time interval, measuring and recording absorbance values of the reaction mixtures with the chemistry autoanalyzer's spectrophotometer at specified wavelength (from 340 to 800 mn) and at preprogrammed time intervals, and comparing absorbance values of samples and controls to those of the calibrators in the form of a standard curve thereby quantitating the analyte of interest present.

The other techniques, dry chemistry dipsticks (DCDs), and lateral flow devices (LFDs) are solid phase assays that use an absorbent medium such as paper which has been impregnated with the chemical formulations needed to perform the assay. To summarize more specifically the foregoing dry chemistry test strip (DCD) method for the measurement of analytes of interest concentration(s) in a urine sample(s), said test method comprising the steps of preparing a test means by successively impregnating a carrier matrix with reagent solutions, drying said test means, dipping completed test means into test sample, and determining the quantity of analytes of interest in said test sample by comparing the relative intensity of the color produced by the reaction to a color chart with color blocks referenced to specific concentrations of the analyte of interest. To summarize more specifically the foregoing lateral flow test device (LFD) method to determine a particular analyte of interest by measurement of the analyte of interest concentration in a urine sample, said test method comprising the steps of preparing a test means by successively impregnating a carrier matrix with reagent solutions at specific target locations on said test means, drying said test means, dipping into or depositing an adequate amount of test sample to the device at the starting point of the analysis, allowing sufficient time to complete the migration of sample to the end point of the analysis, and determining the presence or absence of the particular analyte of interest in said test sample by comparing the lines produced by the reaction to a result chart for concentrations of the analyte of interest. Ease of use and rapid results obtained mark the unique utility of these testing techniques. In addition, very little technical expertise is required to perform these types of assays (i.e. DCDs and LFDs).

A thorough search of the literature reveals no relative art resembling this technology; therefore, this invention is clearly a novel creation, and is not obvious to anyone skilled in the art of determination of analytes of interest using nucleounits specific for the targets of interest.

### DETAILED DESCRIPTION OF THE INVENTION

The instant invention provides test strips (i.e. DCDs and LFDs), dipstick/lateral flow hybrid (which can be known as the “DLFH” device), or automated liquid chemistries for the detection of analytes of interest in biological or other samples through the use of nucleounits resulting in the concomitant determination of a specific analyte of interest with a hitherto unachievable high level of ease of use and sensitivity. Essentially, the present invention comprises test strips (carrier dependent, solid phase) or liquid reagents (carrier independent, aqueous phase) designed to detect analytes of interest which do not require the use of antibodies which is a prerequisite of the prior art.

The DCD, LFD, or DLFH rapid test strips and the aqueous, liquid chemistry reagents in conjunction with the nucleounits can consist of an indicator(s) conjugated or attached otherwise or used in conjunction with the nucleounits such as (colorimetric, enzymatic, fluorescence, turbidimetric, radioimmuno, antibody, ion-exchange, or ionic) that are specific for analytes of interest. That is to say the “analyte(s) of interest” are targets for the nucleounit(s) and can for example be glucose in urine or the nucleounit target can be used to detect virulent disease causing viruses such as HIV, or proteins, viruses, drugs of abuse, drug metabolites, and therapeutic drugs such as the following, but not limited to the following; cocaine, opiates, gamma-hydroxybutyric acid, cannabinoids, benzodiazepines, acetaminophen, amikacin, aminocaproic acid, amitriptyline, amobarbital, amphetamine, bromide, caffeine, carbamazepine, carbenicillin, chloral hydrate, chloramphenicol, chlordiazepoxide, chlorpromazine, cimetidine, clonazepam, clonidine, clorazepate, cocaine, cocaine metabolites, ethanol, methanol, or other forms of alcohol, codeine, cyclosporine, desipramine, dexamethsone, diazepam, digoxin, diphenylhydantoin, disopyramide, doxepin, ephedrine, ethchlorvynol, ethosuximide, fenoprofen, flecainide, flurazepam, gentamicin, glutethimide, hydromorphone, ibuprofen, imipramine, isoniazid, kanamycin, lidocaine, lithium, lorazepam, lysergic acid, meperidine, meprobamate, methadone, methamphetamine, methaqualone, methotrexate, methsuximide, methyldopa, methyprylon, morphine, n-acetylprocainamide, netilmicin, nortriptyline, oxazepam, oxycodone, paraldehyde, paraquat, pentazocine, pentobarbital, phenacetin, phencyclidine, Phenobarbital, phensuximide, phenylbutazone, phenylpropanolamine, phenytoin, primidone, procainamide, propoxyphene, propranolol, protripyline, quinidine, salicylates, secobarbital, theophylline, thiocynate, thiopental, thioridazine, tobramycin, tolbutamide, valproic acid, vancomycin, or warfarin, . . . etc., or clinical chemistry targets such as the following, but not limited to the following; cholesterol, triglycerides, glucose, adrenocortocotropic hormone, alanine, alanine aminotransferase, albumin, aldolase, aldosterone, amylase, amyloid-associated protein, androstenedione, angiotenesis, antidiuretic hormone, antithrombin, antitrypsin, apolipoprotein, ascorbic acid, bile acids, bilirubin, c-peptide, calcitonin, calcium, cancer antigen 125, carboxyhemoglobin, carotene, catecholamines, cholic acid, cholyglycine, chromium, chymotrypsin, complement components, coproporhyrin, corticobinding globulin, corticosterone, cortisol, c-peptide, c-reactive protein, creatine, creatinine, creatine kinase, cyclic AMP, cystine, cysteine, dehydroepiandrosterone, dehydroepiandrosterone sulfate, deoxycholic acid, 11-deoxycortocosterone, 11-deoxycortisol, dihydrotestosterone, estradiol, estriol, estrogen, estrone, fecal fat, fatty acids, ferritin, fetoprotein, fibrinogen, folate, follicle stimulating hormone, thyroxine, triiodothyronine, fructose, fructosamine, galactose, gastric acid, gastrin, glucagons, glucose-6-phosphate, glutamine, glutamyltransferase (GGT), glutathione, hemoglobin, glycerol, glycine, glycolic acid, gold, growth hormone, haptoglobin, high-density lipoproteins, hemopexin, hemocystein, homocysteine, homogentisic acid, homovanillic acid, hydrogen sulfide, 17-hydroxycortocosteriods, 5-hydroxyindoleacetic acid, 17-hydroxyprogesterone, hydroxyproline, immunoglobins, insulin, iron, isocitrate dehydrogenase, isoleucine, 17-ketogenic steroids, ketone bodies, lactate, lactate dehydrogenase, lactose, LDL-cholesterol, lecithin, leucine, leukocyte, lipase, lipoproteins, lutropin, lysozyme, macroamylase, magnesium, melanin, metanephrine, methionine, metyrapone, microsomal antibodies, antibodies, molybdenum, mucoploysaccharide, myelin basic protein, myoglobin, methemoglobin, niacin, nickel, nitrite, nitrogen, nonprotein nitrogen, normetanephrine, blood, orosomucoid, oxalate, oxytocin, pancreatice polypeptide, pantothenic acid, parathyroid hormone, pentachloropehnol, pentoses, pepsinogen, phenols, phenolsulfonphthalein, phenylalanine, acid phosphatase, alkaline phosphatase, phosphofructokinase, phospholipids, placental lactogen, plasminogen, porphobilinogen, prealbumin, pregnanediol, pregnanetriol, pregnenolone, progesterone, porinsulin, properdin, prostaglandins, prostate-specific antigen, portoporphyrin, pseudocholinesterase, pyruvic acid, renin, reverse triiodothyromine, rheumatoid factor, riboflavin, secretin, selenium, serotonin, somatomedin c, sucrose, testosterone, tetrahydrocortisol, tetrehydrodeoxycortisol, thallium, thyroglobin, thyroid antibodies, thyroxine binding globulin, thyroxine, transcortin, transferring, transketolase, transthyretin, thyrotropin-releasing hormone, triglycerides, triiodothryonine, tyrosine, urea, urea nitrogen, uric acid, uricase, urobilinogen, uroporphyrin, valine, vanillymandelic acid, vasoactive intestinal polypetide, human chorionic gonadotropin, mass creatinine kinase, vitamins, xylose, or zinc, toxins or poisons such as the following, but not limited to the following; cyanide, formaldehyde, ethylene glycol, lead, mercury, xylene, and immunochemistry targets and infectious disease targets such as the following but not limited to the following: human immumodeficiency virus (HIV), cytomegalovirus (CMV) IgG, cytomegalovirus (CMV) IgM, herpes simplex virus (types 1 and 2) IgG, rubella IgG, rubella, IgM, toxoplasma IgG, toxoplasma IgM, amebiasis, Epstein-barr early antigen, Epstein-barr EBNA IgG, Epstein-barr VCA IgG, Epstein-barr VCA IgM, helicobacter pylori-IgG, legionella IgG/IgM/IgA, mycoplasma IgG, mycoplasma IgM, varicella zoster virus (VZV), or autoimmune diseases such as the following but not limited to the following; antinuclear antibodies (ANA), antineutrophil cytoplasmic antibodies (ANCA), anti-cardiolipin, anti-dsDNA, anti-Jo-1, anti-Scl-70, anti-Sm (Smith antigen), anti-Sm/RNP, anti-SS-A/Ro, anti-SS-B/La, extractable nuclear antigen (ENA), myeloperoxidase IgG, proteinase-3 IgG, and Rheumatoid Factor. The measureable test means of the present device produces a visual, spectrophotometric, turbidimetric, fluorescence, or reflectance result by the combination of the nucleounit with its target resulting in the production of a measurable response.

One novel aspect of this new art eliminates the need to use the prior art methods use of antibodies (thereby eliminating the use and abuse of animals for the production of antibodies as currently required by the prior art) and other antiquated techniques such as HPLC and ELISA methods, which are tremendously tedious an time consuming. The present art's ability to increases sensitivity and accuracy while not using antibodies is remarkable and produces unexpected results. The results achieved by the present art use of the nucloeunit in conjunction with the reagent systems as taught in this disclosure are new, unexpected, superior, disproportionate, unsuggested, unusual, critical, and surprising. The problems of the prior arts use of animals and the unstable characteristics of antibodies in comparison to the present arts ability to provide all of the advantages of the prior with improvements in specificity, accuracy, sensitivity, and stability without the use of animals is absolutely novel and solves the many manifold problems of the prior art. The present arts test means that is applicable to DCDs, LFDs, and aqueous, liquid reagents compatible with automated analyzers is a tremendous advancement in the art that will significantly lower the cost of the testing and improve results. The present device has no such requirement.

This new art's ability to analyze test samples using DCD or LFD technology can not be stressed enough. This one important leap in technology allows the physician at his or her office, and the patient at home to test for analytes of interest that currently requires the use of antibodies without a laboratory. This will have a tremendous impact for thousands of doctors offices by providing an inexpensive and accurate method for clinical diagnostics in addition the present art enables the user to purchase the device for OTC (over the counter) use. One of the fastest growing markets is the (point of care). This allows individuals to use the products at the point of collection or at home without the use of expensive equipment.

The detection methods of the present device constitute the heart of the analytical response provided by it, and is comprised of one or more reagent compositions containing an nucleounit responsive to the analyte of interest producing a detectable response. These test means (nucleounits) are thus able to interact with the target analyte of interest in a test sample, and yield a detectable response. The response can be in the form of the appearance or disappearance of a color or line, or the changing of one color to another. Said measurable response may also be evidenced by a change in the amount of light reflected or absorbed during the reaction of interest. The analytical arts are replete with examples of these types of detectable responses. Thus the reagent composition of the present device constitutes the heart of the analytical process, and in the broadest sense includes one or more reagent compositions composed of chemical compounds responsive to the analyte of interest thereby producing some detectable manifestation of the presence of said analyte of interest (i.e., glucose molecule, AIDS virus, selected bone antigens, etc.). The response can be in the form of the appearance, disappearance, or change in intensity of one or more colors in the ultra violet or visible spectrum. Such changes can be measured with a spectrophotometer or calorimeter using direct absorbance or reflectance. In the case of the visible spectrum, the human eye can also determine the color changes or the appearance of a colored line. Consequently, according to the present invention, there is provided a method(s) for determining the analyte of interest by the measurement of the nucleounit(s) interaction with the analyte of interest in an unknown test sample, said test method being composed of a buffer and an indicator reagent that produces a color change, or a change in the absorbance or intensity of the color in the UV or visible spectrum in the presence or absence of the analyte of interest.

Those skilled in the prior art could not have been foreseen the development of this new art and the tremendous advancement it represents in the field of clinical diagnostics, treatment, and animal rights.

All of the method(s) for the measurement of analytes of interest on an unknown samples using nucleounits can be performed on samples composed of biological matrices such as urine, serum, whole blood, cerebral spinal fluid, gastric fluid, sweat extracts hair homogenates, and saliva. The test sample can be made up any fluid or fluids such as beverages such water, soft drinks, beer, or mixed drinks to include liquid drinks from a bar that might be contaminated with GHB.

The instant invention is comprised of a reagent containing an enzyme and/or an antibody and/or an indicator, and buffer. Optional components include a substrate, surfactant (i.e. wetting agent), and compounds for removal of interfering substances. A few substances which remove sample matrix interferences include mono, di, tri, and tetra sodium salts of EDTA or EGTA. One or more of these interference-removing compounds can be mixed with the test specimen as part of the R-1 of the reagent composition. Note this instant invention will be referred to hereafter as nucleounit assay reagent system. Buffering of the reactants acts to stabilize pH. It is well known in the art that most reactions have an optimum pH range, and an ideal buffer should be selected on that basis. This new art formula will require appropriate buffering. Suitable buffers may include any of the following (referred to here by their commonly used acronyms and chemical names): citrate, borate, borax, sodium tetraborate decahydrate, sodium perchlorate, sodium chlorate, sodium carbonate, TRIS (Tris[hydroxymethyl]aminomethane), MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), PIPES (PiperazineN-N′-bis[2-ethanesulfonic acid)]; 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), TES (N-tris[Hydroxymethyl]methyl-2-aminomethanesulfonic acid; 2[2-Hysroxy-1,1-bis(hydroxymethyl)-ethyl]amino)ethanesulfonic acid), HEPES (N-[2-Hydroxyethyl]piperazine-N′-[2-ethanesulfonic acid]), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine; N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

Manufacture of the dry chemistry dipsticks may require the addition of thickeners as taught in the art. Some compounds commonly used for this purpose include: polyvinylpyrrolidone, algin, carragenin, casein, albumin, methylcellulose, and gelatin. The typical range of concentration for these thickeners is about 0.5 to 5.0 g. per 100 ml. Wetting agents or surfactants are also typically used in dry chemistry. For dry chemistry applications, wetting agents aid in even distribution of the chemicals and promote even color development. Acceptable wetting agents can be hydrophilic polymers, or cationic, anionic, amphoteric, or nonionic species. Some commonly used wetting agents include sodium dodecyl-benzene sulphonate, sodium lauryl sulphate, benzalkonium chloride, N-lauroylsarcosine sodium salt, Brij-35, Tween 20, Triton X-100, dioctyl sodium sulphosuccinate, and polyethylene glycol 6000. Wetting agents can be added to dipstick impregnation solutions in amounts of 0.5% to 5.0%, and 0.1% to 1.0% in liquid reagents.

Color enhancers may be used such as sucrose, lactose, glucose or other compounds. Color enhancement can be defined as intensification and/or alteration in some manner the color that is produced by the reaction to improve the measurement of the detectable response.

The production of dry chemistry test strips for the present invention can utilize any form of absorbent, solid phase carrier including filter paper, cellulose or synthetic resin fleeces in conjunction with liquid solutions of reagent compositions in volatile solvents. This can be carried out in one or more impregnation steps. Each impregnation may contain one or more of the chemical compounds making up the assay reagent composition; the exact procedure is dictated by the inter-reactivity of the assay constituents and the order in which they may have to react with the analyte of interest.

In the case of the DLFH or LFD of the present invention it can utilize any form of absorbent, solid phase carrier that is capable of transporting a fluid. These can include filter paper, cellulose or synthetic resins. More specifically, the lateral flow material can include cellulose, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

After impregnation, the dipsticks are dried, cut into strips, glued to a support structure (usually a flexible, flat, plastic stick made up of polystyrene, vinyl polypropylene, and polyester or other suitable support material) as part of a “sandwich” composed of the handle, test pad, and a synthetic resin film and/or a fine-mesh material in the manner described in German Pat. No, 2,118,455. In addition, the instant invention may be combined with the water-stable film as taught in U.S. Pat. No. 3,530,957 to produce a dipstick in which the excess sample fluid can be wiped off in order to improve the accuracy and precision of the results.

In the case of the liquid reagent, the chemical composition is dissolved in water, and the pH of the solution(s) is adjusted. In some circumstances, the analysis may require a two-part reagent system, or two solutions. The analysis proceeds by placing reagent and samples on the automated chemistry analyzer; samples, standards and controls are then pipetted from the sample cups into reaction cuvettes, mixed with reagent which is added to the cuvettes, and absorbance readings (taken at a specified time interval using a preprogrammed wavelength) are taken, stored, and compared to known standard values to quantitate the amount of analyte of interest in each unknown sample.

In the case of DCD technology, the manufacturing process includes impregnating onto an absorbent, solid carrier (e.g. paper) the chemical constituents which have been dissolved in a liquid solvent, evaporating the solvent, and mounting this “reaction paper” on a sturdy plastic “handle”; this device is then dipped into the test sample, withdrawn, and the visible color produced is observed and compared to a chart which relates specific colors or shades of the same color to a range of concentrations of the target analyte. Note the absorbent paper can also act as the support handle.

In the case of LFD technology, the manufacturing process includes impregnating onto an absorbent, solid carrier (e.g. paper) the chemical constituents which have been dissolved in a liquid solvent, evaporating the solvent, and mounting this “reaction paper” on a solid support which can encapsulate the LFD test pad except for the point of application of sample, and any areas in which results (e.g. colors or lines) are to be observed; sample is then placed on the device at the bottom or starting point for the assay, and after the simple has migrated to the top of the test pad, the appearance of lines on the device is compared to the result chart and results are recorded. Note, the test pad must be an absorbent wicking material that permits migration of sample up the solid absorbent test pad and allows analytes and reactants to interact at specific binding sites along the test pad.

The following is a brief explanation of the LFD technology of this invention, and will be described in detail in the following examples. This example is purely illustrative and this art is not limited to this description.

For example a LFD device is made to detect the presence of hepatitis C virus (HCV) in urine using the novel nucleounit technology. This LFD device is approximately 5 mm wide by 70 mm long. The absorbent material is cut to fit these dimensions. For this example the device will use HVC cutoffs of 10.0 fmol/L HCV (the presence of any HCV in urine is considered a positive). The starting point or origin at which the sample is placed on the test device is 5 mm from one end of the strip, and 30 mm from this origin a buffered solution containing nucleounit(s) specific for colored particle(s) is bound to the test strip 35 mm from the bottom edge of said test pad in a line approximately 1 mm wide by 5 mm long thereby extending from one side of the device to the other side forming the C, “control line”. The appearance of a colored line here after assay is complete will indicate that the lateral flow device worked properly (i.e. the sample migrated to an acceptable RF value beyond the A or assay line and the binding capacity of the nucleounit specific for colored particles are reactive and nothing in the sample has adversely affected the test's reactants). A second buffered solution consisting of blue colored particles bound (i.e. irreversibly coupled, conjugated, or covalently linked) to 100 fmol/L of HCV nucleounit specific for HCV, and 10 fmol/L of blue colored are applied to the strip approximately 5 mm from the starting point (or 10 mm from the lower edge of the test strip) in a concentration as to make certain that assay and control lines both form solid visual lines to achieve effective results. A third buffered solution of HCV nucleounit(s) specific for HCV is coupled (bound) to the strip at approximately 10 mm from the starting point of the strip (or 15 mm from the lower edge of the test strip) forming the A (assay) line approximately 1 mm wide by 5 mm long thereby extending from one side of the device to the other side forming the immobilized, coupled HCV nucleounit line. This line (the assay line) will react with the bound HCV that been captured by the blue colored particles containing the HCV specific nucleounit(s). A solid plastic case may be used to conceal and protect all of the device except for three “windows”; one for sample application at the origin, a second at the A, assay line, and a third at the C, control line.

If the sample is positive, with a concentration of 10.0 fmol/L HCV or more the following occurs. A drop of urine (approximately 50 uL) is applied at the starting point or origin of the strip. The urine then migrates to the opposite or terminal end of the strip. The free HCV present in the urine binds all of the HCV nucleounits conjugated to the blue colored particles, these particles along with the free colored particles migrate with the urine toward the terminal end of the strip away from the starting point. The HCV nucleounit colored complexes will bind to the line at the 10 mm “A” assay window because the HCV nucleounit(s) that have been immobilized on the strip with capture the particles and form a solid line (the lack of a solid line would indicate a negative sample for HCV). The unbound free migrating blue colored particle complexes (which are the control particles), continue migrating up the device until reaching the line of bound nucleounits specific for the colored particles at the “C” or control window forming a solid (complete) blue “control line” consisting of immobilized nucleounit(s) specific for the colored particles and the bound colored particles.

If the sample is negative, with no HCV present the following will occur. A drop of urine (approximately 50 uL) is applied at the starting point or origin of the strip. The urine then migrates to the opposite or terminal end of the strip. No binding of the HCV nucleounits conjugated to the blue colored particles occurs, these particles along with the free colored particles migrate with the urine toward the terminal end of the strip away from the starting point. The unbound free migrating blue colored particle complexes (which are the control particles), and the HCV nucleounit particles continue migrating up the device until reaching the line of bound nucleounits specific for the colored particles at the “C” or control window forming a solid (complete) blue “control line” consisting of immobilized nucleounit(s) specific for the colored particles and the bound colored particles.

This brief description of the present art illustrates a completely enabled device that would allow a physician, patient, and/or technician to quickly and easily determine the HCV value or HCV exposure of the patient. The HCV normal value for this example is less than 10 fmol/L HCV. A value of equal to or greater than 10 fmol/L of HCV indicates the patient has been exposed to HCV. Please note that these concentrations for the above example are merely illustrative. If the analysis is performed on a 24-hour urine collection, no further analysis is required. Proper 24-hour urine collections are difficult and inconvenient for the patient, however, the above test can also be performed using a random specimen. Consequently, a novel addition to further improve the ease of use and the accuracy of the present device requires an additional assay on the same random or spot urine used for the HCV assay. This additional assay is for creatinine or cystatin C. These analyte values can be used to “normalize” or correct the HCV result for the amount of water present in the sample. Water content of a random urine sample is affected by the diurnal variations, diet, diuretics (e.g. caffeine, sugar) and short-term fluid consumption (water consumed over the previous 2 to 3 hours). The amount of creatinine or cystatin C excreted by a normal, healthy individual is relatively consistent from day to day, and hour to hour; any HCV if present would also be excreted at a consistent rate from hour to hour. Creatinine and Cystatin C are, therefore, ideal for adjusting or normalizing the amount of HCV found in a random urine.

Obviously if the creatinine or cystatin C concentration is high the subject has consumed very little water over the previous few hours, and the HCV value will be elevated; if the subject has consumed a large volume of water just prior to testing, the creatinine or cystatin C value will be low and the HCV concentration will also be depressed.

The following formula may be used to adjust the HCV value according to the creatinine or cystatin C concentration. In this example creatinine will be used instead of cystatin C or some other steady state marker. This example requires multiplication of the marker value by the volume of urine (50 uL in the above example) divided by the creatinine concentration of the sample. This yields a normalized HCV value for a random sample. The method of measuring creatinine in urine by LFD is hitherto unknown in the art until the present device and examples of this methodology will follow. If analysis is being performed via automated chemistry, a number of methods are currently available. And finally this present art incorporates the unique invention of HCV assay with the use of a ratio of HCV to creatinine. This is the value of the HCV divided by the concentration of creatinine. This ratio provides the most convenient way to normalize the HCV value and allow the user, even an untrained one, to obtain a corrected HCV value.

Two additives are typically included in the production of dry chemistry test strips. These are thickening agents and wetting agents. The latter is also an integral part of liquid reagent compositions. The relatively large amount of water-soluble substances present in the recommended formulations tend to promote “bleeding” (i.e. seeping out of the test pad upon re-wetting with test specimen or additional reactants in successive immersions); thickening agents prevent or limit this phenomenon. Some typical compounds used for this purpose include polyvinylpyrrolidone, algin, carrageenin, casein, albumin, methyl cellulose, and gelatin in concentrations ranging from 0.5 to 5 g. per 100 ml. Wetting agents are also typically recommended to aid in even distribution of reactants and even color development. Compounds typically used for this purpose include long chain organic sulphates or sulphonates (e.g. Brij-35, Tween 20, Triton X-100, dioctyl sodium sulphosuccinate, and sodium lauryl sulphate). Wetting agents are typically added to impregnation solutions in amounts from 0.5 to 5 percent. In liquid reagents to be used on automated analyzers, wetting agents improve solubility of reactants, improve flow characteristics through the instrument's tubing, increase distribution and development of color, and reduce formation of bubbles in solution.

Again, the production of the test strips according to the present invention requires an absorbent carrier which may be any of the following: filter paper, cellulose, lateral flow paper/material, and synthetic resin fleeces. Immersion solutions may be aqueous or volatile, organic solvents. The order of application and number of immersion solutions will vary according to the specific assay reaction to be utilized (see examples in this section).

After drying, the test's absorbent material is cut into strips and may be sealed between a synthetic resin film and a fine-mesh material in the manner described in German Pat. No, 2,118,455, and further delineated in U.S. Pat. No. 3,530,957. This technique permits the surface of the test strip to be wiped dry after dipping in the test sample to improve readability of the test pads.

The manufacture of the nucleounits which are specific for analytes of interest are made as follows: The manufacturing starts with a chemically synthetic random sequence of nucleotide libraries made up of 20 or more nucleotides bound in random sequences containing approximately 10¹⁰ to 10²⁰ individual nucleounits. Each nucleounit in the library is designed to contain a contiguous randomized region flanked by two fixed sequences of known value or content each. The section of the nucleounit that contains the contiguous random region is synthesized upon delivery to the mixture of nucleounits. The mixture contains phosphoramidites made up of all five building blocks (nucleotides): A, G, C, U and T (A=adenine, G=guanine, C=cytosine, U=uracil, and T=thymine). To obtain an unbiased library with equal representation of the phosphoramidites, the ratios of the four nucleotides used in the mixture are adjusted based upon coupling efficiencies of the individual molecules that can be chemically bound as a unit. Fixed sequences of the nucleounits are used for primer binding sites in enzymatic amplification of individual nucleounit sequences. The random sequence nucleounit libraries are exposed to the analyte of interest under specific buffer conditions. The nucleounits that bind the analyte of interest are separated from the unbound nucleounits by a suitable partitioning technique. This population of nucleounits recovered from the analyte of interest (mixture of nucleounit candidates) represents a mixture containing both high-affinity and low-affinity binding nucleounits to the analyte of interest. Further screening is required to eliminate the sequences with low-affinity binding. Individual sequences are amplified for the next round of selection. The nucleounit candidate mixture is amplified directly by PCR (polymerase chain reaction). The enrichment of the high-affinity nucleounits is at the expense of the low-affinity binders requiring several replications of the process carried out under increasingly difficult conditions which refine the selection process for the nucleounit with the greatest binding capacity. Once the affinity saturation is observed in an enriched library of nucleounits, the corresponding PCR products are used for cloning and sequencing. For example manufacturing of the nucleounits specific for myoglobin are made as follows: The manufacturing starts with a chemically synthetic random sequence of nucleotide libraries made up of 20 or more nucleotides bound in random sequences containing approximately 10¹⁰ to 10²⁰ individual nucleounits. Each nucleounit in the library is designed to contain a contiguous randomized region flanked by two fixed sequences of known value or content each. The section of the nucleounit that contains the contiguous random region is synthesized upon delivery to the mixture of nucleounits. The mixture contains phosphoramidites made up of all five building blocks (nucleotides): A, G, C, U and T (A=adenine, G=guanine, C=cytosine, U=uracil, and T-thymine). To obtain an unbiased library with equal representation of the phosphoramidites, the ratios of the four nucleotides in the mixture are adjusted based upon coupling efficiencies of the individual molecules that can be chemically bound as a unit. Fixed sequences of the nucleounits are used for primer binding sites in enzymatic amplification of individual nucleounit sequences. The random sequence nucleounit libraries are exposed to myoglobin using a 0.01 M TRIS buffer at a pH of 7.0. The nucleounits that bind myoglobin are separated from the unbound nucleounits by partitioning. This population of nucleounits recovered from the myoglobin (mixture of nucleounit candidates specific to myoglobin) represents a mixture containing both high-affinity and low-affinity binding nucleounits to myoglobin. Further screening is required to eliminate the sequences with low-affinity binding. Individual sequences are amplified for the next round of selection. The nucleounit candidate mixture is amplified directly by PCR (polymerase chain reaction). The enrichment of the high-affinity nucleounits is at the expense of the low-affinity binders requiring several replications of the process carried out under increasingly difficult conditions which refine the selection process for the nucleounit with the greatest binding capacity. Once the affinity saturation is observed in an enriched library of nucleounits, the corresponding PCR products are used for cloning and sequencing. The refined nucleounits can now be used to target myoglobin in LFD, DCD, DLFH, and liquid devices designed specifically for this purpose. Examples of manufacturing techniques used to produce such devices will follow in detail.

The following are examples of groups of indicator compounds that will function in dry and liquid chemistry assays used in conjunction with analyte specific nucleounits:

I. Indicators

1. Color indicators which produce color by oxidation/reduction

2. UV-visible color indicator bound to Enzyme-specific substrate

3. Enzymatic indicator

4. Fluorescence indicator

5. Turbidimetric indicator composed of aggregate-forming microparticles

6. Ionic indicator

7. UV and visible indicators bound to specific antigen to analyte of interest.

8. UV and visible indicators bound to specific antibody to analyte of interest.

9. Antibodies and Antigens that react with the nucleounits.

Consequently, according to the present invention, an assay means for the determination of analytes of interest via the use of nucleounits in biological matrices or other specimens, may comprise either a test strip composed of a solid, carrier matrix in the form of absorbent paper impregnated with a reaction mixture containing an indicator compound of the general formula (I), dried, and attached to a sturdy handle to form a dry chemistry dipstick (DCD), lateral flow device (LFD), or the dipstick/lateral flow hybrid (which can be known as the “DLFH” device), or a liquid reagent composed of an aqueous solution containing an indicator compound of the general formula (I) that is compatible with most general chemistry auto-analyzers.

Development of the present invention and the concomitant extraordinary increase in utility of it is not obvious in view of the prior art. The present invention targets biological matrices and other fluids as previously described.

The following examples are provided to further illustrate the inventive aspects of the present discovery, and to further describe preferred embodiments. As such, they are intended as being merely illustrative, and are not to be construed as limiting the scope of the claims appended hereto.

### EXAMPLE 1

The following procedure is a method for manufacturing a dry chemistry lateral flow test strip for the determination of cytomegalovirus (CMV) viral exposure by measurement of a sample's anti-CMV concentration; in this example the manufactured nucleounit target is anti-CMV. Note CMV infection of normal children and adults usually results in asymptomatic disease, but infection can cause hepatitis, pneumonia or a mononucleosis-like illness. If acquired in utero or at birth, CMV infection results in congenital abnormalities.

Absorbent material is successively impregnated with the following solutions and dried at 25 degree C.:

Solution 1

0.025 M Phosphate buffer pH 6.9

150 fmol/L nucleounit to colored particles

Solution 2

0.05 M Phosphate buffer pH 6.9

10 fmol/L nucleounit to CMV antibody conjugated to red microparticles

50 fmol/L red microparticles

Solution 3

0.025 M Phosphate pH 6.9

50 fmol/L anti-CMV

In this example, the lateral flow device is prepared in accordance with the instant invention. The lateral flow device is comprised of a paper carrier matrix impregnated with the compositions of solutions 1, 2, and 3 above. Note that said concentrations of any of the above constituents can be varied to suit the lateral flow/dipstick device format (e.g. dependent upon paper type, and inclusion of semi-permeable membranes or other innovations utilized in dry chemistry technology). Production of this test device is carried out using the following procedure. The test device made up of a solid support which includes an absorbent material capable of transporting a liquid by capillary action or wicking (e.g. nitrocellulose 5.0 u, S&S brand) in this example having dimensions of 5 mm by 70 mm and can be backed by or in contact with strips of glass fiber (e.g. Whatman GF/A) to aid in controlling the wicking action. In this example, the device uses an CMV cutoff of 10 mol/L anti-CMV in serum.

The starting point or origin at which the sample is placed on the test device is 5 mm from one end of the strip, and 30 mm from this origin solution 1 (a buffered solution containing nucleounit to colored particles) is bound to the test strip 35 mm from the bottom edge of said test pad in a line approximately 1 mm wide by 5 mm long thereby extending from one side of the device to the other side forming the C, “control line”. The appearance of a colored line here after assay is complete will indicate that the lateral flow device worked properly (i.e. the sample migrated to an acceptable RF value beyond the A or assay line due to the red particles binding to the nucleounits targeted to the red particles and the binding capacity of the anti-CMV and the bound nucleounit to CMV antibody conjugated to red microparticles are reactive and nothing in the sample has adversely affected the test's reactants). A second buffered solution (solution 2) consisting of red colored particles bound (i.e. irreversibly coupled, conjugated, or covalently linked) to nucleounits to CMV antibody and free red particles are applied to the strip approximately 5 mm from the starting point (or 10 mm from the lower edge of the test strip) in a concentration as to make certain that assay and control lines both form solid visual lines to achieve effective results. A third buffered solution of anti-CMV is coupled (bound) to the strip at approximately 10 mm from the starting point of the strip (or 15 mm from the lower edge of the test strip) forming the A (assay) line approximately 1 mm wide by 5 mm long thereby extending from one side of the device to the other side forming the immobilized, coupled anti-CMV line. The starting point or point of origin from where the fluid (in this example serum) starts is at the 5 mm point from the end of the strip, 30 mm from the control line. This is where three drops of the sample are applied. A solid case may be used to conceal and protect all of the device except for three “windows”; one for sample application at the origin, a second at the A, assay line, and a third at the C, control line. This case may be composed of plastic, wood, cardboard, or other suitable material.

If the sample is positive, with a concentration of 10 fmol/L anti-CMV or more the following occurs. Three drops of serum (approximately 100 uL) are applied at the starting point or origin of the strip. The serum then migrates to the opposite or terminal end of the strip. The free anti-CMV present (in a concentration of 10 fmol/L or greater of anti-HIV) in the serum binds all of the red particles bound with the nucleounit targeted to the CMV antibody (10 fmol/L) and these conjugated particles complexes containing CMV antibody bound to the nucleounit targeted to CMV antibody which are attached to red particles migrate with the serum toward the terminal end of the strip away from the starting point. These colored complexes will not bind to the line of anti-CMV bound at the 10 mm “A” line or assay window because all of the particles with nucleounits to CMV antibodies are already bound up by the free anti-CMV from the sample. The migrating red colored particle complexes and the free red particles continue migrating up the device until reaching the line of bound nucleounits targeted to the red particles at the “C” or control window. The anti-CMV-nucleounit red particle complexes and the red colored particles then bind to this line forming a solid (complete) red “control line” consisting of both types of red particles (bound and unbound).

If the sample is negative, with a concentration of less than 10 fmol/L of anti-CMV present, the following occurs. The sample is applied then the free (unbound) nucleounit to CMV antibody conjugated to red microparticles complexes migrate up to the “A” assay line and bind to the anti-CMV conjugated to the test strip at that location thereby forming a solid (complete) red line assay line. The control particles (i.e. red particles) will keep migrating to the 35 mm “C” control line and form a solid red line to indicate the assay worked properly.

The test strip can be placed on top of or backed with glass fiber (e.g. Whatman GF/A) in order to control (i.e. speed up, or slow down the “wicking” speed) and held in place by an adhesive or other means. This brief description of the present art illustrates a completely enabled device that would allow a physician, patient, and/or technician to determine rapidly the presence or absence of anti-CMV in a patient's serum. The normal value for this CMV assay is less than 10 fmol/L of anti-CMV detected (i.e., no anti-CMV present in the serum, note that this value is purely illustrative).

To summarize Example 1 more specifically, the foregoing lateral flow/dry chemistry test strip (LFD) method for measuring the anti-CMV concentration in a serum sample using a nucleounit targeted specifically to anti-CMV comprising the steps of preparing a test means by successively impregnating a solid, absorbent, carrier matrix with liquid reagent solutions at specific locations on said test means, drying said test means, dipping completed test means into test sample or pipetting sample onto the test means, and determining the quantity of anti-CMV in said test sample by comparing the relative intensity (completeness) of the assay line produced by the reaction to the control line. It is understood that the above example was purely illustrative, and that the relative positions of the control and assay lines could be relocated without changing the spirit, scope, or intent of the instant invention.

Changes to the foregoing solutions could be made and still have similar results. The foregoing solutions could be combined together, or reduced to include only 1 solution for impregnation. The concentrations of said constituents may also be changed and still remain within the scope of the invention. The nucleounit to cocaine is produced per the manufacturing procedure for manufacturing nucleounits as previously described. It should be noted that the production of the nucleounit to CMV antibodies did not require the use of any animals and is as specific or more than an antibody or antigen to CMV antibodies. The foregoing was merely illustrative of the possibilities of this novel and unique invention.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in example 1 may be substituted with one or more selected from the group consisting of: citrate, borate, borax, sodium tetraborate decahydrate, sodium perchlorate, sodium chlorate, sodium carbonate, TRIS (Tris[hydroxymethyl]aminomethane), MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), PIPES (PiperazineN-N′-bis[2-ethanesulfonic acid)]; 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), TES (N-tris[Hydroxymethyl]methyl-2-aminomethanesulfonic acid; 2[2-Hysroxy-1,1-bis(hydroxymethyl)-ethyl]amino)ethanesulfonic acid), HEPES (N-[2-Hydroxyethyl]piperazine-N′-[2-ethanesulfonic acid]), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine; N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

The colored particles used in example 1 could be replaced with particles of any color, and made from many types of materials including rubber, latex, plastics, synthetic solids, metals, or other suitable material that will form a solid platform or substrate for the covalent attachment (binding) of a reactive compound such as a nucleounit to it.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

The material used for lateral flow in example can be selected from the group consisting of any form of absorbent, solid phase carrier that is capable of transporting a fluid to include filter paper, cellulose, synthetic resins, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

This method described serum as the matrices of choice for the analysis of CMV antibodies, however, all of the method(s) for the measurement of analytes of interest on an unknown samples using nucleounits can be performed on samples composed of biological matrices such as urine, serum, whole blood, cerebral spinal fluid, gastric fluid, sweat extracts hair homogenates, and saliva. The test sample can be made up any fluid or fluids such as beverages such water, soft drinks, beer, or mixed drinks to include liquid drinks from a bar that might be contaminated with GHB.

### EXAMPLE 2

The following procedure is a method for manufacturing a lateral flow device (LFD) for determining the concentration of cocaine and creatinine simultaneously without the aid of any other instrumentation. The measurement of creatinine concentration while simultaneously measuring cocaine will ensure the proper integrity of the specimen. If the specimen has a low creatinine (below 20 mg/dL) the urine is to dilute and the assay should be repeated with a specimen that has a normal concentration of creatinine (48 to 180 mg/dL). This lateral flow device will hold one LFD strip for cocaine and one LFD strip for creatinine in two separate channels. The lateral flow device may have the following dimensions, but can obviously be changed and still remain within the spirit and scope of the present invention. This device is approximately 100 mm long by 50 mm wide. The device is approximately 3 to 5 mm thick. The two absorbent test pads are 5 mm wide and 70 mm long. The two channels will have two holes or windows each. Each assay channel has one assay hole through which the reaction and assay results can be observed; these viewing windows are 50 mm long by 5 mm wide. Each assay channel also has two window through which to introduce sample onto the test pads. These sample holes or ports are approximately 10 mm long by 3 to 5 mm wide. The two assay channels may be on the same side of the device or one on each of the two sides. Note, the assay and sample windows are aligned with the appropriate areas of the test strips so that sample is applied to the correct location, and the appropriate reaction areas are open to view. The casing is composed of plastic, rubber, latex, wood, cardboard, or other suitable material.

The cocaine assay strip is manufactured as follows and will be placed in channel 1. The second strip for creatinine will be described in detail and will be placed into channel two.

Cocaine LFD strip is manufactured as follows:

Absorbent material is successively impregnated with the following solutions and dried at 25 degree C.:

Solution 1

0.03 M Hepes buffer pH 7.3

300 ng/mL nucleounit to colored particles

Solution 2

0.03 M Hepes buffer pH 7.3

300 ng/mL nucleounit to cocaine conjugated to red microparticles

450 ng/mL red microparticles

Solution 3

0.025 M Hepes pH 6.9

450 ng/mL cocaine

In this example, the lateral flow device is prepared in accordance with the instant invention. The lateral flow device is comprised of a paper carrier matrix impregnated with the compositions of solutions 1, 2, and 3 above. Note that said concentrations of any of the above constituents can be varied to suit the lateral flow/dipstick device format (e.g. dependent upon paper type, and inclusion of semi-permeable membranes or other innovations utilized in dry chemistry technology). Production of this test device is carried out using the following procedure. The test device made up of a solid support which includes an absorbent material capable of transporting a liquid by capillary action or wicking (e.g. nitrocellulose 5.0 u, S&S brand) in this example having dimensions of 5 mm by 70mm and can be backed by or in contact with strips of glass fiber (e.g. Whatman GF/A) to aid in controlling the wicking action. In this example, the device uses an cocaine cutoff of 300 ng/mL in urine.

The starting point or origin at which the sample is placed on the test device is 5 mm from one end of the strip, and 30 mm from this origin solution 1 (a buffered solution containing nucleounit to colored particles) is bound to the test strip 35 mm from the bottom edge of said test pad in a line approximately 1 mm wide by 5 mm long thereby extending from one side of the device to the other side forming the C, “control line”. The appearance of a colored line here after assay is complete will indicate that the lateral flow device worked properly (i.e. the sample migrated to an acceptable RF value beyond the A or assay line due to the red particles binding to the nucleounits targeted to the red particles and the binding capacity of the cocaine and the bound nucleounit to cocaine conjugated to red microparticles are reactive and nothing in the sample has adversely affected the test's reactants). A second buffered solution (solution 2) consisting of red colored particles bound (i.e. irreversibly coupled, conjugated, or covalently linked) to nucleounits to cocaine and free red particles are applied to the strip approximately 5 mm from the starting point (or 10 mm from the lower edge of the test strip) in a concentration as to make certain that assay and control lines both form solid visual lines to achieve effective results. A third buffered solution of cocaine is coupled (bound) to the strip at approximately 10 mm from the starting point of the strip (or 15 mm from the lower edge of the test strip) forming the A (assay) line approximately 1 mm wide by 5 mm long thereby extending from one side of the device to the other side forming the immobilized, coupled cocaine line. The starting point or point of origin from where the fluid (in this example serum) starts is at the 5 mm point from the end of the strip, 30 mm from the control line. This is where three drops of the sample are applied. A solid case may be used to conceal and protect all of the device except for three “windows”; one for sample application at the origin, a second at the A, assay line, and a third at the C, control line. This case may be composed of plastic, wood, cardboard, or other suitable material.

If the sample is positive, with a concentration of 300 ng/mL cocaine or more the following occurs. Three drops of urine (approximately 100 uL) are applied at the starting point or origin of the strip. The urine then migrates to the opposite or terminal end of the strip. The free cocaine present (in a concentration of 300 ng/mL or greater of cocaine) in the urine binds all of the red particles bound with the nucleounit targeted to the cocaine and these conjugated particles complexes containing cocaine bound to the nucleounit targeted to cocaine which are attached to red particles migrate with the urine toward the terminal end of the strip away from the starting point. These colored complexes will not bind to the line of cocaine bound at the 10 mm “A” line or assay window because all of the particles with nucleounits to cocaine are already bound up by the cocaine from the sample. The migrating red colored particle complexes and the free red particles continue migrating up the device until reaching the line of bound nucleounits targeted to the red particles at the “C” or control window. The cocaine-nucleounit red particle complexes and the red colored particles then bind to this line forming a solid (complete) red “control line” consisting of both types of red particles (bound and unbound).

If the sample is negative, with a concentration of less than 300 ng/mL present, the following occurs. The sample is applied and the free (unbound) nucleounit to cocaine conjugated to red microparticles complexes migrate up to the “A” assay line and bind to the cocaine conjugated to the test strip at that location thereby forming a solid (complete) red line assay line. The control particles (i.e. red particles) will keep migrating to the 35 mm “C” control line and form a solid red line to indicate the assay worked properly.

The test strip can be placed on top of or backed with glass fiber (e.g. Whatman GF/A) in order to control (i.e. speed up, or slow down the “wicking” speed) and held in place by an adhesive or other means. This brief description of the present art illustrates a completely enabled device that would allow a police officer, physician, patient, and/or technician to determine rapidly the presence or absence of cocaine in a patient's urine. The normal value for this cocaine assay is less than 300 ng/mL of cocaine detected.

To summarize Example 1 more specifically, the foregoing lateral flow/dry chemistry test strip (LFD) method for measuring the cocaine concentration in urine using a nucleounit targeted specifically to cocaine comprising the steps of preparing a test means by successively impregnating a solid, absorbent, carrier matrix with liquid reagent solutions at specific locations on said test means, drying said test means, dipping completed test means into test sample or pipetting sample onto the test means, and determining the quantity of cocaine in said test sample by comparing the relative intensity (completeness) of the assay line produced by the reaction to the control line. It is understood that the above example was purely illustrative, and that the relative positions of the control and assay lines could be relocated without changing the spirit, scope, or intent of the instant invention.

Changes to the foregoing solutions could be made and still have similar results. The foregoing solutions could be combined together, or reduced to include only 1 solution for impregnation. The concentrations of said constituents may also be changed and still remain within the scope of the invention. The nucleounit to cocaine is produced per the manufacturing procedure for manufacturing nucleounits as previously described. It should be noted that the production of the nucleounit to cocaine did not require the use of any animals and is as specific or more than an antibody to cocaine. The foregoing was merely illustrative of the possibilities of this novel and unique invention.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this section of example 2 may be substituted with one or more selected from the group consisting of: citrate, borate, phosphate, borax, sodium tetraborate decahydrate, sodium perchlorate, sodium chlorate, sodium carbonate, TRIS (Tris[hydroxymethyl]aminomethane), MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), PIPES (PiperazineN-N′-bis[2-ethanesulfonic acid)]; 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), TES (N-tris[Hydroxymethyl]methyl-2-aminomethanesulfonic acid; 2[2-Hysroxy-1,1-bis(hydroxymethyl)-ethyl]amino)ethanesulfonic acid), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine; N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

The colored particles used in example 2 could be replaced with particles of any color, and made from many types of materials including rubber, latex, plastics, synthetic solids, metals, or other suitable material that will form a solid platform or substrate for the covalent attachment (binding) of a reactive compound such as a nucleounit to it.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

The material used for lateral flow in example can be selected from the group consisting of any form of absorbent, solid phase carrier that is capable of transporting a fluid to include filter paper, cellulose, synthetic resins, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support. The flowing is a detailed description of manufacturing the second strip for creatinine after which it will be placed into channel two.

An absorbent material is successively impregnated with the following solution and dried at 25 degree C.:

Solution 1

0.05 M Tris buffer pH 7.2

150 mg/dL nucleounit to colored particles

Solution 2

0.05 M Tris buffer pH 7.2

150 mg/dL blue micro-particles

20 mg/dL nucleounit to creatinine conjugated to blue micro-particles

Solution 3

0.05 M Tris buffer pH 7.2

20 mg/dL creatinine

In this example, the lateral flow device is prepared in accordance with the instant invention. The lateral flow device is comprised of an absorbent paper carrier matrix impregnated with the composition of solutions 1, 2 and 3 from above in the appropriate locales as specified below. Note that said concentrations of any of the above constituents can be varied to suit variations employed in the specific LFD format (e.g. particular paper type, or inclusion of semi-permeable membranes or other innovations utilized in dry chemistry technology). Production of this test device is carried out using the following procedure. The test device made up of a solid support which includes an absorbent material capable of transporting a liquid by capillary action (wicking) on a piece of filter paper (e.g. nitrocellulose 5.0 u, S&S brand) in this example having dimensions of 5 mm by 70 mm and can be backed by or in contact with strips of glass fiber (e.g. Whatman GF/A) to aid in controlling the wicking action. In this example, the device uses a creatinine concentration cutoff of 20 mg/dL.

The starting point or origin at which the sample is placed on the test device is 5 mm from one end of the test pad, and 30 mm from this origin buffered solution number 1 containing nucleounit to colored particles is permanently bound to the test strip 35 mm from the bottom edge of said test pad in a line approximately 1 mm wide by 5 mm long thereby extending from one side of the device to the other side forming the C, or “control line”. The appearance of a colored line here after assay is complete will indicate that the lateral flow device worked properly (i.e. the sample migrated to an acceptable RF value beyond the A or assay line and the binding capacity of the nucleounit to creatinine and the bound creatinine are reactive and nothing in the sample has adversely affected the test's reactants). The buffered solution no. 2 consisting of blue colored particles bound (i.e. irreversibly coupled, conjugated, or covalently linked) to 20 mg/dL nucleounit to creatinine and 150 mg/dL of blue colored particles are both applied to the strip approximately 5 mm from the starting point (or 10 mm from the lower edge of the test strip) in a concentration as to make certain that assay and control lines both form solid visual lines to achieve effective results. The third buffered solution, no. 3, of 150 mg/dL creatinine is coupled (bound) to the strip at approximately 10 mm from the starting point of the strip (or 15 mm from the lower edge of the test strip) forming the A (assay) line approximately 1 mm wide by 5 mm long thereby extending from one side of the device to the other side forming the immobilized, coupled creatinine line. The test strips can be placed on top of or backed with glass fiber (e.g. Whatman GF/A) in order to control (i.e. speed up, or slow down the “wicking” speed) and held in place by an adhesive or other means. The two assay strips are then placed in the solid case to conceal and protect all of the device except for the two “windows”; one for sample application at the origin, and the second to display the A, assay line and the C, control line.

The cocaine test channel is read and evaluated as described in Example 1. The creatinine test channel is interpreted as follows. If the sample has an normal concentration of 20 mg/dL creatinine or more the following occurs. Three drops urine (approximately 100 uL) are applied at the starting point or origin of the strip. The urine then migrates to the opposite or terminal end of the strip. The free creatinine present in the urine binds all of the nucleounit to creatinine conjugated to the colored particles and these creatinine/nucleounit to creatinine/blue particle complexes also migrate with the urine toward the terminal end of the strip away from the starting point. These colored complexes will not bind to the line of creatinine bound at the 10 mm “A” line in the assay window because all of the nucleounits to creatinine on the colored particles are already bound up by the free creatinine from the sample. The migrating blue colored particle complexes, therefore, continue migrating up the device until reaching the line of bound nucleounits to colored particles at the “C” line in the assay window. The creatinine/nucleounits to creatinine/blue particle complexes then bind to this line of nucleounits to colored particles forming a solid (complete) blue “control line” consisting of creatinine/nucleounit to creatinine/blue particle/nucleounit to colored particles complexes.

If the sample is abnormal, with a concentration of less than 20 mg/dL, the following occurs. The free nucleounit to creatinine/blue particle complexes migrate up to the “A” assay line and bind to the creatinine conjugated to the test strip at that location thereby forming a solid (complete) blue line. The control particles (i.e. free colored particle complexes) will keep migrating to the 35 mm “C” control line and form a solid blue line to indicate the assay worked properly.

This brief description of the present art illustrates a completely enabled device that would allow a physician, patient, and/or technician to determine rapidly the creatinine value of the patient. The normal value for creatinine is 45 to 180 mg/dL; the utility of this assay in conjunction with the cocaine assay strip will be explained herein.

The measurement of cocaine in urine is important for overdose, pre-employment drug screening, on the job abuse of cocaine (safety) and for other reasons. If for example the urine is to dilute (not concentrated enough) the cocaine value may result in a negative due to the dilute concentration of the urine. When in fact had the urine been a normal concentration the assay would have detected the proper amount of cocaine. The attempt to adulterate urine by drinking lots of fluids prior to a drug test is well know in the art.

To summarize the creatinine strip manufacturing more specifically, the foregoing lateral flow/dry chemistry test strip (LFD) method for measuring the creatinine concentration in urine using a nucleounit targeted specifically to creatinine comprising the steps of preparing a test means by successively impregnating a solid, absorbent, carrier matrix with liquid reagent solutions at specific locations on said test means, drying said test means, dipping completed test means into test sample or pipetting sample onto the test means, and determining the quantity of creatinine in said test sample by comparing the relative intensity (completeness) of the assay line produced by the reaction to the control line. It is understood that the above example was purely illustrative, and that the relative positions of the control and assay lines could be relocated without changing the spirit, scope, or intent of the instant invention.

Changes to the foregoing solutions could be made and still have similar results. The foregoing solutions could be combined together, or reduced to include only 1 solution for impregnation. The concentrations of said constituents may also be changed and still remain within the scope of the invention. The nucleounit to creatinine is produced per the manufacturing procedure for manufacturing nucleounits as previously described. It should be noted that the production of the nucleounit to creatinine did not require the use of any animals and is as specific or more than an antibody to creatinine. The foregoing was merely illustrative of the possibilities of this novel and unique invention.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this section of example 2 may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), PIPES (PiperazineN-N′-bis[2-ethanesulfonic acid)]; 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonicacid]), TES (N-tris[Hydroxymethyl]methyl-2-aminomethanesulfonic acid; 2[2-Hysroxy-1,1-bis(hydroxymethyl)-ethyl]amino)ethanesulfonic acid), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine; N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

The colored particles used in example 2 could be replaced with particles of any color, and made from many types of materials including rubber, latex, plastics, synthetic solids, metals, or other suitable material that will form a solid platform or substrate for the covalent attachment (binding) of a reactive compound such as a nucleounit to it.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

The material used for lateral flow in example can be selected from the group consisting of any form of absorbent, solid phase carrier that is capable of transporting a fluid to include filter paper, cellulose, synthetic resins, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

### EXAMPLE 3

The following procedure is a method for manufacturing a dry chemistry lateral flow test strip (LFD) for the determination of bone loss in a test sample by measurement of its osteoporosis antigen marker concentration, in this example the targeted antigen will be Ntp. This example also illustrates the unique ability to use creatinine measurement on the same sample measured for bone loss by assaying for creatinine via liquid colorimetric methodology, dry chemistry (DCD), lateral flow (LFD), liquid antibody/antigen, liquid enzymatic assay, or other techniques to enhance the clinical significance of the bone loss (osteoporosis) assay value.

Absorbent material is successively impregnated with the following solution and dried at 25 degree C.:

Solution 1

0.05 M Pipes buffer pH 7.2

30 nM nucleounit to Ntp

Solution 2

0.05 M Pipes buffer pH 7.2

30 nM Ntp conjugated to colored micro-particles (green)

In this example the lateral flow device is prepared in accordance with the instant invention. This LFD is comprised of a paper carrier matrix impregnated in specific locations on the device with solutions 1 and 2 above. Note, the concentrations of any of the above constituents can be varied to suit the lateral flow/dipstick device format (e.g. dependent on paper type, and inclusion of semi-permeable membranes or other innovations in dry chemistry technology); the specific locations of the solutions may also be varied and still remain within the spirit and scope of this invention. The test device made up of a solid support which includes an absorbent material capable of transporting a liquid by capillary action or wicking (e.g. nitrocellulose 5.0 u, S&S brand) in this example having dimensions of 5 mm by 70 mm and can be backed by or in contact with strips of glass fiber (e.g. Whatman GF/A) to aid in controlling the wicking action. In this example, the device uses an osteoporosis cutoff of 30 nM Ntp.

Production of this test device is carried out using the following procedure. The test device made up of a solid support which includes an absorbent material capable of transporting a liquid by capillary action (wicking) on a piece of filter paper (for example, nitrocellulose 5.0 u, S&S brand) in this example having dimensions of 5 mm by 70 mm and can be backed by or in contact with strips of glass fiber (for example; Whatman GF/A) to aid in controlling the wicking action. In this example the device uses osteoporosis cutoffs of be 30 nM Ntp. The starting point or origin at which the sample is placed on the test device is 5 mm from one end of the strip, and 35 mm from this origin the buffered solution no. 1 containing nucleounit targeted to Ntp is irreversibly bound to the test strip 40 mm from the bottom edge of said test pad in a line approximately 1 mm wide by 5 mm long thereby extending from one side of the device to the other side forming the A, “assay line”. This location will indicate the concentration of target analyte present in the unknown sample tested. The second buffered solution consisting of green colored micro-particles bound (i.e. irreversibly coupled, conjugated, or covalently linked) to 30 nM Ntp is applied to the strip approximately 5 mm from the starting point (or 10 mm from the lower edge of the test strip) in a concentration as to make certain that assay line forms a solid visual line to achieve effective results. A solid case may be used to conceal and protect all of the device except for two “windows”; one for sample application at the origin, and a second at the A, assay line. This case may be composed of plastic, wood, cardboard, or other suitable material.

If the sample is positive (i.e. osteoporosis), with a concentration of 30 nM Ntp or more the following occurs. A three drops of urine (approximately 100 uL) is applied at the starting point or origin of the strip. The urine then migrates to the opposite or terminal end of the strip. The free Ntp present in the urine binds all or most active sites at the assay line at the 40 mm mark (i.e. at the “A” line; the end result is no solid colored line will form there). There is competition for the binding sites between the free Ntp and the bounds Ntp however the free Ntp is much smaller than the free anti-anti-Ntp-microparticles, and therefore migrates to the “A” line faster. A positive result is indicated by the lack of a solid line.

If the sample is negative (i.e. normal bone loss), with a concentration of less than 30 nM of Ntp, the following occurs. The free anti-anti-Ntp/green particle complexes migrate up to the “A” assay line and bind to the anti-Ntp sites conjugated to the test strip at that location thereby forming a solid (complete) green line. This occurs, because the quantity of Ntp in the sample is insufficient to bind all of the available Anti-Ntp sites on the “A” line thereby allowing sufficient numbers of the green nucleounit-Ntp-microparticles to bind there and form a solid visible line.

The test strip can be placed on top of, or backed, with glass fiber (e.g. Whatman GF/A) in order to control (i.e. speed up ,or slow down the “wicking” speed) and held in place by an adhesive or other means. This brief description of the present art illustrates a completely enabled device that would allow a physician, patient, and/or technician to determine rapidly the bone loss value of the patient. The normal value for this osteoporosis marker is 25 nM of Ntp or less; the abnormal, or elevated value is 30 nM Ntp, or greater.

To summarize Example 3 more specifically, the foregoing lateral flow/dry chemistry test strip (LFD) method to measure the osteoporosis antigen concentration for the determination of bone loss in a random urine sample, the method comprising the steps of preparing a test means by successively impregnating a solid, absorbent, carrier matrix with liquid reagent solutions at specific locations on the device, drying said test means, dipping completed test means into test sample or pipetting a known volume of urine onto the test device and determining the quantity of osteoporosis antigen in said test sample by comparing the relative intensity (completeness) of the assay line produced by the reaction to a standard chart or example. It is understood that the above example was purely illustrative, and that the relative position of the assay line could be relocated without affecting the performance of the device, or altering the scope of the invention.

Changes to the foregoing solutions could be made and still have similar results. The foregoing solutions could be combined together, or reduced to include only 1 solution for impregnation. The concentrations of said constituents may also be changed and still remain within the scope of the invention. The nucleounit to Ntp is produced per the manufacturing procedure for manufacturing nucleounits as previously described. It should be noted that the production of the nucleounit to Ntp did not require the use of any animals and is as specific or more than an antibody to Ntp. The foregoing was merely illustrative of the possibilities of this novel and unique invention. The nucleounit to Npd in the example may be replaced by any nucleounit to any of the following compounds: deoxypyridinoline (Dpd), hydroxypyridinoline (Hpd), Pyridinoloine cross-links (PCL), or N-telopeptides of type I collagen (Ntp), deoxypyridinoline cross-links (DpdC), collagen fragments with C-terminal and N-terminal ends (Cfcn), and osteocalcin (Ocn).

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

The colored particles used in example 3 could be replaced with particles of any color, and made from many types of materials including rubber, latex, plastics, synthetic solids, metals, or other suitable material that will form a solid platform or substrate for the covalent attachment (binding) of a reactive compound such as a nucleounit to it.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

The material used for lateral flow in example can be selected from the group consisting of any form of absorbent, solid phase carrier that is capable of transporting a fluid to include filter paper, cellulose, synthetic resins, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

### EXAMPLE 4

The following procedure is a method for manufacturing an aqueous, liquid reagent chemistry test for the determination of digoxin in serum on an automated chemistry analyzer or by classical, wet, manual analysis (e.g., with a spectrophotometer). Digoxin is used as cardiotonic. Its accurate measurement is of crucial importance.

Reagent Solution 1 (R1)

0.05 M Hepes buffer pH 7.2

30.0 ng/mL nucleounits targeted to Digoxin conjugated to latex microparticles

Standard 30 nM Npd Calibrator Solution

0.05 M Hepes buffer pH 7.2

3.0 ng/mL Digoxin calibrator

The reagent system of the instant invention (liquid reagent) is intended for use on any automatic chemistry analyzer with open channel capability including Olympus AU 5000 series, Dupont® Dimensions, Hitachi 700 series, Beckman CX series and others as commonly known in the art. The reagent is used in the following manner. A method for measuring for digoxin concentration comprises the steps of placing the reagent composition(s), R-1, in the reagent compartment of the chemistry autoanalyzer, aliquoting samples, calibrators, and controls into sample cups and placing them on the chemistry autoanalyzer, transferring an aliquot (e.g. 10 uL) of each sample, calibrator, and control into single discrete cuvettes mounted within the chemistry autoanalyzer, aliquoting a specified volume (e.g. 250 uL) of the reagent composition of R-1 into each cuvette and mixing, incubating the reaction mixture for a specified time interval, measuring and recording absorbance values of the reaction mixtures with the chemistry autoanalyzer's spectrophotometer at the specified wavelength (e.g. 340 n to 800 mn) at preprogrammed time intervals, and comparing absorbance values of samples and controls to those of calibrators in the form of a standard curve thereby quantitating the digoxin present. If the sample's absorbance is equal to, or greater than the 3.0 ng/mL digoxin calibrator's absorbance, this indicates elevated digoxin concentrations and consequently abnormal to toxic amounts are in the patient's serum and the sample should be diluted and rerun; if it is less than the 3.0 it is within the therapeutic range and the result can be reported out. This description of the present art illustrates a completely enabled device that would allow a physician, patient, and/or lab technician to determine the digoxin value of a patient. The normal ranges for this example are 0.8 to 2.0 ng/mL digoxin within the therapeutic range.

Changes to the foregoing solutions could be made and still have similar results. The concentrations of said constituents may be changed and still remain within the scope of the invention. Obviously, the buffers and microparticles can be altered without changing the scope of the invention.

In the instant invention, when serum is mixed with the reagent system in the prescribed ratio, the digoxin concentration will directly affect the absorbance produced by the reaction mixture. Specifically, as the free digoxin in the sample reacts with the conjugated nucleounit to digoxin-microparticle, agglutination occurs and this digoxin to nucleounit particle agglutination “colony” will absorb and/or reflect light. The comparative absorbance measurements can be made visually or via a spectrophotometer. Note, the vast majority of clinical chemistry analyzers incorporate a spectrophotometer.

Listed below is an example of parameters for the Hitachi 717 analyzer. The settings are intended as guidelines, and are set forth with the understanding that variations may be made to affect performance and still remain within the scope of the invention. Those skilled in the art will recognize that parameters may vary by instrument page 61

Specifications for the Hitachi 717 are as follows:

| Test: | [DIGOX] |
|---|---|
| say code: | [1 point] [40] − [0] |
| Sample volume: | [10] [10] |
| R1 volume | [250] [100] [NO] |
| R2 volume | [ 0] [100] [NO] |
| Wavelength | [0] [540] |
| Calib. Method: | [Linear] [0] [0] |
| Std. (1) Conc.-POS: | [ 0] − [3.0]* assigned calibrator value |
| Std. (2) Conc.-POS: | [] − [] |
| Std. (3) Conc.-POS: | [] − [] |
| Std. (4) Conc.-POS: | [] − [] |
| Std. (5) Conc.-POS: | [] − [] |
| Std. (6) Conc.-POS: | [] − [] |
| SD Limit: | [999] |
| Duplicate Limit: | [32000] |
| Sensitivity Limit: | [0] |
| ABS.Limit (INC/DEC): | [32000] [INCREASE] |
| Prozone Limit: | [250] [upper] |
| Expected Value: | [0] − [2.0] |
| Tech. Limit: | [0] − [10.0] |
| Instrument Factor | [1] |

Please note that dilution of the serum is not required before analysis. This method has a sensitivity of 0.1 ng/mL digoxin. The use of a calibrator is not necessary, if a K factor is employed. The K factor can be used in calibrating a method for analysis that utilizes enzymatic or nucleounit to antigen (in this case digoxin) reactions whose rate of change in absorbance at different concentrations forms a linear plot, and the slope of the plot is already known. The slope is based on the molar absorptivity of the absorbing species (e.g. Digoxin, or NAD . . . ) of the chemistry's reaction. The K factor can be calculated as follows; all automated instruments typically have a K factor mode.

K=total reaction volume (mL)×1000/molar absorptivity×lightpath (cm)×specimen volume (mL)

In K factor calibration, a zero or blank calibrator is run and the absorbance and concentration of this standard, and the predetermined K factor, are used in the calculation of the results of unknown samples.

The automated analysis procedure encompasses the following method for the measurement of digoxin using a nucleounit conjugated to a microparticle which is specifically targeted to digoxin in an unknown sample of serum (or other biological sample including urine, plasma, whole blood, cerebral spinal fluid, gastric fluid, hair homogenates, sweat extracts, and saliva can be used). To summarize more specifically this example, the foregoing automated method employing an aqueous liquid reagent for measuring the concentration of digoxin using nucleounits to digoxin in the reagent composition on a test specimen, said test method comprising the steps of placing the reagent composition(s) containing nucleounits to digoxin, R-1, in the reagent compartment of the chemistry autoanalyzer, aliquoting samples, calibrators, and controls into sample cups and placing them on the chemistry autoanalyzer, transferring an aliquot of each sample, calibrator, and control into single, discrete cuvettes mounted within the chemistry autoanalyzer, aliquoting a specified volume of the first reagent composition, R-1, into each cuvette and mixing, incubating the reaction mixture for a specified time interval, measuring and recording absorbance values of the reaction mixtures with the chemistry autoanalyzer's spectrophotometer at the specified monochromatic wavelength (from 340 to 800 nm) at preprogrammed time intervals, and comparing absorbance values of samples and controls to those of the calibrators in the form of a standard curve thereby quantitating the amount digoxin present. Note the wavelength can be from 340 to 800 because the analyzer is looking for an absorbance which is not specific to any particular wavelength. The use of 540 nm is this example is purely illustrative.

Changes to the foregoing solutions could be made and still have similar results. The concentrations of said constituents may also be changed and still remain within the scope of the invention. The nucleounit to digoxin is produced per the manufacturing procedure for manufacturing nucleounits as previously described. It should be noted that the production of the nucleounit to digoxin did not require the use of any animals and is as specific or more than an antibody to digoxin.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), 2-[hydroxymethyl-1,3-propanediol), ADA N-[2-Acetamidol]-2-iminodiacetic acid; N-[Carbaoylmethyl]iminodiacetc acid), ACES (2-[(2-Amino-2-oxoethyl) amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-amino ethanesulfonic acid; 2-bis(2-Hydroxyethyl) amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis [2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

The latex microparticles used in this example could be replaced with particles of any color, and made from many types of materials including rubber, plastics, synthetic solids, metals, or other suitable material that will form a solid platform or substrate for the covalent attachment (binding) of a reactive compound such as a nucleounit to it.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

### EXAMPLE 5

The following procedure is a method for manufacturing an aqueous, liquid reagent chemistry test for the determination of cannabinoids (THC) in urine on an automated chemistry analyzer or by classical, wet, manual analysis (e.g., with a spectrophotometer). Cannabinoids are considered a drug-of-abuse and a therapeutic agent. Its accurate measurement is of crucial importance.

Reagent Solution 1 (R1)

0.05 M Hepes buffer pH 7.2

300.0 ng/mL nucleounits targeted to cannabinoids conjugated to latex microparticles

Standard 30 nM Npd Calibrator Solution

0.05 M Hepes buffer pH 7.2

50.0 ng/mL cannabinoids calibrator

The reagent system of the instant invention (liquid reagent) is intended for use on any automatic chemistry analyzer with open channel capability including Olympus AU 5000 series, Dupont® Dimensions, Hitachi 700 series, Beckman CX series and others as commonly known in the art. The reagent is used in the following manner. A method for measuring for cannabinoids concentration comprises the steps of placing the reagent composition(s), R-1, in the reagent compartment of the chemistry autoanalyzer, aliquoting samples, calibrators, and controls into sample cups and placing them on the chemistry autoanalyzer, transferring an aliquot (e.g. 10 uL) of each sample, calibrator, and control into single discrete cuvettes mounted within the chemistry autoanalyzer, aliquoting a specified volume (e.g. 250 uL) of the reagent composition of R-1 into each cuvette and mixing, incubating the reaction mixture for a specified time interval, measuring and recording absorbance values of the reaction mixtures with the chemistry autoanalyzer's spectrophotometer at the specified wavelength (e.g. 340 nm to 800 nm) at preprogrammed time intervals, and comparing absorbance values of samples and controls to those of calibrators in the form of a standard curve thereby quantitating the cannabinoids present. If the sample's absorbance is equal to, or greater than the 50.0 ng/mL cannabinoids calibrator's absorbance, this indicates a positive result for the presence of cannabinoids in urine and consequently abnormal. The analytical range of this assay is 300 ng/mL cannabinoids and therefore a result of 50.0 or greater would be within the range and can be reported out. This description of the present art illustrates a completely enabled device that would allow an enforcement official, physician, patient, and/or lab technician to determine the cannabinoids value of an individual. The normal ranges for this example are 0.0 to less than 50.0 ng/mL cannabinoids.

Changes to the foregoing solutions could be made and still have similar results. The concentrations of said constituents may be changed and still remain within the scope of the invention. Obviously, the buffers and microparticles can be altered without changing the scope of the invention.

In the instant invention, when urine is mixed with the reagent system in the prescribed ratio, the cannabinoids concentration will directly affect the absorbance produced by the reaction mixture. Specifically, as the free cannabinoids in the sample reacts with the conjugated nucleounit to cannabinoids-microparticle, agglutination occurs and this cannabinoids to nucleounit particle agglutination “colony” will absorb and/or reflect light. The comparative absorbance measurements can be made visually or via a spectrophotometer. Note, the vast majority of clinical chemistry analyzers incorporate a spectrophotometer.

Listed below is an example of parameters for the Hitachi 717 analyzer. The settings are intended as guidelines, and are set forth with the understanding that variations may be made to affect performance and still remain within the scope of the invention. Those skilled in the art will recognize that parameters may vary by instrument. page 61

Specifications for the Hitachi 717 are as follows:

| Test: | [THC] |
|---|---|
| say code: | [1 point] [40] − [0] |
| Sample volume: | [10] [10] |
| R1 volume | [250] [100] [NO] |
| R2 volume | [ 0] [100] [NO] |
| Wavelength | [0] [340] |
| Calib. Method: | [Linear] [0] [0] |
| Std. (1) Conc.-POS: | [ 0] − [50.0]* assigned calibrator value |
| Std. (2) Conc.-POS: | [] − [] |
| Std. (3) Conc.-POS: | [] − [] |
| Std. (4) Conc.-POS: | [] − [] |
| Std. (5) Conc.-POS: | [] − [] |
| Std. (6) Conc.-POS: | [] − [] |
| SD Limit: | [999] |
| Duplicate Limit: | [32000] |
| Sensitivity Limit: | [0] |
| ABS. Limit (INC/DEC): | [32000] [INCREASE] |
| Prozone Limit: | [250] [upper] |
| Expected Value: | [0] − [50.0] |
| Tech. Limit: | [0] − [300.0] |
| Instrument Factor | [1] |

Please note that dilution of the urine is not required before analysis. This method has a sensitivity of 1.0 ng/mL cannabinoids. The use of a calibrator is not necessary, if a K factor is employed. The K factor can be used in calibrating a method for analysis that utilizes enzymatic or nucleounit to antigen (in this case cannabinoids) reactions whose rate of change in absorbance at different concentrations forms a linear plot, and the slope of the plot is already known. The slope is based on the molar absorptivity of the absorbing species (e.g. cannabinoids, Digoxin, or NAD . . . ) of the chemistry's reaction. The K factor can be calculated as follows; all automated instruments typically have a K factor mode.

K=total reaction volume (mL)×1000/molar absorptivity×lightpath (cm)×specimen volume (mL)

In K factor calibration, a zero or blank calibrator is run and the absorbance and concentration of this standard, and the predetermined K factor, are used in the calculation of the results of unknown samples.

The automated analysis procedure encompasses the following method for the measurement of cannabinoids using a nucleounit conjugated to a microparticle which is specifically targeted to cannabinoids in an unknown sample of urine (or other biological sample including serum, plasma, whole blood, cerebral spinal fluid, gastric fluid, hair homogenates, sweat extracts, and saliva can be used). To summarize more specifically this example, the foregoing automated method employing an aqueous liquid reagent for measuring the concentration of cannabinoids using nucleounits on a test specimen, said test method comprising the steps of placing the reagent composition(s) containing nucleounits to cannabinoids, R-1, in the reagent compartment of the chemistry autoanalyzer, aliquoting samples, calibrators, and controls into sample cups and placing them on the chemistry autoanalyzer, transferring an aliquot of each sample, calibrator, and control into single, discrete cuvettes mounted within the chemistry autoanalyzer, aliquoting a specified volume of the first reagent composition, R-1, into each cuvette and mixing, incubating the reaction mixture for a specified time interval, measuring and recording absorbance values of the reaction mixtures with the chemistry autoanalyzer's spectrophotometer at the specified monochromatic wavelength (from 340 to 800 nm) at preprogrammed time intervals, and comparing absorbance values of samples and controls to those of the calibrators in the form of a standard curve thereby quantitating the amount digoxin present. Note the wavelength can be from 340 to 800 because the analyzer is looking for an absorbance which is not specific to any particular wavelength. The use of 340 nm is this example is purely illustrative.

Changes to the foregoing solutions could be made and still have similar results. The concentrations of said constituents may also be changed and still remain within the scope of the invention. The nucleounit to cannabinoids is produced per the manufacturing procedure for manufacturing nucleounits as previously described. It should be noted that the production of the nucleounit to cannabinoids did not require the use of any animals and is as specific or more than an antibody to cannabinoids.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

The latex microparticles used in this example could be replaced with particles of any color, and made from many types of materials including rubber, plastics, synthetic solids, metals, or other suitable material that will form a solid platform or substrate for the covalent attachment (binding) of a reactive compound such as a nucleounit to it.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

### EXAMPLE 6

The following procedure is a method for manufacturing a dry chemistry test strip (DCD), for the determination of phencyclidine (PCP) in a test sample. It is known in the field of drugs-of-abuse testing that PCP is a drug of abuse and has little clinical value. Therefore, the presence of PCP is indicative of drug abuse.

Filter paper is impregnated with the following solutions and dried at 25 degree C.:

Solution 1

0.05 M TES buffer

0.05 Units/mL beta-Galactosidase/nucleounit targeted to PCP (enzyme conjugated to nucleounit for PCP)

add to 900 mL D.I. water, mix, adjust pH to 7.1, Q.S. to 1000 mL

Solution 2

0.01 M N-Methylindoxyl-beta-D-galactopyranoside

1 mL (0.1%) DMSO

dissolve in 900.0 mL distilled water, mix, and Q.S. to 1000 mL.

In this example, a dipstick is prepared in accordance with the instant invention and was prepared in accordance with the instant invention. The DCD device is comprised of a paper carrier matrix impregnated with the composition of solution. Note, the concentrations of any of the above constituents can be varied to suit the device format (e.g. dependent upon paper type, and inclusion of semi-permeable membranes or other innovations utilized in dry chemistry technology).

Production of this test device is carried out using the following procedure. The test device, a piece of Whatman 3 MM filter paper having dimensions of 0.25 inch by 3 inches is impregnated with solution 1 by immersion into it. The paper is then dried by using forced air not exceeding 60 degrees C. After drying the paper is then impregnated with solution 2 by immersion and dried by forced air not to exceed 60 degrees C. The paper is then cut into smaller pieces measuring 0.25 inches by 0.25 inches. The paper is then laminated to one side of a double-sided adhesive transfer tape commercially available from 3M Company, St. Paul, Minn. 55144. This laminate (paper plus adhesive) measures 0.25 inches by 0.25 inches. The laminate is then attached, via the unused adhesive side, to one end of a sturdy polystyrene strip measuring about 0.25 inches by 3 inches; the resulting product forms a test device comprising a 3 inch long polystyrene handle with a square of the impregnated test paper at one end. The dipstick thus obtained will produce a greenish to blue/purple color when exposed to PCP at a concentration of 25 ng/mL PCP or greater. In fact, the intensity of the color is proportional to the concentration of PCP present in the sample. The test device, therefore, effectively measures the presence and concentration of PCP in the urine.

To summarize Example 6 more specifically, the foregoing dry chemistry test strip (DCD) method to measure PCP concentration in a urine sample, the method comprising the steps of preparing a test means by successively impregnating a carrier matrix with reagent solutions containing nucleounits targeted to PCP, drying said test means, dipping completed test means into a test sample, and determining the quantity of PCP present in said test sample by comparing the relative intensity and color produced by the reaction to a color chart with color blocks referenced to specific concentrations of PCP.

As described in Example 6, however, an additional solution is required. This solution 2 is incorporated into the test device by immersing the test paper into solution 2; the paper is then dried by using forced air not exceeding 60 degrees C. as previously described. A two-part test pad “sandwich” could be used and in that case the pad with solution #1 must be on top and the pad with solution 2 is on the bottom. The dipstick thus obtained will produce a greenish to blue/purple color when exposed to PCP at a concentration of 25 ng/mL or greater. In fact, the intensity of the color is proportional to the concentration of PCP in the sample. This test device, therefore, effectively identifies PCP in urine or other biological or liquid matrices using a nucleounit manufactured to be specifically targeted to PCP.

Changes to the foregoing solutions could be made and still have similar results. The foregoing solutions could be combined together, or reduced to include only 1. The concentrations of said constituents may also be changed and still remain within the scope of the invention.

The indicator substrate complex in the solutions 1 and 2, could be substituted with one or more of the following: 4-Aminophenyl-beta-D-galactopyranoside, 3-indoxyl-beta-D-galactopyranoside (blue), 5-Bromo-4-chloro-3-indoxyl-beta-D-galactopyranoside (blue), 5-Bromo-3-indoxyl-beta-D-galactopyranoside (blue), 6-chloro-3-indoxyl-beta-D-galactopyranoside (salmon), 5-bromo-6-chloro-3-indoxyl-beta-D-galactopyranoside (Magenta-beta-D-Gal) 6-Fluoro-3-indoxyl-beta-D-galactopyranoside, 8-Hydroxyquinoline-beta-D-galactopyranoside, 5-Iodo-3-indoxyl-beta-D-galactopyranoside (purple), 2-Nitrophenyl-beta-D-galactopyranoside, 4-Nitrophenyl-beta-D-galactopyranoside, Naphthol AS-BI-beta-D-galactopyranoside, and 2-Naphthyl-beta-D-galactopyranoside (yellow). Fluorescent substrates may also be utilized including 4-Methylumbelliferyl-beta-D-glucuronic acid. The colors noted in the parentheses are those produced in the reaction described above. The indicator substrate used in these examples must be matched to the conformation of the galactosidase used (i.e. alpha or beta, and dextrarotorary (D) or levorotorary (L)). For example, beta-D-Galactosidase should be matched with the indicator/substrate Iodo-3-indoxyl-beta-D-galactopyranoside; conversely, alpha-L-Galactosidase would be matched with Iodo-3-indoxyl-alpha-L-galactopyranoside. Note that some cross-reactivity does occur between stereo-isomers and, therefore, it is possible to substitute these compounds where appropriate.

Substitution of the beta-Galactosidase with another enzyme would necessitate a change of substrate indicator complex. If another glycosidase was selected, it would have to be matched to the appropriate substrate (e.g. beta-Cellobiosidase and a cellobioside). Examples of substrates for beta-D-Cellobiosidase include 5-Bromo-4-chloro-3-indoxyl-beta-D-cellobioside, 5-Bromo-6-chloro-3-indoxyl-beta-D-cellobioside, 4-Nitrophenyl-beta-D-cellobioside, 1-Naphthyl-cellobioside, and the fluorescent indicator, 4-Methylumbelliferyl-beta-D-cellobioside.

Other glycosidases which may be substituted for Galactosidase and Cellobiosidase include the alpha and beta, and D and L conformations of the following enzymes: Arabinosidase, Fucosidase, Galactosaminidase, Glucosaminidase, Glucosidase, Glucuronidase, Lactosidase, Maltosidase, Mannosidase, and Xylosidase. Their corresponding substrates, Arabinopyranoside, Fucopyranoside, Galactosaminide, Glucosaminide, Glucopyranoside, Glucuronic acid, Lactopyranoside, Maltopyranoside, Mannopyranoside, and Xylopyranoside may be bound to each of the following color indicator groups: 5-Bromo-4-chloro-3-indoxyl, 5-Bromo-6-chloro-3-indoxyl, 6-chloro-3-indoxyl, 5-Bromo-3-indoxyl, 5-Iodo-3-indoxyl, 3-indoxyl, 2-(6-Bromonaphthyl), 6-Fluoro-3-indoxyl 2-Nitrophenyl, 4-Nitrophenyl, 1-Naphthyl, Naphthyl AS-BI, 2-Nitrophenyl-N-acetyl, 4-Nitrophenyl-N-acetyl, and 4-Methylumbelliferyl moieties.

The glycosidase enzyme conjugated to PCP in the example above can also be replaced by other types of enzymes whose substrates are compatible with the indicator groups listed above. These include esterases (e.g. Carboxyl esterase, and Cholesterol esterase), sulfatases (e.g. Aryl sufatase), and phosphatases (e.g. Alkaline phosphatase). These enzymes can utilize the indicator groups delineated above when conjugated to the corresponding substrate. For example, Carboxyl esterase and 6-chloro-3-indoxyl butyrate, and Aryl sulfatase and 5-bromo-4-chloro-3-indoxyl sulfate, and alkaline phosphatase and 2-naphthyl phosphate form enzyme-substrate pairs.

Other enzymes may be conjugated to PCP, and therefore substituted for the species described above. This group now listed, however, must utilize a substrate that is distinct and separate from the indicator. This enzyme group may include any dehydrogenase, oxidase, hydroxylase, or oxidoreductase. Each grouping will utilize a specific indicator or group of indicators. The dehydrogenases and hydroxylases will utilize a co-enzyme, a color indicator and an electron carrier such as a-NAD (a-Nicotinamide adenine dinucleotide), however this electron carrier/acceptor can be replaced by the alpha or beta isomers of any one of the following substitutes: nicotinamide adenine dinucleotide, nicotinamide adenine dinucleotide 3′-phosphate, nicotinamide adenine dinucleotide phosphate, triphosphopyridine, nicotinamide 1-N1-ethenoadenine dinucleotide phosphate, nicotinamide hypoxanthine dinucleotide, nicotinamide hypoxanthine dinucleotide phosphate, nicotinamide mononucleotide, nicotinamide N1-propylsulfonate, nicotinamide ribose monophosphate, or other analogs of NAD.

Some dehydrogenases and hydroxylases and their substrate pairs which can be used include Formaldehyde dehydrogenase and Formaldehyde, Fructose dehydrogenase and Fructose, Glucose-6-phosphate dehydrogenase and Glucose-6-phosphate, Glucose dehydrogenase and Glucose, Glutamate dehydrogenase and Glutamate, Glycerol dehydrogenase and Glycerol, Glycerol-3-phosphate dehydrogenase and Glycerol-3-phosphate, Hydroxybutyrate dehydrogenase and Hydroxybutyrate, Hydroxybenzoate hydroxylase and 4-Hydroxybenzoate, Lactate dehydrogenase and Lactate, Leucine dehydrogenase and Leucine, Malate dehydrogenase and Malate, Mannitol dehydrogenase and Mannitol, or any other dehydrogenase or hydroxylase.

The use of oxidases to replace the glycosidase also requires a separate indicator, and peroxidase. Some oxidases and their substrate pair which can be used include Acyl-CoA oxidase and Acyl-CoA, Alcohol oxidase and Ethanol, Ascorbate oxidase and Ascorbate, Cholesterol oxidase and Cholesterol, Choline oxidase and Choline, Glucose oxidase and Glucose, Glycerophosphate oxidase and Glycerophosphate, Xanthine oxidase and Xanthine, Uricase and Uric acid, or any other oxidase.

A few color indicators that can be utilized with peroxidase include pyrogallol, ABTS (2,2′-Azinobis(3-ethylbenzthiazoline) sulfonic acid), 3,3′,5,5′-Tetramethylbenzidine, ortho-Dianisidine, 3,3′-Diaminibenzidine, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), and N,N-Bis(4-sulfobutyl)-3,5-dimethylaniline (MADB). An indicator pair may also be used. One such pair is 3-Methyl-2-benzothiazolinonehydrazone and Dimerhylaniline. Another pair combines 4-aminoantipyrine with a number of compounds to create a violet to violet-blue color complex in the presence of the peroxide/peroxidase reaction. These compounds include phenol, 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, and N-Ethyl-N-(2-hydroxy-3-Sulfopropyl)-m-toluidine. An example of this assay procedure would substitute glucose oxidase for galactosidase in the nucleounit to PCP-enzyme conjugate in R-1; the R-2 would then contain glucose as the substrate and ABTS (reduced) as the indicator. The R-2 would also contain peroxidase, because the product of the reaction between glucose oxidase and glucose yields peroxide. The peroxidase oxidizes any peroxide thus produced, thereby releasing an oxygen atom; this oxygen, in turn, reacts with ABTS, and converts it from the colorless, reduced form to its blue, oxidized form. The intensity of the blue color produced is proportional to the PCP concentration present in the specimen. Clearly, peroxidase may be conjugated to the nucleounit that is targeted to PCP or any other analyte of interest, and the indicators noted above used with it and its substrate, peroxide.

The use of oxireductases to replace glycosidase also requires a separate indicator including NADPH oxidoreductase and NADPH, or any oxidoreductase. The NADPH oxireductase reduces the NADPH in the presence of Flavin mononucleotide (FMN). This reaction may be observed visually by utilizing the same color indicators as delineated for the dehydrogenases, or measured spectrophotometrically at 340 nm.

Changes to the foregoing solutions could be made and still have similar results. The concentrations of said constituents may also be changed and still remain within the scope of the invention. The nucleounit to PCP is produced per the manufacturing procedure for manufacturing nucleounits as previously described. It should be noted that the production of the nucleounit to PCP did not require the use of any animals and is as specific or more than an antibody to PCP.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

Manufacture of the dry chemistry dipsticks may require the addition of thickeners as taught in the art. Some compounds commonly used for this purpose include: polyvinylpyrrolidone, algin, carragenin, casein, albumin, methyl cellulose, and gelatin. The typical range of concentration for these thickeners is about 0.5 to 5.0 g. per 100 ml. Wetting agents or surfactants are also typically used in dry chemistry. For dry chemistry applications, wetting agents aid in even distribution of the chemicals and promote even color development. Acceptable wetting agents can be hydrophilic polymers, or cationic, anionic, amphoteric, or nonionic species. Some commonly used wetting agents include sodium dodecyl-benzene sulphonate, sodium lauryl sulphate, benzalkonium chloride, N-lauroylsarcosine sodium salt, Brij-35, Tween 20, Triton X-100, dioctyl sodium sulphosuccinate, and polyethylene glycol 6000. Wetting agents can be added to dipstick impregnation solutions in amounts of 0.5% to 5.0%, and 0.1% to 1.0% in liquid reagents.

Color enhancers may be used such as sucrose, lactose, glucose or other compounds. Color enhancement can be defined as intensification and/or alteration in some manner the color that is produced by the reaction to improve the measurement of the detectable response.

The production of dry chemistry test strips for the present invention can utilize any form of absorbent, solid phase carrier including filter paper, cellulose or synthetic resin fleeces in conjunction with liquid solutions of reagent compositions in volatile solvents. This can be carried out in one or more impregnation steps. Each impregnation may contain one or more of the chemical compounds making up the assay reagent composition; the exact procedure is dictated by the inter-reactivity of the assay constituents and the order in which they may have to react with the analyte of interest.

In the case of the DLFH or LFD of the present invention it can utilize any form of absorbent, solid phase carrier that is capable of transporting a fluid. These can include filter paper, cellulose or synthetic resins. More specifically, the lateral flow material can include cellulose, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

After impregnation, the dipsticks are dried, cut into strips, glued to a support structure (usually a flexible, flat, plastic stick made up of polystyrene, vinyl polypropylene, and polyester or other suitable support material) as part of a “sandwich” composed of the handle, test pad, and a synthetic resin film and/or a fine-mesh material in the manner described in German Pat. No, 2,118,455. In addition, the instant invention may be combined with the water-stable film as taught in U.S. Pat. No. 3,530,957 to produce a dipstick in which the excess sample fluid can be wiped off in order to improve the accuracy and precision of the results.

### EXAMPLE 7

The following procedure is a method for manufacturing a dry chemistry test strip (DCD), for the determination of cholesterol in a serum test sample. It is known in the field of clinical diagnostics that the measurement of cholesterol levels is very important in determination of health of the individual.

Filter paper is impregnated with the following solutions and dried at 25 degree C.:

Solution 1

0.05 M DIPSO (3-[N,N-bis(2-Hydroxyethyl)amino]-2-hydroxypropanesulfonic acid) buffer

0.05 Units/mL beta-Galactosidase/nucleounits targeted to PCP (enzyme conjugated to nucleounit for PCP)

add to 900 mL D.I. water, mix, adjust pH to 7.1, Q.S. to 1000 mL

Solution 2

0.01 M 8-Hydroxyquinoline-beta-D-galactopyrano-side

1 mL (0.1%) DMSO

dissolve in 900.0 mL distilled water, mix, and Q.S. to 1000 mL.

In this example, a dipstick is prepared in accordance with the instant invention and was prepared in accordance with the instant invention. The DCD device is comprised of a paper carrier matrix impregnated with the composition of solution. Note, the concentrations of any of the above constituents can be varied to suit the device format (e.g. dependent upon paper type, and inclusion of semi-permeable membranes or other innovations utilized in dry chemistry technology).

Production of this test device is carried out using the following procedure. The test device, a piece of Whatman 3 MM filter paper having dimensions of 0.25 inch by 3 inches is impregnated with solution 1 by immersion into it. The paper is then dried by using forced air not exceeding 60 degrees C. After drying the paper is then impregnated with solution 2 by immersion and dried by forced air not to exceed 60 degrees C. The paper is then cut into smaller pieces measuring 0.25 inches by 0.25 inches. The paper is then laminated to one side of a double-sided adhesive transfer tape commercially available from 3M Company, St. Paul, Minn. 55144. This laminate (paper plus adhesive) measures 0.25 inches by 0.25 inches. The laminate is then attached, via the unused adhesive side, to one end of a sturdy polystyrene strip measuring about 0.25 inches by 3 inches; the resulting product forms a test device comprising a 3 inch long polystyrene handle with a square of the impregnated test paper at one end. The dipstick thus obtained will produce a greenish to blue/purple color when exposed to Cholesterol at a concentration of equal to or greater than 300 mg/dL. In fact, the intensity of the color is proportional to the concentration of cholesterol present in the sample. The test device, therefore, effectively measures the presence and concentration of Cholesterol in serum.

To summarize Example 6 more specifically, the foregoing dry chemistry test strip (DCD) method to measure cholesterol concentration in a serum sample, the method comprising the steps of preparing a test means by successively impregnating a carrier matrix with reagent solutions containing nucleounits targeted to cholesterol, drying said test means, dipping completed test means into a test sample, and determining the quantity of cholesterol present in said test sample by comparing the relative intensity and color produced by the reaction to a color chart with color blocks referenced to specific concentrations of cholesterol.

As described in Example 6, however, an additional solution is required. This solution 2 is incorporated into the test device by immersing the test paper into solution 2; the paper is then dried by using forced air not exceeding 60 degrees C. as previously described. A two-part test pad “sandwich” could be used and in that case the pad with solution #1 must be on top and the pad with solution 2 is on the bottom. The dipstick thus obtained will produce a bluish to blue/purple color when exposed to cholesterol at a concentration of 300 mg/dL or greater. In fact, the intensity of the color is proportional to the concentration of cholesterol in the sample. This test device, therefore, effectively identifies cholesterol in serum or other biological matrices using a nucleounit manufactured to be specifically targeted to cholesterol.

Changes to the foregoing solutions could be made and still have similar results. The foregoing solutions could be combined together, or reduced to include only 1. The concentrations of said constituents may also be changed and still remain within the scope of the invention.

The indicator substrate complex in the solutions 1 and 2, could be substituted with one or more of the following: 4-Aminophenyl-beta-D-galactopyranoside, 3-indoxyl-beta-D-galactopyranoside (blue), 5-Bromo-4-chloro-3-indoxyl-beta-D-galactopyranoside (blue), 5-Bromo-3-indoxyl-beta-D-galactopyranoside (blue), 6-chloro-3-indoxyl-beta-D-galactopyranoside (salmon), 5-bromo-6-chloro-3-indoxyl-beta-D-galactopyranoside (Magenta-beta-D-Gal) 6-Fluoro-3-indoxyl-beta-D-galactopyranoside, N-Methylindoxyl-beta-D-galactopyranoside, 5-Iodo-3-indoxyl-beta-D-galactopyranoside (purple), 2-Nitrophenyl-beta-D-galactopyranoside, 4-Nitrophenyl-beta-D-galactopyranoside, Naphthol AS-BI-beta-D-galactopyranoside, and 2-Naphthyl-beta-D-galactopyranoside (yellow). Fluorescent substrates may also be utilized including 4-Methylumbelliferyl-beta-D-glucuronic acid. The colors noted in the parentheses are those produced in the reaction described above. The indicator substrate used in these examples must be matched to the conformation of the galactosidase used (i.e. alpha or beta, and dextrarotorary (D) or levorotorary (L)). For example, beta-D-Galactosidase should be matched with the indicator/substrate Iodo-3-indoxyl-beta-D-galactopyranoside; conversely, alpha-L-Galactosidase would be matched with Iodo-3-indoxyl-alpha-L-galactopyranoside. Note that some cross-reactivity does occur between stereo-isomers and, therefore, it is possible to substitute these compounds where appropriate.

Substitution of the beta-Galactosidase with another enzyme would necessitate a change of substrate indicator complex. If another glycosidase was selected, it would have to be matched to the appropriate substrate (e.g. beta-Cellobiosidase and a cellobioside). Examples of substrates for beta-D-Cellobiosidase include 5-Bromo-4-chloro-3-indoxyl-beta-D-cellobioside, 5-Bromo-6-chloro-3-indoxyl-beta-D-cellobioside, 4-Nitrophenyl-beta-D-cellobioside, 1-Naphthyl-cellobioside, and the fluorescent indicator, 4-Methylumbelliferyl-beta-D-cellobioside.

Other glycosidases which may be substituted for Galactosidase and Cellobiosidase include the alpha and beta, and D and L conformations of the following enzymes: Arabinosidase, Fucosidase, Galactosaminidase, Glucosaminidase, Glucosidase, Glucuronidase, Lactosidase, Maltosidase, Mannosidase, and Xylosidase. Their corresponding substrates, Arabinopyranoside, Fucopyranoside, Galactosaminide, Glucosaminide, Glucopyranoside, Glucuronic acid, Lactopyranoside, Maltopyranoside, Mannopyranoside, and Xylopyranoside may be bound to each of the following color indicator groups: 5-Bromo-4-chloro-3-indoxyl, 5-Bromo-6-chloro-3-indoxyl, 6-chloro-3-indoxyl, 5-Bromo-3-indoxyl, 5-Iodo-3-indoxyl, 3-indoxyl, 2-(6-Bromonaphthyl), 6-Fluoro-3-indoxyl 2-Nitrophenyl, 4-Nitrophenyl, 1-Naphthyl, Naphthyl AS-BI, 2-Nitrophenyl-N-acetyl, 4-Nitrophenyl-N-acetyl, and 4-Methylumbelliferyl moieties.

The glycosidase enzyme conjugated to cholesterol in the example above can also be replaced by other types of enzymes whose substrates are compatible with the indicator groups listed above. These include esterases (e.g. Carboxyl esterase, and Cholesterol esterase), sulfatases (e.g. Aryl sufatase), and phosphatases (e.g. Alkaline phosphatase). These enzymes can utilize the indicator groups delineated above when conjugated to the corresponding substrate. For example, Carboxyl esterase and 6-chloro-3-indoxyl butyrate, and Aryl sulfatase and 5-bromo-4-chloro-3-indoxyl sulfate, and alkaline phosphatase and 2-naphthyl phosphate form enzyme-substrate pairs.

Other enzymes may be conjugated to cholesterol, and therefore substituted for the species described above. This group now listed, however, must utilize a substrate that is distinct and separate from the indicator. This enzyme group may include any dehydrogenase, oxidase, hydroxylase, or oxidoreductase. Each grouping will utilize a specific indicator or group of indicators. The dehydrogenases and hydroxylases will utilize a co-enzyme, a color indicator and an electron carrier such as a-NAD (a-Nicotinamide adenine dinucleotide), however this electron carrier/acceptor can be replaced by the alpha or beta isomers of any one of the following substitutes: nicotinamide adenine dinucleotide, nicotinamide adenine dinucleotide 3′-phosphate, nicotinamide adenine dinucleotide phosphate, triphosphopyridine, nicotinamide 1-N1-ethenoadenine dinucleotide phosphate, nicotinamide hypoxanthine dinucleotide, nicotinamide hypoxanthine dinucleotide phosphate, nicotinamide mononucleotide, nicotinamide N1-propylsulfonate, nicotinamide ribose monophosphate, or other analogs of NAD.

Some dehydrogenases and hydroxylases and their substrate pairs which can be used include Formaldehyde dehydrogenase and Formaldehyde, Fructose dehydrogenase and Fructose, Glucose-6-phosphate dehydrogenase and Glucose-6-phosphate, Glucose dehydrogenase and Glucose, Glutamate dehydrogenase and Glutamate, Glycerol dehydrogenase and Glycerol, Glycerol-3-phosphate dehydrogenase and Glycerol-3-phosphate, Hydroxybutyrate dehydrogenase and Hydroxybutyrate, Hydroxybenzoate hydroxylase and 4-Hydroxybenzoate, Lactate dehydrogenase and Lactate, Leucine dehydrogenase and Leucine, Malate dehydrogenase and Malate, Mannitol dehydrogenase and Mannitol, or any other dehydrogenase or hydroxylase.

The use of oxidases to replace the glycosidase also requires a separate indicator, and peroxidase. Some oxidases and their substrate pair which can be used include Acyl-CoA oxidase and Acyl-CoA, Alcohol oxidase and Ethanol, Ascorbate oxidase and Ascorbate, Cholesterol oxidase and Cholesterol, Choline oxidase and Choline, Glucose oxidase and Glucose, Glycerophosphate oxidase and Glycerophosphate, Xanthine oxidase and Xanthine, Uricase and Uric acid, or any other oxidase.

A few color indicators that can be utilized with peroxidase include pyrogallol, ABTS (2,2′-Azinobis(3-ethylbenzthiazoline) sulfonic acid), 3,3′,5,5′-Tetramethylbenzidine, ortho-Dianisidine, 3,3′-Diaminibenzidine, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), and N,N-Bis(4-sulfobutyl)-3,5-dimethylaniline (MADB). An indicator pair may also be used. One such pair is 3-Methyl-2-benzothiazolinonehydrazone and Dimerhylaniline. Another pair combines 4-aminoantipyrine with a number of compounds to create a violet to violet-blue color complex in the presence of the peroxide/peroxidase reaction. These compounds include phenol, 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, and N-Ethyl-N-(2-hydroxy-3-Sulfopropyl)-m-toluidine. An example of this assay procedure would substitute glucose oxidase for galactosidase in the nucleounit to PCP-enzyme conjugate in R-1; the R-2 would then contain glucose as the substrate and ABTS (reduced) as the indicator. The R-2 would also contain peroxidase, because the product of the reaction between glucose oxidase and glucose yields peroxide. The peroxidase oxidizes any peroxide thus produced, thereby releasing an oxygen atom; this oxygen, in turn, reacts with ABTS, and converts it from the colorless, reduced form to its blue, oxidized form. The intensity of the blue color produced is proportional to the cholesterol concentration present in the specimen. Clearly, peroxidase may be conjugated to the nucleounit that is targeted to cholesterol or any other analyte of interest, and the indicators noted above used with it and its substrate, peroxide.

The use of oxireductases to replace glycosidase also requires a separate indicator including NADPH oxidoreductase and NADPH, or any oxidoreductase. The NADPH oxireductase reduces the NADPH in the presence of Flavin mononucleotide (FMN). This reaction may be observed visually by utilizing the same color indicators as delineated for the dehydrogenases, or measured spectrophotometrically at 340 nm.

Changes to the foregoing solutions could be made and still have similar results. The concentrations of said constituents may also be changed and still remain within the scope of the invention. The nucleounit to cholesterol is produced per the manufacturing procedure for manufacturing nucleounits as previously described. It should be noted that the production of the nucleounit to cholesterol did not require the use of any animals and is as specific or more than an antibody to cholesterol.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

Manufacture of the dry chemistry dipsticks may require the addition of thickeners as taught in the art. Some compounds commonly used for this purpose include: polyvinylpyrrolidone, algin, carragenin, casein, albumin, methyl cellulose, and gelatin. The typical range of concentration for these thickeners is about 0.5 to 5.0 g. per 100 ml. Wetting agents or surfactants are also typically used in dry chemistry. For dry chemistry applications, wetting agents aid in even distribution of the chemicals and promote even color development. Acceptable wetting agents can be hydrophilic polymers, or cationic, anionic, amphoteric, or nonionic species. Some commonly used wetting agents include sodium dodecyl-benzene sulphonate, sodium lauryl sulphate, benzalkonium chloride, N-lauroylsarcosine sodium salt, Brij-35, Tween 20, Triton X-100, dioctyl sodium sulphosuccinate, and polyethylene glycol 6000. Wetting agents can be added to dipstick impregnation solutions in amounts of 0.5% to 5.0%, and 0.1% to 1.0% in liquid reagents.

Color enhancers may be used such as sucrose, lactose, glucose or other compounds. Color enhancement can be defined as intensification and/or alteration in some manner the color that is produced by the reaction to improve the measurement of the detectable response.

The production of dry chemistry test strips for the present invention can utilize any form of absorbent, solid phase carrier including filter paper, cellulose or synthetic resin fleeces in conjunction with liquid solutions of reagent compositions in volatile solvents. This can be carried out in one or more impregnation steps. Each impregnation may contain one or more of the chemical compounds making up the assay reagent composition; the exact procedure is dictated by the inter-reactivity of the assay constituents and the order in which they may have to react with the analyte of interest.

In the case of the DLFH or LFD of the present invention it can utilize any form of absorbent, solid phase carrier that is capable of transporting a fluid. These can include filter paper, cellulose or synthetic resins. More specifically, the lateral flow material can include cellulose, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

After impregnation, the dipsticks are dried, cut into strips, glued to a support structure (usually a flexible, flat, plastic stick made up of polystyrene, vinyl polypropylene, and polyester or other suitable support material) as part of a “sandwich” composed of the handle, test pad, and a synthetic resin film and/or a fine-mesh material in the manner described in German Pat. No, 2,118,455. In addition, the instant invention may be combined with the water-stable film as taught in U.S. Pat. No. 3,530,957 to produce a dipstick in which the excess sample fluid can be wiped off in order to improve the accuracy and precision of the results.

### EXAMPLE 8

The following procedure is a method for manufacturing a dry chemistry test strip, (DCD) for the determination of calcitonin in a test sample. Calcitonin is a hormone that has been linked to hypercalcenemia, the hormone rapidly lowers blood calcium by inhibiting bone resorption; it also increases urinary excretion of phosphate. Normal levels are 100 pg/mL or less. Higher levels are considered abnormal.

Filter paper is impregnated with the following solutions and dried at 25 degree C.:

Solution 1

2-[N-Morpholino]ethansulfonic Acid buffer (MES) 0.1 M

300 ng/mL Nucleounits targeted to calcitonin is conjugated to horseradish peroxidase

900 mL D.I. water, mix, adjust pH to 6.0, and Q.S. to 1000 mL with D.I. water

Solution 2

2-[N-Morpholino]ethansulfonic Acid buffer 0.1 M

Tetramethylbenzidine, (TMB) 500 mg

Urea-Peroxide, 5.0 g

900 mL D.I. water, mix, and adjust

pH between 5.0 and 7.0, preferably 6.0

Q.S. to 1000 mL with D.I. water

Nucleounits manufactured to be specific for calcitonin can conjugated to horseradish peroxidase; the techniques for producing these types of conjugations is well known in the art.

This assay uses a nucleounit to calcitonin which is then conjugated to peroxidase. When the nucleounit which is conjugated to the peroxidase binds to its target calcitonin, it releases the peroxidase which is then free to react with peroxide and the chromogen, TMB, resulting in formation of a blue-green colored complex. This color reaction yields a visible color change. Therefore, the calcitonin concentration is proportional to the intensity of the blue-green color produced.

The test device in this example is manufactured in much the same manner as that in Example 7 and the solutions are impregnated onto the paper in the exact same manner. If this device is constructed using two reaction pads, the reaction pad containing solution 2 must be on the bottom half of the “sandwich”. In addition, it may be necessary to separate the two pads with a semipermeable membrane.

Therefore, the production of this test device is carried out using the following procedure. The test device, a piece of Whatman 3 MM filter paper having dimensions of 0.25 inch by 3 inches is impregnated with solution 1 by immersion into it. The paper is then dried by using forced air not exceeding 60 degrees C. After drying the paper is then impregnated with solution 2 by immersion and dried by forced air not to exceed 60 degrees C. The paper is then cut into smaller pieces measuring 0.25 inches by 0.25 inches. The paper is then laminated to one side of a double-sided adhesive transfer tape commercially available from 3M Company, St. Paul, Minn. 55144. This laminate (paper plus adhesive) measures 0.25 inches by 0.25 inches. The laminate is then attached, via the unused adhesive side, to one end of a sturdy polystyrene strip measuring about 0.25 inches by 3 inches; the resulting product forms a test device comprising a 3 inch long polystyrene handle with a square of the impregnated test paper at one end. The dipstick thus obtained will produce a greenish to blue color when exposed to calcitonin at a concentration of equal to or greater than 100 pg/mL. In fact, the intensity of the color is proportional to the concentration of calcitonin present in the sample. The test device, therefore, effectively measures the presence and concentration of calcitonin in serum.

To summarize the example more specifically, the foregoing dry chemistry test strip (DCD) method to measure calcitonin concentration in a serum sample, the method comprising the steps of preparing a test means by successively impregnating a carrier matrix with reagent solutions containing nucleounits targeted to calcitonin, drying said test means, dipping completed test means into a test sample, and determining the quantity of calcitonin present in said test sample by comparing the relative intensity and color produced by the reaction to a color chart with color blocks referenced to specific concentrations of calcitonin.

As described however, an additional solution is required. This solution 2 is incorporated into the test device by immersing the test paper into solution 2; the paper is then dried by using forced air not exceeding 60 degrees C. as previously described. A two-part test pad “sandwich” could be used and in that case the pad with solution #1 must be on top and the pad with solution 2 is on the bottom. The dipstick thus obtained will produce a green to bluish color when exposed to calcitonin at a concentration of 100 pg/mL or greater. In fact, the intensity of the color is proportional to the concentration of calcitonin in the sample. This test device, therefore, effectively identifies calcitonin in serum or other biological matrices using a nucleounit manufactured to be specifically targeted to calcitonin.

Changes to the foregoing solutions could be made and still have similar results. The foregoing solutions could be combined together, or reduced to only 1. The concentrations of said constituents may also be changed and still remain within the scope of the invention. The urea peroxide was chosen, because it is more stable than simple peroxide. It is obvious, however, that one may utilize any peroxide-containing compound to act as a substrate to peroxidase.

The TMB may be replaced by any suitable compound that will produce an observable color as part of the peroxidase/peroxide reaction. Other such compounds include ABTS (2,2′-Azino-di-(3-ethylbenzthiazolinesulfonic acid) diammonium salt, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), N,N-Bis(4-sulfobutyl)-3,5-dimethylaniline (MADB), and pyrogallol. Also, 4-aminoantipyrine can be paired with a number of compounds to create a violet to violet-blue color complex in the presence of the peroxide/peroxidase reaction. These compounds include 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, and N-Ethyl-N-(2-hydroxy-3-Sulfopropyl)-m-toluidine. Another indicator pair that may be utilized consists of 3-Methyl-2-benzothiazolinonehydrazone and Dimerhylaniline.

In addition, it is possible to conjugate other enzymes to nucleounits. Consequently, these conjugated pairs can also be substituted into the test reaction together with an appropriate indicator compound. Therefore, this assay may include any enzyme capable of being conjugated to a nucleounit.

To further describe the preferred test method for determining calcitonin in an unknown test sample, the assay system can take the form of a dipstick (DCD), lateral flow device (LFD), or an aqueous liquid reagent that is composed of a buffer and an indicator that produces a color or change in the intensity of color or absorbance in the UV or visible spectrum in the presence of calcitonin.

This art as taught in previous examples can also employ a dry chemistry test strip (DCD) method for measuring the calcitonin concentration in a test sample, the method comprising the steps of preparing a test means by successively impregnating a carrier matrix with reagent solutions, drying said test means, dipping completed test means into test sample, and determining the quantity of calcitonin nucleounit targeted markers present in said test sample by comparing the relative intensity of the color produced by the reaction to a color chart with color blocks referenced to specific concentrations of calcitonin. The above described assay method may also be applied to other nucleounit targets.

This art as taught in previous examples can also employ a dry chemistry, lateral flow device (LFD) for measuring the calcitonin concentration in a test sample, the method comprising the steps of preparing a test means by successively impregnating a solid, absorbent carrier matrix with liquid, reagent solutions at specific locations on the test means, drying said test means, dipping completed test means into test sample or pipetting test sample onto the test means, and determining the quantity of calcitonin in the test sample by comparing the relative intensity (completeness) of the assay line produced by the reaction to a standard chart, or by comparing the relative intensity (completeness) of the assay line produced by the reaction to the control line. The above described assay method may also be applied to other nucleounit targets.

Changes to the foregoing solutions could be made and still have similar results. The concentrations of said constituents may also be changed and still remain within the scope of the invention. The nucleounit to calcitonin is produced per the manufacturing procedure for manufacturing nucleounits as previously described. It should be noted that the production of the nucleounit to calcitonin did not require the use of any animals and is as specific or more than an antibody to calcitonin.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

Manufacture of the dry chemistry dipsticks may require the addition of thickeners as taught in the art. Some compounds commonly used for this purpose include: polyvinylpyrrolidone, algin, carragenin, casein, albumin, methyl cellulose, and gelatin. The typical range of concentration for these thickeners is about 0.5 to 5.0 g. per 100 ml. Wetting agents or surfactants are also typically used in dry chemistry. For dry chemistry applications, wetting agents aid in even distribution of the chemicals and promote even color development. Acceptable wetting agents can be hydrophilic polymers, or cationic, anionic, amphoteric, or nonionic species. Some commonly used wetting agents include sodium dodecyl-benzene sulphonate, sodium lauryl sulphate, benzalkonium chloride, N-lauroylsarcosine sodium salt, Brij-35, Tween 20, Triton X-100, dioctyl sodium sulphosuccinate, and polyethylene glycol 6000. Wetting agents can be added to dipstick impregnation solutions in amounts of 0.5% to 5.0%, and 0.1% to 1.0% in liquid reagents.

Color enhancers may be used such as sucrose, lactose, glucose or other compounds. Color enhancement can be defined as intensification and/or alteration in some manner the color that is produced by the reaction to improve the measurement of the detectable response.

The production of dry chemistry test strips for the present invention can utilize any form of absorbent, solid phase carrier including filter paper, cellulose or synthetic resin fleeces in conjunction with liquid solutions of reagent compositions in volatile solvents. This can be carried out in one or more impregnation steps. Each impregnation may contain one or more of the chemical compounds making up the assay reagent composition; the exact procedure is dictated by the inter-reactivity of the assay constituents and the order in which they may have to react with the analyte of interest.

In the case of the DLFH or LFD of the present invention it can utilize any form of absorbent, solid phase carrier that is capable of transporting a fluid. These can include filter paper, cellulose or synthetic resins. More specifically, the lateral flow material can include cellulose, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

After impregnation, the dipsticks are dried, cut into strips, glued to a support structure (usually a flexible, flat, plastic stick made up of polystyrene, vinyl polypropylene, and polyester or other suitable support material) as part of a “sandwich” composed of the handle, test pad, and a synthetic resin film and/or a fine-mesh material in the manner described in German Pat. No, 2,118,455. In addition, the instant invention may be combined with the water-stable film as taught in U.S. Pat. No. 3,530,957 to produce a dipstick in which the excess sample fluid can be wiped off in order to improve the accuracy and precision of the results.

### EXAMPLE 9

The following procedure is a method for manufacturing a dry chemistry test strip, (DCD) for the determination of gentamicin in a test sample. Gentamicin is an antibiotic. Therapeutic ranges for infection are from 5 to 10 ug/mL. Greater than 10 ug/mL is considered toxic.

Filter paper is impregnated with the following solutions and dried at 25 degree C.:

Solution 1

0.05 M Pipes (Piperazine-N-N′-bis[2-ethanesulfonic acid];

1,4-piperazinediethanesulfonic acid)

300 ng/mL Nucleounits targeted to calcitonin is conjugated to horseradish peroxidase

900 mL D.I. water, mix, adjust pH to 6.8, and Q.S. to 1000 mL with D.I. water

Solution 2

0.05 M Pipes (Piperazine-N-N′-bis[2-ethanesulfonic acid];

1,4-piperazinediethanesulfonic acid)

500 mg ABTS (2,2′-Azino-di-(3-ethylbenzthiazolinesulfonic acid) diammonium salt Urea-Peroxide, 5.0 g

900 mL D.I. water, mix, and adjust

pH between 5.0 and 7.0, preferably 6.8

Q.S. to 1000 mL with D.I. water

Nucleounits manufactured to be specific for gentamicin can conjugated to horseradish peroxidase; the techniques for producing these types of conjugations is well known in the art.

This assay utilizes a nucleounit to gentamicin which is then conjugated to peroxidase. When the nucleounit which is conjugated to the peroxidase binds to its target gentamicin, it releases the peroxidase which is then free to react with peroxide and the chromogen, ABTS, resulting in formation of a blue-green colored complex. This color reaction yields a visible color change. Therefore, the gentamicin concentration is proportional to the intensity of the blue-green color produced.

The test device in this example is manufactured in the much same manner as that in example 8 and the solutions are impregnated onto the paper in the exact same manner. If this device is constructed using two reaction pads, the reaction pad containing solution 2 must be on the bottom half of the “sandwich”. In addition, it may be necessary to separate the two pads with a semipermeable membrane.

Production of this test device is carried out using the following procedure. The test device, a piece of Whatman 3 MM filter paper having dimensions of 0.25 inch by 3 inches is impregnated with solution 1 by immersion into it. The paper is then dried by using forced air not exceeding 60 degrees C. After drying the paper is then impregnated with solution 2 by immersion and dried by forced air not to exceed 60 degrees C. The paper is then cut into smaller pieces measuring 0.25 inches by 0.25 inches. The paper is then laminated to one side of a double-sided adhesive transfer tape commercially available from 3M Company, St. Paul, Minn. 55144. This laminate (paper plus adhesive) measures 0.25 inches by 0.25 inches. The laminate is then attached, via the unused adhesive side, to one end of a sturdy polystyrene strip measuring about 0.25 inches by 3 inches; the resulting product forms a test device comprising a 3 inch long polystyrene handle with a square of the impregnated test paper at one end. The dipstick thus obtained will produce a greenish to blue color when exposed to gentamicin at a concentration of equal to or greater than 10 ug/mL. In fact, the intensity of the color is proportional to the concentration of gentamicin present in the sample. The test device, therefore, effectively measures the presence and concentration of gentamicin in serum.

To summarize the example more specifically, the foregoing dry chemistry test strip (DCD) method to measure gentamicin concentration in a serum sample, the method comprising the steps of preparing a test means by successively impregnating a carrier matrix with reagent solutions containing nucleounits targeted to gentamicin, drying said test means, dipping completed test means into a test sample, and determining the quantity of gentamicin present in said test sample by comparing the relative intensity and color produced by the reaction to a color chart with color blocks referenced to specific concentrations of gentamicin.

As described however, an additional solution is required. This solution 2 is incorporated into the test device by immersing the test paper into solution 2; the paper is then dried by using forced air not exceeding 60 degrees C. as previously described. A two-part test pad “sandwich” could be used and in that case the pad with solution #1 must be on top and the pad with solution 2 is on the bottom. The dipstick thus obtained will produce a greenish to blue color when exposed to gentamicin at a concentration of 10 ug/mL or greater. In fact, the intensity of the color is proportional to the concentration of gentamicin in the sample. This test device, therefore, effectively identifies gentamicin in serum or other biological matrices using a nucleounit manufactured to be specifically targeted to gentamicin.

Changes to the foregoing solutions could be made and still have similar results. The foregoing solutions could be combined together, or reduced to only 1. The concentrations of said constituents may also be changed and still remain within the scope of the invention. The urea peroxide was chosen, because it is more stable than simple peroxide. It is obvious, however, that one may utilize any peroxide-containing compound to act as a substrate to peroxidase.

The ABTS (2,2′-Azino-di-(3-ethylbenzthiazolinesulfonic acid) diammonium salt may be replaced by any suitable compound that will produce an observable color as part of the peroxidase/peroxide reaction. Other such compounds include Tetramethylbenzidine (TMB), AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), N,N-Bis(4-sulfobutyl)-3,5-dimethylaniline (MADB), and pyrogallol. Also, 4-aminoantipyrine can be paired with a number of compounds to create a violet to violet-blue color complex in the presence of the peroxide/peroxidase reaction. These compounds include 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, and N-Ethyl-N-(2-hydroxy-3-Sulfopropyl)-m-toluidine. Another indicator pair that may be utilized consists of 3-Methyl-2-benzothiazolinonehydrazone and Dimerhylaniline.

In addition, it is possible to conjugate other enzymes to nucleounits. Consequently, these conjugated pairs can also be substituted into the test reaction together with an appropriate indicator compound. Therefore, this assay may include any enzyme capable of being conjugated to a nucleounit.

To further describe the preferred test method for determining gentamicin in an unknown test sample, the assay system can take the form of a dipstick (DCD), lateral flow device (LFD), or an aqueous liquid reagent that is composed of a buffer and an indicator that produces a color or change in the intensity of color or absorbance in the UV or visible spectrum in the presence of gentamicin.

This art as taught in previous examples can also employ a dry chemistry test strip (DCD) method for measuring the calcitonin concentration in a test sample, the method comprising the steps of preparing a test means by successively impregnating a carrier matrix with reagent solutions, drying said test means, dipping completed test means into test sample, and determining the quantity of osteoporosis antigen markers present in said test sample by comparing the relative intensity of the color produced by the reaction to a color chart with color blocks referenced to specific concentrations of calcitonin. The above described assay method may also be applied to other nucleounit targets.

This art as taught in previous examples can also employ a dry chemistry, lateral flow device (LFD) for measuring the gentamicin concentration in a test sample, the method comprising the steps of preparing a test means by successively impregnating a solid, absorbent carrier matrix with liquid, reagent solutions at specific locations on the test means, drying said test means, dipping completed test means into test sample or pipetting test sample onto the test means, and determining the quantity of gentamicin in the test sample by comparing the relative intensity (completeness) of the assay line produced by the reaction to a standard chart, or by comparing the relative intensity (completeness) of the assay line produced by the reaction to the control line. The above described assay method may also be applied to other nucleounit targets.

Again, changes to the foregoing solutions could be made and still have similar results. The concentrations of said constituents may also be changed and still remain within the scope of the invention. The nucleounit to gentamicin is produced per the manufacturing procedure for manufacturing nucleounits as previously described. It should be noted that the production of the nucleounit to gentamicin did not require the use of any animals and is as specific or more than an antibody to gentamicin.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), 2-[N-Morpholino]ethansulfonic Acid buffer (MES), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml. Optional surfactants may include long chain organic sulphates or sulphonates (e.g. Brij-35, Tween 20, Triton X-100, dioctyl sodium sulphosuccinate, and sodium lauryl sulphate).

Manufacture of the dry chemistry dipsticks may require the addition of thickeners as taught in the art. Some compounds commonly used for this purpose include: polyvinylpyrrolidone, algin, carragenin, casein, albumin, methyl cellulose, and gelatin. The typical range of concentration for these thickeners is about 0.5 to 5.0 g. per 100 ml. Wetting agents or surfactants are also typically used in dry chemistry. For dry chemistry applications, wetting agents aid in even distribution of the chemicals and promote even color development. Acceptable wetting agents can be hydrophilic polymers, or cationic, anionic, amphoteric, or nonionic species. Some commonly used wetting agents include sodium dodecyl-benzene sulphonate, sodium lauryl sulphate, benzalkonium chloride, N-lauroylsarcosine sodium salt, Brij-35, Tween 20, Triton X-100, dioctyl sodium sulphosuccinate, and polyethylene glycol 6000. Wetting agents can be added to dipstick impregnation solutions in amounts of 0.5% to 5.0%, and 0.1% to 1.0% in liquid reagents.

Color enhancers may be used such as sucrose, lactose, glucose or other compounds. Color enhancement can be defined as intensification and/or alteration in some manner the color that is produced by the reaction to improve the measurement of the detectable response.

The production of dry chemistry test strips for the present invention can utilize any form of absorbent, solid phase carrier including filter paper, cellulose or synthetic resin fleeces in conjunction with liquid solutions of reagent compositions in volatile solvents. This can be carried out in one or more impregnation steps. Each impregnation may contain one or more of the chemical compounds making up the assay reagent composition; the exact procedure is dictated by the inter-reactivity of the assay constituents and the order in which they may have to react with the analyte of interest.

In the case of the DLFH or LFD of the present invention it can utilize any form of absorbent, solid phase carrier that is capable of transporting a fluid. These can include filter paper, cellulose or synthetic resins. More specifically, the lateral flow material can include cellulose, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

After impregnation, the dipsticks are dried, cut into strips, glued to a support structure (usually a flexible, flat, plastic stick made up of polystyrene, vinyl polypropylene, and polyester or other suitable support material) as part of a “sandwich” composed of the handle, test pad, and a synthetic resin film and/or a fine-mesh material in the manner described in German Pat. No, 2,118,455. In addition, the instant invention may be combined with the water-stable film as taught in U.S. Pat. No. 3,530,957 to produce a dipstick in which the excess sample fluid can be wiped off in order to improve the accuracy and precision of the results.

### EXAMPLE 10

This example will illustrate in detail the exact method for manufacturing the lateral flow GHB method using nucleounits targeted specifically to GHB. Keep in mind this method could be utilized for any general chemistry “test pad” or pads that are currently used or will be used in the art. In the case of DLFH technology, the manufacturing process includes impregnating onto an absorbent, solid carrier (e.g. paper) called in this example, the “test pad”, in exactly the same manner as Examples 8 or 9. The test pad, once impregnated, is dried, then mounted onto a solid support (nitrocellulose membrane) that is capable of transporting (through lateral flow) liquid to the test pad from the point of application of a test sample. In simpler terms, the device is dipped into a liquid or the liquid sample is placed on the device at the bottom or starting point for the assay. The liquid migrates from the starting application point to the opposite end of the nitrocellulose lateral flow paper, during which the test pad becomes saturated with the sample. The reaction takes place on the test pad and color develops. The developed color is then compared to a color chart with known concentrations of GHB that has the appropriate colors relative to each specific concentration of GHB(s). For example a specific color for 0.0 ug/mL GHB, 50.0 ug/mL, 100.0 ug/mL, etc., for comparison. The results are then recorded. Note, the test pad must be an absorbent (wicking) material that permits migration of sample up the solid absorbent test pad and allows analytes and reactants to interact.

Absorbent material is successively impregnated with the following solutions and dried at 25 degree C.:

Solution 1

NAD 0.3 M

Nucleounit targeted to GHB conjugated to hydroxybutyrate dehydrogenase 5 mM

Tris-HCl (Tris[hydroxymethyl]aminomethane hydrochloride) buffer 0.01M

NBT (nitro blue tetrazolium) 0.01 mg/L

distilled water added to 100 mL total volume of solution

pH the solution to a value between 1.0 and 12.5 preferably 8.0

lab notes: Buffer strength is preferably 10 mM or greater

In this example, the lateral flow device is prepared in accordance with the instant invention. The lateral flow device is comprised of a paper carrier matrix (S&S, 593 grade filter paper) impregnated with the compositions of solution 1 and dried by forced air. The paper is then cut into test pads 5 mm by 5 mm. Note that said concentrations of any of the above constituents can be varied to suit the DLFH lateral flow/dipstick device format (e.g. dependent upon paper type, and inclusion of semi-permeable membranes or other innovations utilized in dry chemistry technology). The dried impregnated test pad is then placed at approximately 35 mm (in the middle) of a 5 mm wide by 70 mm long nitrocellulose membrane (S&S FastTrack™ NC) and makes fluid contact with nitrocellulose lateral flow paper. The nitrocellulose membrane is capable of transporting a liquid by capillary action or wicking from one end of the lateral flow device to the other in approximately 60 seconds. In this example, the DLFH has the dimensions of 5 mm wide by 70 mm long and can be backed by or in contact with strips of glass fiber filter material (e.g. S&S 30 grade) to aid in controlling the wicking action, or other solid support material can be used.

Again, to completely illustrate the present device the starting point or origin at which the sample is placed on the test device is 5 mm from one end of the strip, and 35 mm from the site of where the test pad is placed in fluid contact with the strip. For simplicity, this example will have the 5 mm by 5 mm impregnated test pad placed on top of the lateral flow paper and thus be in fluid contact with the said paper.

The mechanics of how the present art's LFD and dipstick test pad hybrid may be explained is as follows. The starting point or origin at which the sample is placed on the test device is 5 mm from one end of the strip, and 35 mm from site where the chemically impregnated test pad is in fluid contact with the lateral flow paper. The test pad can be placed on top of the lateral flow paper making fluid juxtaposed) contact with the lateral flow paper from the bottom side of the test pad, or the lateral flow paper can touch the paper from the side of the test pad and remain in fluid contact with the test pad. Or the lateral flow paper can rest on top of the edge of test pad or be attached and in fluid contact with the test pad in some other manner. One of the novel advantages in using a hybrid device made of lateral flow material and a dry chemistry test pad is the lack of cross contamination from one pad to the next from excessive fluid, as is inherent in the prior art. For illustration, currently there are available many different types of dry chemistry test strips available, such as the Miles Laboratories, Inc. MULTISTIXref. This device and many other like it has multiple reagents test pads with different chemistries impregnated onto each pad on a single support membrane backing (usually plastic). Because of the relative proximity of these pads to each other on the same device it is easy for cross contamination to occur, causing unreliable results. This is called “runover” (i.e. when a reagent from one pad runs over another adjacent test pad). The present arts eliminates runover. The applicant's novel approach to the solution of runover has not been taught prior to the present art and is the result of extensive research and development.

Result interpretation can be explained as follows. If the sample is positive, with a concentration of 50.0 ug/mL GHB or more, the following occurs. A drop of urine (approximately 50 uL) is applied at the starting point or origin of the strip. The urine then migrates to the opposite or terminal end of the strip. As the urine migrates across the lateral flow material (nitrocellulose) and comes into contact with the test pad (filter paper), the urine will saturate the pad and cause a chemical reaction between the impregnated chemicals containing nucleounits to GHB and GHB in the urine. A blue color will develop on the test pad indicating a positive (greater than 50.0 ug/mL GHB) for the presence of high levels of GHB. This color can then be compared to a color chart showing the different colors from colorless (white background)) to a dark blue depending upon the concentration of the GHB(s), if greater than 50.0 ug/mL. The reaction on the test pad is immediate thus the test results can be observed immediately.

If the sample is negative, with a concentration of less than 50.0 ug/mL of GHB present the following occurs. A drop of urine (approximately 50 uL) is applied at the starting point or origin of the strip. The urine then migrates to the opposite or terminal end of the strip. As the urine migrates across the lateral flow material and comes into contact with the test pad, the urine will saturate the pad and cause a chemical reaction between the impregnated chemicals and GHB. However, this example is for a negative result, thus, no reaction occurs and no color develops, indicating a negative result. This negative result color can then be compared to a color chart showing the different colors from no color developed (negative) to dark blue depending upon the concentration of the GHB, if greater than 50 ug/mL GHB. The reaction on the test pad is immediate thus the test results can be observed immediately.

Changes to the above reagent solution of this example can be made and still remain within the scope and function of this invention and will have similar results.

This brief description of the present art illustrates a completely enabled device that would allow an individual, physician, patient, and/or technician to quickly and easily determine the presence of the GHB in urine, providing a much needed advancement the art of GHB testing.

To briefly explain the present device as taught. The present art includes a device for the detection of GHB in a sample of urine submitted for drugs of abuse testing the steps comprise of preparing a dry chemistry test means by successively impregnating a solid, carrier matrix with reagent solutions containing a nucleounit specific for GHB conjugated to an indicator and a buffer, and drying the impregnated, solid carrier matrix. Finally, by dipping said dry chemistry test means into urine, one can observe the detectable response in the form of a color developed in the presence or absence of GHB. This present art also illustrates a unique device that will prevent cross contamination (runover) of test pads on the same dipstick, as well as a unique dry chemistry test pad lateral flow device hybrid. These methods can incorporate detectable responses in the visible color range to the human eye or in the visible light spectrum. These methods have a wide sample choice other than urine, and can be replaced by any biological sample including serum, whole blood, cerebral spinal fluid, gastric fluid, hair homogenates, sweat extracts, saliva or other biological fluid and other fluids such as water, beverages (beer, soft drinks, etc.), to include alcohol drinks.

The concentrations of said constituents may also be changed and still remain within the scope of the invention. The nucleounit to GHB is produced per the manufacturing procedure for manufacturing nucleounits as previously described. It should be noted that the production of the nucleounit to GHB did not require the use of any animals and is as specific or more than an antibody to GHB.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, MES (2-[N-Morpholino]ethanesulfonic acid), BIS-TRIS (bis[2-Hydroxyethylaiminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), 2-[N-Morpholino]ethansulfonic Acid buffer (MES), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml.

Manufacture of the dry chemistry dipsticks may require the addition of thickeners as taught in the art. Some compounds commonly used for this purpose include: polyvinylpyrrolidone, algin, carragenin, casein, albumin, methyl cellulose, and gelatin. The typical range of concentration for these thickeners is about 0.5 to 5.0 g. per 100 ml. Wetting agents or surfactants are also typically used in dry chemistry. For dry chemistry applications, wetting agents aid in even distribution of the chemicals and promote even color development. Acceptable wetting agents can be hydrophilic polymers, or cationic, anionic, amphoteric, or nonionic species. Some commonly used wetting agents include sodium dodecyl-benzene sulphonate, sodium lauryl sulphate, benzalkonium chloride, N-lauroylsarcosine sodium salt, Brij-35, Tween 20, Triton X-100, dioctyl sodium sulphosuccinate, and polyethylene glycol 6000. Wetting agents can be added to dipstick impregnation solutions in amounts of 0.5% to 5.0%, and 0.1% to 1.0% in liquid reagents.

Color enhancers may be used such as sucrose, lactose, glucose or other compounds. Color enhancement can be defined as intensification and/or alteration in some manner the color that is produced by the reaction to improve the measurement of the detectable response.

The production of dry chemistry test strips for the present invention can utilize any form of absorbent, solid phase carrier including filter paper, cellulose or synthetic resin fleeces in conjunction with liquid solutions of reagent compositions in volatile solvents. This can be carried out in one or more impregnation steps. Each impregnation may contain one or more of the chemical compounds making up the assay reagent composition; the exact procedure is dictated by the inter-reactivity of the assay constituents and the order in which they may have to react with the analyte of interest.

In the case of the DLFH or LFD of the present invention it can utilize any form of absorbent, solid phase carrier that is capable of transporting a fluid. These can include filter paper, cellulose or synthetic resins. More specifically, the lateral flow material can include cellulose, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

After impregnation, the dipsticks are dried, cut into strips, glued to a support structure (usually a flexible, flat, plastic stick made up of polystyrene, vinyl polypropylene, and polyester or other suitable support material) as part of a “sandwich” composed of the handle, test pad, and a synthetic resin film and/or a fine-mesh material in the manner described in German Pat. No, 2,118,455. In addition, the instant invention may be combined with the water-stable film as taught in U.S. Pat. No. 3,530,957 to produce a dipstick in which the excess sample fluid can be wiped off in order to improve the accuracy and precision of the results.

### EXAMPLE 11

This example will illustrate in detail the exact method for manufacturing the lateral flow opiates method using nucleounits targeted specifically to opiates. Keep in mind this method could be utilized for any general chemistry “test pad” or pads that are currently used or will be used in the art. In the case of DLFH technology, the manufacturing process includes impregnating onto an absorbent, solid carrier (e.g. paper) called in this example, the “test pad”, in exactly the same manner as Examples 8 or 9. The test pad, once impregnated, is dried, then mounted onto a solid support (nitrocellulose membrane) that is capable of transporting (through lateral flow) liquid to the test pad from the point of application of a test sample. In simpler terms, the device is dipped into a liquid or the liquid sample is placed on the device at the bottom or starting point for the assay. The liquid migrates from the starting application point to the opposite end of the nitrocellulose lateral flow paper, during which the test pad becomes saturated with the sample. The reaction takes place on the test pad and color develops. The developed color is then compared to a color chart with known concentrations of opiates that has the appropriate colors relative to each specific concentration of opiate(s). For example a specific color for 0.0 ug/mL opiates, 50.0 ug/mL, 100.0 ug/mL, etc., for comparison. The results are then recorded. Note, the test pad must be an absorbent (wicking) material that permits migration of sample up the solid absorbent test pad and allows analytes and reactants to interact.

Absorbent material is successively impregnated with the following solutions and dried at 25 degree C.:

Solution 1

NAD 0.3 M

7.0 mM Nucleounit targeted to opiates conjugated to G6PD (glucose-6-phosphate dehydrogenase)

0.05 M Hepes

NBT (nitro blue tetrazolium) 0.01 mg/L

distilled water added to 100 nL total volume of solution

pH the solution to a value between 1.0 and 12.5 preferably 7.5

lab notes: Buffer strength is preferably 10 mM or greater

In this example, the lateral flow device is prepared in accordance with the instant invention. The lateral flow device is comprised of a paper carrier matrix (S&S, 593 grade filter paper) impregnated with the compositions of solution 1 and dried by forced air. The paper is then cut into test pads 5 mm by 5 mm. Note that said concentrations of any of the above constituents can be varied to suit the DLFH lateral flow/dipstick device format (e.g. dependent upon paper type, and inclusion of semi-permeable membranes or other innovations utilized in dry chemistry technology). The dried impregnated test pad is then placed at approximately 35 mm (in the middle) of a 5 mm wide by 70 mm long nitrocellulose membrane (S&S FastTrack™ NC) and makes fluid contact with nitrocellulose lateral flow paper. The nitrocellulose membrane is capable of transporting a liquid by capillary action or wicking from one end of the lateral flow device to the other in approximately 60 seconds. In this example, the DLFH has the dimensions of 5 mm wide by 70 mm long and can be backed by or in contact with strips of glass fiber filter material (e.g. S&S 30 grade) to aid in controlling the wicking action, or other solid support material can be used.

Again, to completely illustrate the present device the starting point or origin at which the sample is placed on the test device is 5 mm from one end of the strip, and 35 mm from the site of where the test pad is placed in fluid contact with the strip. For simplicity, this example will have the 5 mm by 5 mm impregnated test pad placed on top of the lateral flow paper and thus be in fluid contact with the said paper.

The mechanics of how the present art's LFD and dipstick test pad hybrid may be explained is as follows. The starting point or origin at which the sample is placed on the test device is 5 mm from one end of the strip, and 35 mm from site where the chemically impregnated test pad is in fluid contact with the lateral flow paper. The test pad can be placed on top of the lateral flow paper making fluid juxtaposed) contact with the lateral flow paper from the bottom side of the test pad, or the lateral flow paper can touch the paper from the side of the test pad and remain in fluid contact with the test pad. Or the lateral flow paper can rest on top of the edge of test pad or be attached and in fluid contact with the test pad in some other manner. One of the novel advantages in using a hybrid device made of lateral flow material and a dry chemistry test pad is the lack of cross contamination from one pad to the next from excessive fluid, as is inherent in the prior art. For illustration, currently there are available many different types of dry chemistry test strips available, such as the Miles Laboratories, Inc. MULTISTIXref . This device and many other like it has multiple reagents test pads with different chemistries impregnated onto each pad on a single support membrane backing (usually plastic). Because of the relative proximity of these pads to each other on the same device it is easy for cross contamination to occur, causing unreliable results. This is called “runover” (i.e. when a reagent from one pad runs over another adjacent test pad). The present arts eliminates runover. The applicant's novel approach to the solution of runover has not been taught prior to the present art and is the result of extensive research and development.

Result interpretation can be explained as follows. If the sample is positive, with a concentration of 300 ng/mL Opiates or more, the following occurs. A drop of urine (approximately 50 uL) is applied at the starting point or origin of the strip. The urine then migrates to the opposite or terminal end of the strip. As the urine migrates across the lateral flow material (nitrocellulose) and comes into contact with the test pad (filter paper), the urine will saturate the pad and cause a chemical reaction between the impregnated chemicals containing nucleounits to opiates and opiates in the urine. A blue color will develop on the test pad indicating a positive (greater than 300 g/mL opiates) for the presence of high (drug abuse positive) levels of opiates. This color can then be compared to a color chart showing the different colors from colorless (white background)) to a dark blue depending upon the concentration of the opiates, if greater than 300 ng/mL opiates. The reaction on the test pad is immediate thus the test results can be observed immediately.

If the sample is negative, with a concentration of less than 300 ng/mL opiates present the following occurs. A drop of urine (approximately 50 uL) is applied at the starting point or origin of the strip. The urine then migrates to the opposite or terminal end of the strip. As the urine migrates across the lateral flow material and comes into contact with the test pad, the urine will saturate the pad and cause a chemical reaction between the impregnated chemicals and opiates. However, this example is for a negative result, thus, no reaction occurs and no color develops, indicating a negative result. This negative result color can then be compared to a color chart showing the different colors from no color developed (negative) to dark blue depending upon the concentration of the opiates, if greater than 300 ng/mL opiates. The reaction on the test pad is immediate thus the test results can be observed immediately.

Changes to the above reagent solution of this example can be made and still remain within the scope and function of this invention and will have similar results.

This brief description of the present art illustrates a completely enabled device that would allow an individual, physician, patient, and/or technician to quickly and easily determine the presence of the opiates in urine, providing a much needed advancement the art of opiate testing.

To briefly explain the present device as taught. The present art includes a device for the detection of opiates in a sample of urine submitted for drugs of abuse testing the steps comprise of preparing a dry chemistry test means by successively impregnating a solid, carrier matrix with reagent solutions containing a nucleounit specific for opiates conjugated to an indicator and a buffer, and drying the impregnated, solid carrier matrix. Finally, by dipping said dry chemistry test means into urine, one can observe the detectable response in the form of a color developed in the presence or absence of opiates. This present art also illustrates a unique device that will prevent cross contamination (runover) of test pads on the same dipstick, as well as a unique dry chemistry test pad lateral flow device hybrid. These methods can incorporate detectable responses in the visible color range to the human eye or in the visible light spectrum. These methods have a wide sample choice other than urine, and can be replaced by any biological sample including serum, whole blood, cerebral spinal fluid, gastric fluid, hair homogenates, sweat extracts, saliva or other biological fluid and other fluids such as water, beverages (beer, soft drinks, etc.), to include alcohol drinks.

The concentrations of said constituents may also be changed and still remain within the scope of the invention. The nucleounit to opiates is produced per the manufacturing procedure for manufacturing nucleounits as previously described. It should be noted that the production of the nucleounit to opiates did not require the use of any animals and is as specific or more than an antibody to opiates.

A thorough search of patents and published research has revealed no relative art (i.e., prior art) even slightly resembling this technology. Other than the art of manual methods (ELISA and HPLC) described above, no similar chemical test means has been described prior to the disclosure of this method.

The buffer(s) used in this example may be substituted with one or more selected from the group consisting of: citrate, borate, phosphate, borax, sodium tetraborate decahydrate, sodium perchlorate, sodium chlorate, sodium carbonate, TRIS, MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), HEPES (N-[2-Hydroxyethyl]piperazine-N′-[2-ethanesulfonic acid]), TES (N-tris[Hydroxymethyl]methyl-2-aminomethanesulfonic acid; 2[2-Hysroxy-1,1-bis(hydroxymethyl)-ethyl]amino)ethanesulfonic acid), 2-[N-Morpholino]ethansulfonic Acid buffer (MES), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine, TRIS (Tris[hydroxymethyl]aminomethane), N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. Other buffers with an effective pK and pH range, and capacity suitable for maintaining the sample-reagent mixture within the required parameters of the assay's reaction mechanism may be added to the above group.

This Example's formulation could also include any one or more of the surfactants, thickeners, or interference-removing compounds disclosed above in this embodiment. Optional compounds for removal of interfering substances include mono, di, tri, and tetra sodium salts of EDTA or EGTA. Optional thickeners include polyvinylpyrrolidone, algin, carrageenin, casein, albumin, methyl cellulose, and gelatin in concentrations ranging from 0.5 to 5 g. per 100 ml.

Manufacture of the dry chemistry dipsticks may require the addition of thickeners as taught in the art. Some compounds commonly used for this purpose include: polyvinylpyrrolidone, algin, carragenin, casein, albumin, methyl cellulose, and gelatin. The typical range of concentration for these thickeners is about 0.5 to 5.0 g. per 100 ml. Wetting agents or surfactants are also typically used in dry chemistry. For dry chemistry applications, wetting agents aid in even distribution of the chemicals and promote even color development. Acceptable wetting agents can be hydrophilic polymers, or cationic, anionic, amphoteric, or nonionic species. Some commonly used wetting agents include sodium dodecyl-benzene sulphonate, sodium lauryl sulphate, benzalkonium chloride, N-lauroylsarcosine sodium salt, Brij-35, Tween 20, Triton X-100, dioctyl sodium sulphosuccinate, and polyethylene glycol 6000. Wetting agents can be added to dipstick impregnation solutions in amounts of 0.5% to 5.0%, and 0.1% to 1.0% in liquid reagents.

Color enhancers may be used such as sucrose, lactose, glucose or other compounds. Color enhancement can be defined as intensification and/or alteration in some manner the color that is produced by the reaction to improve the measurement of the detectable response.

The production of dry chemistry test strips for the present invention can utilize any form of absorbent, solid phase carrier including filter paper, cellulose or synthetic resin fleeces in conjunction with liquid solutions of reagent compositions in volatile solvents. This can be carried out in one or more impregnation steps. Each impregnation may contain one or more of the chemical compounds making up the assay reagent composition; the exact procedure is dictated by the inter-reactivity of the assay constituents and the order in which they may have to react with the analyte of interest.

In the case of the DLFH or LFD of the present invention it can utilize any form of absorbent, solid phase carrier that is capable of transporting a fluid. These can include filter paper, cellulose or synthetic resins. More specifically, the lateral flow material can include cellulose, cellulose acetate, nitrocellulose, mixed ester, teflon, polyvinylidene difluoride (PVDF), polytetrafluoroethylene (PTFE), polysulfone, cotton linter, non-woven rayon, glass fiber, nylon, ion exchange or other suitable membranes or solid support.

After impregnation, the dipsticks are dried, cut into strips, glued to a support structure (usually a flexible, flat, plastic stick made up of polystyrene, vinyl polypropylene, and polyester or other suitable support material) as part of a “sandwich” composed of the handle, test pad, and a synthetic resin film and/or a fine-mesh material in the manner described in German Pat. No, 2,118,455. In addition, the instant invention may be combined with the water-stable film as taught in U.S. Pat. No. 3,530,957 to produce a dipstick in which the excess sample fluid can be wiped off in order to improve the accuracy and precision of the results.

The following is a condensed version of the examples and teachings as explained in detail in this specification. The technology as taught is a method for detecting an analyte of interest using nucleounits targeted to said analyte of interest comprising: identifying a nucleounit from a mixture of synthetic random sequences of nucleounit libraries by the method comprising contacting the analyte of interest with said mixture, wherein the nucleounits have an affinity to the analyte of interest and bind the said analyte, then unbound nucleounits are removed by partitioning, then remaining nucleounits are amplified by polymerase chain reaction to obtain an enriched solution of nucleounits with high affinity for the analyte of interest, then nucleounits are then conjugated to indicator for the analyte of interest forming an nucleounit indicator conjugate, then detecting the analyte of interest using said nucleounit indicator conjugate in a buffer. Now with that said the nucleounit indicator conjugate can consist of a nucleounit conjugated to an indicator which can be selected from the group consisting of particles of any color and material including rubber, latex, plastics, synthetic solids, and metals, 4-Aminophenyl-beta-D-galactopyranoside, 3-indoxyl-beta-D-galactopyranoside (blue), 5-Bromo-4-chloro-3-indoxyl-beta-D-galactopyranoside (blue), 5-Bromo-3-indoxyl-beta-D-galactopyranoside (blue), 6-chloro-3-indoxyl-beta-D-galactopyranoside (salmon), 5-bromo-6-chloro-3-indoxyl-beta-D-galactopyranoside (Magenta-beta-D-Gal) 6-Fluoro-3-indoxyl-beta-D-galactopyranoside, 8-Hydroxyquinoline-beta-D-galactopyrano-side, 5-Iodo-3-indoxyl-beta-D-galactopyranoside (purple), 2-Nitrophenyl-beta-D-galactopyranoside, 4-Nitrophenyl-beta-D-galactopyranoside, Naphthol AS-BI-beta-D-galactopyranoside, 2-Naphthyl-beta-D-galactopyranoside (yellow), 4-Methylumbelliferyl-beta-D-glucuronic acid, galactosidase, beta-D-Galactosidase, Iodo-3-indoxyl-beta-D-galactopyranoside, alpha-L-Galactosidase, Iodo-3-indoxyl-alpha-L-galactopyranoside, beta-D-Cellobiosidase, 5-Bromo-4-chloro-3-indoxyl-beta-D-cellobioside, 5-Bromo-6-chloro-3-indoxyl-beta-D-cellobioside, 4-Nitrophenyl-beta-D-cellobioside, 1-Naphthyl-cellobioside, 4-Methylumbelliferyl-beta-D-cellobioside, glycosidases, galactosidase, gellobiosidase, Arabinosidase, Fucosidase, Galactosaminidase, Glucosaminidase, Glucosidase, Glucuronidase, Lactosidase, Maltosidase, Mannosidase, Xylosidase, arabinopyranoside, Fucopyranoside, Galactosaminide, Glucosaminide, Glucopyranoside, Glucuronic acid, Lactopyranoside, Maltopyranoside, Mannopyranoside, Xylopyranoside, 5-Bromo-4-chloro-3-indoxyl, 5-Bromo-6-chloro-3-indoxyl, 6-chloro-3-indoxyl, 5-Bromo-3-indoxyl, 5-Iodo-3-indoxyl, 3-indoxyl, 2-(6-Bromonaphthyl), 6-Fluoro-3-indoxyl 2-Nitrophenyl, 4-Nitrophenyl, 1-Naphthyl, Naphthyl AS-BI, 2-Nitrophenyl-N-acetyl, 4-Nitrophenyl-N-acetyl, 4-Methylumbelliferyl moieties, esterases, Carboxyl esterase, Cholesterol esterase, sulfatases, Aryl sufatase, phosphatases, Alkaline phosphatase, Carboxyl esterase, 6-chloro-3-indoxyl butyrate, Aryl sulfatase, 5-bromo-4-chloro-3-indoxyl sulfate, alkaline phosphatase 2-naphthyl, dehydrogenase, oxidase, hydroxylase, oxidoreductase, dehydrogenases, hydroxylases, a-NAD, nicotinamide adenine dinucleotide, nicotinamide adenine dinucleotide 3′-phosphate, nicotinamide adenine dinucleotide phosphate, triphosphopyridine, nicotinamide 1-N1-ethenoadenine dinucleotide phosphate, nicotinamide hypoxanthine dinucleotide, nicotinamide hypoxanthine dinucleotide phosphate, nicotinamide mononucleotide, nicotinamide N1-propylsulfonate, nicotinamide ribose monophosphate, Formaldehyde dehydrogenase, Fructose dehydrogenase, Glucose-6-phosphate dehydrogenase, Glucose dehydrogenase, Glutamate dehydrogenase, Glycerol dehydrogenase, Glycerol-3-phosphate dehydrogenase, hydroxybutyrate dehydrogenase, Hydroxybenzoate hydroxylase, Lactate dehydrogenase, Leucine dehydrogenase, Malate dehydrogenase, Mannitol dehydrogenase, oxidases, Acyl-CoA oxidase, alcohol oxidase, Ascorbate oxidase, Cholesterol oxidase, Choline oxidase, Glucose oxidase, Glycerophosphate oxidase, Xanthine oxidase, Uricase, peroxidase, pyrogallol, ABTS (2,2′-Azinobis(3-ethylbenzthiazoline) sulfonic acid), 3,3′,5,5′-Tetramethylbenzidine, ortho-Dianisidine, 3,3′-Diaminibenzidine, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline(DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), and N,N-Bis(4-sulfobutyl)-3,5-dimethylaniline (MADB), 3-Methyl-2-benzothiazolinonehydrazone, Dimerhylaniline, 4-aminoantipyrine, phenol, 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, N-Ethyl-N-(2-hydroxy-3-Sulfopropyl)-m-toluidine, NADPH oxidoreductase, NADPH, oxidoreductase, and NBT (nitro blue tetrazolium). The method also uses a buffer which can be selected from the group consisting of citrate, borate, phosphate, borax, sodium tetraborate decahydrate, sodium perchlorate, sodium chlorate, sodium carbonate, TRIS (Tris[hydroxymethyl]aminomethane), MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol) , ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), PIPES (PiperazineN-N′-bis[2-ethanesulfonic acid)]; 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), TES (N-tris[Hydroxymethyl]methyl-2-aminomethanesulfonic acid; 2[2-Hysroxy-1,1-bis(hydroxymethyl)-ethyl]amino)ethanesulfonic acid), HEPES (N-[2-Hydroxyethyl]piperazine-N′-[2-ethanesulfonic acid]), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine; N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAP SO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate. It is important to know that the nucleounits as described in this technology are not ligands, nucleic acid ligands or any other type of ligand. In fact it is quite the opposite. Ligands are molecules that bind proteins. The nucleounits in this sense are molecules that bind the molecules that may bind proteins which is the heart of the novelty of the discovery of this technology. This invention and the discoveries taught within are that the nucleounits are designed to detect for instance cocaine. This not a basic protein or even a complex protein. This is not an assay designed to detect complex proteins such as oligonucleotides but is used to detect analytes such as drugs-of-abuse, therapeutic drugs, antibiotics, autoimmune diseases, and infectious diseases to name a few and at best small sections of opposing nucleotides. In addition this technology as taught does not require a nucleounit indicator conjugate to be bound to a solid support.

The subject invention provides an extraordinary and novel method for quantitating the presence of analytes of interest using nucleounits in a biological specimen (i.e. urine, blood, serum, saliva, hair and sweat extracts, and cerebrospinal fluid) or other fluids.

In addition, the absolute novelty of creatinine measurement by the use of a DCD, LFD or DLFH is of enormous value to medical diagnostics and the health of our population. The utility of the present device when applied to aqueous, liquid form and modified for use on automated clinical chemistry analyzers is also of great value for the same reasons. All in all, the ability of the present art to analyze samples for analytes of interest using nucleounits via DCD, LFD, DLFH and aqueous, liquid reagent as described herein is a substantial and significant improvement over the prior art.

The synthetic nucleounit technology of the present art therefore, can prevent the injury and loss of life due to this disease, can be used to design novel and specific assay for the detection of analytes of interest without the use of animals or animal cell lines. The present art use of DCD, DLFH or LFD techniques provides a method for the general consumer (patient) to save money and still receive the health care needed by providing a test result for dollars at home or in the clinic versus the current art which costs hundreds of dollars. Ultimately this could save the consumer, nation and world economy millions of dollars. The clinical treatment and diagnostics of detection of diseases such as AIDS, CMV, Hepatitis are very expensive and time consuming as is well known in the art. Early detection of this diseases, and optimization of treatment is imperative to save dollars and lives. The ability to design and use nucleounits for the diagnosis of disease and drug-of-abuse testing as well as the many other modes as taught in this specification is very important when no loss of life, animals or the cruelty to animals was involved. The present society's technology has advanced greatly, but without the use of the present arts nucleounits animals will still used for the manufacturing and development of diagnostic kits, assays, and vaccines.

It will be understood from the embodiments that a person skilled in the art may make variations and modifications without departing from the spirit and scope of the invention. All such modifications and variations are to be included within the scope of the invention as defined in the appended claims.

I claim:

1. A method for detecting an analyte of interest using nucleounits targeted to said analyte of interest comprising:
a) identifying a nucleounit from a mixture of synthetic random sequences of nucleounit libraries, by the method comprising
i) contacting the analyte of interest with said mixture, wherein the nucleounits have an affinity to the analyte of interest and bind the said analyte;
ii) the unbound nucleounits are removed by partitioning;
iii) the remaining nucleounits are amplified by polymerase chain reaction to obtain an enriched solution of nucleounits with high affinity for the analyte of interest;
b) nucleounits are then conjugated to indicator for the analyte of interest forming an nucleounit indicator conjugate;
c) detecting the analyte of interest using said nucleounit indicator conjugate in a buffer.

2. The method according to claim 1 wherein said nucleounit indicator conjugate consist of a nucleounit conjugated to an indicator which can be selected from the group consisting of particles of any color and material including rubber, latex, plastics, synthetic solids, and metals, 4-Aminophenyl-beta-D-galactopyranoside, 3-indoxyl-beta-D-galactopyranoside (blue), 5-Bromo-4-chloro-3-indoxyl-beta-D-galactopyranoside (blue), 5-Bromo-3-indoxyl-beta-D-galactopyranoside (blue), 6-chloro-3-indoxyl-beta-D-galactopyranoside (salmon), 5-bromo-6-chloro-3-indoxyl-beta-D-galactopyranoside (Magenta-beta-D-Gal) 6-Fluoro-3-indoxyl-beta-D-galactopyranoside, 8-Hydroxyquinoline-beta-D-galactopyrano-side, 5-Iodo-3-indoxyl-beta-D-galactopyranoside (purple), 2-Nitrophenyl-beta-D-galactopyranoside, 4-Nitrophenyl-beta-D-galactopyranoside, Naphthol AS-BI-beta-D-galactopyranoside, 2-Naphthyl-beta-D-galactopyranoside (yellow), 4-Methylumbelliferyl-beta-D-glucuronic acid, galactosidase, beta-D-Galactosidase, Iodo-3-indoxyl-beta-D-galactopyranoside, alpha-L-Galactosidase, Iodo-3-indoxyl-alpha-L-galactopyranoside, beta-D-Cellobiosidase, 5-Bromo-4-chloro-3-indoxyl-beta-D-cellobioside, 5-Bromo-6-chloro-3-indoxyl-beta-D-cellobioside, 4-Nitrophenyl-beta-D-cellobioside, 1-Naphthyl-cellobioside, 4-Methylumbelliferyl-beta-D-cellobioside, glycosidases, galactosidase, gellobiosidase, Arabinosidase, Fucosidase, Galactosaminidase, Glucosaminidase, Glucosidase, Glucuronidase, Lactosidase, Maltosidase, Mannosidase, Xylosidase, arabinopyranoside, Fucopyranoside, Galactosaminide, Glucosaminide, Glucopyranoside, Glucuronic acid, Lactopyranoside, Maltopyranoside, Mannopyranoside, Xylopyranoside, 5-Bromo-4-chloro-3-indoxyl, 5-Bromo-6-chloro-3-indoxyl, 6-chloro-3-indoxyl, 5-Bromo-3-indoxyl, 5-Iodo-3-indoxyl, 3-indoxyl, 2-(6-Bromonaphthyl), 6-Fluoro-3-indoxyl 2-Nitrophenyl, 4-Nitrophenyl, 1-Naphthyl, Naphthyl AS-BI, 2-Nitrophenyl-N-acetyl, 4-Nitrophenyl-N-acetyl, 4-Methylumbelliferyl moieties, esterases, Carboxyl esterase, Cholesterol esterase, sulfatases, Aryl sufatase, phosphatases, Alkaline phosphatase, Carboxyl esterase, 6-chloro-3-indoxyl butyrate, Aryl sulfatase, 5-bromo-4-chloro-3-indoxyl sulfate, alkaline phosphatase 2-naphthyl, dehydrogenase, oxidase, hydroxylase, oxidoreductase, dehydrogenases, hydroxylases, a-NAD, nicotinamide adenine dinucleotide, nicotinamide adenine dinucleotide 3′-phosphate, nicotinamide adenine dinucleotide phosphate, triphosphopyridine, nicotinamide 1-N1-ethenoadenine dinucleotide phosphate, nicotinamide hypoxanthine dinucleotide, nicotinamide hypoxanthine dinucleotide phosphate, nicotinamide mononucleotide, nicotinamide N1-propylsulfonate, nicotinamide ribose monophosphate, Formaldehyde dehydrogenase, Fructose dehydrogenase, Glucose-6-phosphate dehydrogenase, Glucose dehydrogenase, Glutamate dehydrogenase, Glycerol dehydrogenase, Glycerol-3-phosphate dehydrogenase, hydroxybutyrate dehydrogenase, Hydroxybenzoate hydroxylase, Lactate dehydrogenase, Leucine dehydrogenase, Malate dehydrogenase, Mannitol dehydrogenase, oxidases, Acyl-CoA oxidase, alcohol oxidase, Ascorbate oxidase, Cholesterol oxidase, Choline oxidase, Glucose oxidase, Glycerophosphate oxidase, Xanthine oxidase, Uricase, peroxidase, pyrogallol, ABTS (2,2′-Azinobis(3-ethylbenzthiazoline)sulfonic acid), 3,3′,5,5′-Tetramethylbenzidine, ortho-Dianisidine, 3,3′-Diaminibenzidine, AEC (3-Amino-9-ethyl carbazole), 2-5, dimethyl-2,5-dihydroperoxyhexane, Bis{4-[N-(3′-sulfo-n-propyl)-N-n-ethyl]amino-2,6-dimethylphenyl}methane (Bis-MAPS), N-Ethyl-N-(2-hydroxy-3-sulfopropyl)-3-methoxyaniline (ADOS), N-Ethyl-N-(3-sulfopropyl)-3-methoxyaniline (ADPS), N-Ethyl-N-(2-hydroxy-3-sulfopropyl)aniline (ALOS), N-Ethyl-N-(3-sulfopropyl)-3,5-dimethylaniline (MAPS), N-Ethyl-N-(2-hydroxy-3-sulfopropyl)-3-methylaniline (TOOS), N-Ethyl-N-(3-sulfopropyl)-3-methylaniline (TOPS), N-(3-sulfopropyl)aniline (HALPS), N-Ethyl-N-(2-hydroxy-3-sulfopropyl)-3,5-dimethoxy-aniline (DAOS), N-Ethyl-N-(3-sulfopropyl)-3,5-dimethoxyaniline (DAPS), N-Ethyl-N-(3-sulfopropyl)aniline (ALPS), N-(2-hydroxy-3-sulfopropyl)-3,5-dimethoxyaniline (HDAOS), N-(3-sulfopropyl)-3,5-dimethoxyaniline (HDAPS), N-Ethyl-N-(2-hydroxy-3-sulfopropyl)-3,5-dimethylaniline (MAO), and N,N-Bis(4-sulfobutyl)-3,5-dimethylaniline (MADB), 3-Methyl-2-benzothiazolinonehydrazone, Dimerhylaniline, 4-aminoantipyrine, phenol, 2,4-Dichlorophenol, N,N-Diethyl-m-toluidine, p-Hydroxybenzene Sulfonate, N,N-Dimethylaniline, 3,5-Dichloro-2-Hydroxybenzenesulfonate, Sodium N-Ethyl-N-(3-Sulfopropyl)-m-Anisidine, N-Ethyl-N-(2-hydroxy-3-Sulfopropyl)-m-toluidine, NADPH oxidoreductase, NADPH, oxidoreductase, and NBT (nitro blue tetrazolium).

3. The method according to claim 1 wherein said buffer is selected from the group consisting of citrate, borate, phosphate, borax, sodium tetraborate decahydrate, sodium perchlorate, sodium chlorate, sodium carbonate, TRIS (Tris[hydroxymethyl]aminomethane), MES (2-[N-Morpholino]ethanesulfonic acid), BIS-TRIS (bis[2-Hydroxyethyl]iminotris[hydroxymethyl]methane;2-bis[2-Hydroxyethyl]amino-2-[hydroxymethyl-1,3-propanediol), ADA (N-[2-Acetamidol]-2-iminodiacetic acid; N-[Carbaoylmethyl]iminodiacetc acid), ACES (2-[(2-Amino-2-oxoethyl)amino]ethanesulfonic acid; N-[2-Acetamido]-2-aminoethanesulfonic acid), PIPES (PiperazineN-N′-bis[2-ethanesulfonic acid)]; 1,4-Piperzinedethanesulfoic acid), MOPSO (3-[N-Morpholinol]-2-hydroxypropanesulfonic acid), BIS-TRIS PROPANE (1,3-bis[tris(Hydroxymethyl)methylamino]propane), BES (N,N-bis[2-Hydroxyethyl]-2-aminoethaesulfonic acid; 2-bis(2-Hydroxyethyl)amino]ethanesulfonic acid), MOPS (3-[N-Morpholino]propanesulfonic acid), TES (N-tris[Hydroxymethyl]methyl-2-aminomethanesulfonic acid; 2[2-Hysroxy-1,1-bis(hydroxymethyl)-ethyl]amino)ethanesulfonic acid), HEPES (N-[2-Hydroxyethyl]piperazine-N′-[2-ethanesulfonic acid]), DIPSO (3-[N,N-bis(2-Hydroxyethyl)amino]-2-hydroxypropanesulfonic acid), TAPSO (3-[N-tris(Hydroxyethyl)methylamino]-2-hydroxypropanesulfonic acid), HEPPSO (N-[2-Hydroxythyl]piperazine-N′-[2Hydroxypropanesulfonic acid]), POPSO (Piperazine-N,N′-bis[2-hydroxypropanesulfonic acid]), EPPS (N-[2-Hydroxyethyl]piperazine-N′-[3-propanesulfonic acid), TEA (triethanolamine), TRICINE (N-tris[Hydroxymethyl]methyllycine; N-[2-Hydroxy-1-1-bis(hydroxymethyl)etyyl]glycine), BICINE (N,N-bis[2-Hydroxyethyl]glycine), TAPS (N-tris[Hydroxymethyl]methyl-3-aminopropanesulfonic acid; ([2-Hdroxy-1,1-bis(hydroxymethyl)ethyl]amino)-1-propanesulfonic acid), AMPSO (3-[(1,1-Dimethyl-2-hydroxyethyl)amino]-2-hydroxypropanesulfonic acid), CHES (2-[N-Cyclohexylamino]ethanesulfonic acid), CAPSO (3-[Cyclohexylamino]-2-hydroxy-1-propanesulfonic acid), AMP 2-Amino-2-ethyl-1-propanol, CAPS (3-[cyclohexylamino]-1-propanesulfonic acid), hydrochloric acid, phosphoric acid, lactic acid, sulfuric acid, nitric acid, chromic acid, boric acid, perchloric acid, potassium hydrogen tartrate, potassium hydrogen phthalate, calcium hydroxide, phosphate, bicarbonate, sodium hydroxide, potassium hydroxide, oxalate or succinate.

4. The method according to claim 1 wherein said nucleounit is not a nucleic acid ligand.

5. The method according to claim 1 wherein said target compound is not a protein.

6. The method according to claim 1 wherein the nucleounit indicator conjugate does not have to be bound to a solid support.

7. The method according to claim 1 wherein the analyte of interest is selected from the group consisting of cocaine, opiates, gamma-hydroxybutyric acid, cannabinoids, benzodiazepines, acetaminophen, amikacin, aminocaproic acid, amitriptyline, amobarbital, amphetamine, bromide, caffeine, carbamazepine, carbenicillin, chloral hydrate, chloramphenicol, chlordiazepoxide, chlorpromazine, cimetidine, clonazepam, clonidine, clorazepate, cocaine, cocaine metabolites, ethanol, methanol, or other forms of alcohol, codeine, cyclosporine, desipramine, dexamethsone, diazepam, digoxin, diphenylhydantoin, disopyramide, doxepin, ephedrine, ethchlorvynol, ethosuximide, fenoprofen, flecainide, flurazepam, gentamicin, glutethimide, hydromorphone, ibuprofen, imipramine, isoniazid, kanamycin, lidocaine, lithium, lorazepam, lysergic acid, meperidine, meprobamate, methadone, methamphetamine, methaqualone, methotrexate, methsuximide, methyldopa, methyprylon, morphine, n-acetylprocainamide, netilmicin, nortriptyline, oxazepam, oxycodone, paraldehyde, paraquat, pentazocine, pentobarbital, phenacetin, phencyclidine, phenobarbital, phensuximide, phenylbutazone, phenylpropanolamine, phenytoin, primidone, procainamide, propoxyphene, propranolol, protripyline, quinidine, salicylates, secobarbital, theophylline, thiocynate, thiopental, thioridazine, tobramycin, tolbutamide, valproic acid, vancomycin, cholesterol, triglycerides, glucose, adrenocortocotropic hormone, alanine, alanine aminotransferase, albumin, aldolase, aldosterone, amylase, amyloid-associated protein, androstenedione, angiotenesis, antidiuretic hormone, antithrombin, antitrypsin, apolipoprotein, ascorbic acid, bile acids, bilirubin, c-peptide, calcitonin, calcium, cancer antigen 125, carboxyhemoglobin, carotene, catecholamines, cholic acid, cholyglycine, chromium, chymotrypsin, complement components, coproporhyrin, corticobinding globulin, corticosterone, cortisol, c-peptide, c-reactive protein, creatine, creatinine, creatine kinase, cyclic AMP, cystine, cysteine, dehydroepiandrosterone, dehydroepiandrosterone sulfate, deoxycholic acid, 11-deoxycortocosterone, 11-deoxycortisol, dihydrotestosterone, estradiol, estriol, estrogen, estrone, fecal fat, fatty acids, ferritin, fetoprotein, fibrinogen, folate, follicle stimulating hormone, thyroxine, triiodothyronine, fructose, fructosamine, galactose, gastric acid, gastrin, glucagons, glucose-6-phosphate, glutamine, glutamyltransferase (GGT), glutathione, hemoglobin, glycerol, glycine, glycolic acid, gold, gonadptropins, growth hormone, haptoglobin, high-density lipoproteins, hemopexin, hemocystein, homocysteine, homogentisic acid, homovanillic acid, hydrogen sulfide, 17-hydroxycortocosteriods, 5-hydroxyindoleacetic acid, 17-hydroxyprogesterone, hydroxyproline, immunoglobins, insulin, iron, isocitrate dehydrogenase, isoleucine, 17-ketogenic steroids, ketone bodies, lactate, lactate dehydrogenase, lactose, LDL-cholesterol, lecithin, leucine, leukocyte, lipase, lipoproteins, lutropin, lysozyme, macroamylase, magnesium, melanin, metanephrine, methionine, metyrapone, microsomal antibodies, antibodies, molybdenum, mucoploysaccharide, myelin basic protein, myoglobin, methemoglobin, niacin, nickel, nitrite, nitrogen, nonprotein nitrogen, normetanephrine, blood, orosomucoid, oxalate, oxytocin, pancreatice polypeptide, pantothenic acid, parathyroid hormone, pentachloropehnol, pentoses, pepsinogen, phenols, phenolsulfonphthalein, phenylalanine, acid phosphatase, alkaline phosphatase, phosphofructokinase, phospholipids, placental lactogen, plasminogen, porphobilinogen, prealbumin, pregnanediol, chorionic gonadotropin, pregnanetriol, pregnenolone, progesterone, porinsulin, properdin, prostaglandins, prostate-specific antigen, portoporphyrin, pseudocholinesterase, pyruvic acid, renin, reverse triiodothyromine, rheumatoid factor, riboflavin, secretin, selenium, serotonin, somatomedin c, sucrose, testosterone, tetrahydrocortisol, tetrehydrodeoxycortisol, thallium, thyroglobin, thyroid antibodies, thyroid stimulating hormone, thyroxine binding globulin, thyroxine, transcortin, transferring, transketolase, transthyretin, thyrotropin-releasing hormone, triglycerides, triiodothryonine, tyrosine, urea, urea nitrogen, uric acid, uricase, urobilinogen, uroporphyrin, valine, vanillymandelic acid, vasoactive intestinal polypetide, human chorionic gonadotropin, mass creatinine kinase, vitamins, xylose, zinc, cyanide, formaldehyde, ethylene glycol, lead, mercury, xylene, human immunodeficiency virus (HIV), cytomegalovirus (CMV) IgG, cytomegalovirus (CMV) IgM, herpes simplex virus (types 1 and 2) IgG, rubella IgG, rubella, IgM, toxoplasma IgG, toxoplasma IgM, amebiasis, Epstein-barr early antigen, Epstein-barr EBNA IgG, Epstein-barr VCA IgG, Epstein-barr VCA IgM, helicobacter pylori-IgG, legionella IgG/IgM/IgA, mycoplasma IgG, mycoplasma IgM, varicella zoster virus (VZV), or autoimmune diseases such as the following but not limited to the following; antinuclear antibodies (ANA), antineutrophil cytoplasmic antibodies (ANCA), anti-cardiolipin, anti-dsDNA, anti-Jo-1, anti-Scl-70, anti-Sm (Smith antigen), anti-Sm/RNP, anti-SS-A/Ro, anti-SS-B/La, extractable nuclear antigen (ENA), myeloperoxidase IgG, proteinase-3 IgG, or Rheumatoid Factor.
